(19) United States
(12) Patent Application Publication
(10) Pub. No.: US 2011/0218103 A1
(43) Pub. Date: Sep. 8, 2011

(54) Fluoroalkyl-substituted 2-amidobenzimidazoles

(75) Inventors: Jens FRACKENPOHL, Frankfurt (DE); Ines HEINEMANN, Hofheim (DE); Thomas MÜLLER, Frankfurt (DE); Hans-Joachim ZEIß, Sulzbach (DE); Marco BUSCH, Burscheid-Osinghausen (DE); Lothar WILLMS, Hofheim (DE); Pascal VON KOSKULL-DÖRING, Frankfurt am Main (DE); Dirk SCHMUTZLER, Hattersheim (DE); Jan DITTGEN, Frankfurt (DE); Christopher Hugh ROSINGER, Hofheim (DE); Isolde HÄUSER-HAHN, Leverkusen (DE); Martin Jeffrey HILLS, Idstein (DE)

(73) Assignee: BAYER CROPSCIENCE AG, Monheim (DE)

(21) Appl. No.: 13/039,213

(22) Filed: Mar. 2, 2011

Related U.S. Application Data
(60) Provisional application No. 61/311,036, filed on Mar. 5, 2010.

(30) Foreign Application Priority Data
Mar. 4, 2010 (EP) 10155436.8

(51) Int. Cl. A01N 43/52; A01N 25/32; C07D 235/10; C07D 403/12; A01P 21/00; A01P 13/00; A01P 7/04; A01P 7/02; A01P 3/00; A01P 5/00

(52) U.S. Cl. 504/103; 504/276; 504/139; 548/309.4; 548/306.1

(57) ABSTRACT

Use of fluoroalkyl-substituted 2-amidobenzimidazoles of the formula (I), or salts thereof,

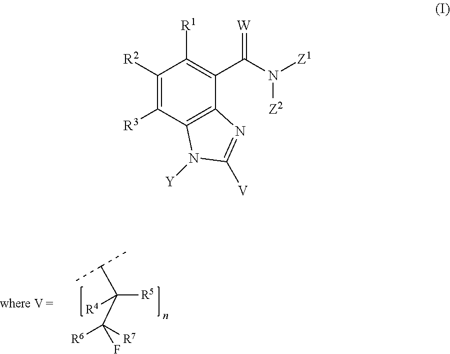

for enhancing stress tolerance in plants to abiotic stress, especially for strengthening plant growth and/or for increasing plant yield, and selected processes for preparing such fluoroalkyl-substituted 2-amidobenzimidazoles of the formula (I).

### CROSS REFERENCE TO RELATED APPLICATIONS

This application claims priority from EP 10155436.8 filed Mar. 4, 2010 and U.S. Provisional Application 61/311,036 filed Mar. 5, 2010, the contents of which are incorporated herein by reference in their entireties.

### BACKGROUND OF THE INVENTION

1. Field of the Invention

The invention relates to fluoroalkyl-substituted 2-am idobenzimidazoles and the analogs thereof, to processes for preparation thereof and to the use thereof for enhancing stress tolerance in plants to abiotic stress, especially for strengthening plant growth and/or for increasing plant yield.

2. Description of Related Art

It is known that particular phosphoric esters or carbamates, for example the compound O,S-dimethylthiolophosphoramide or the compound O-(2-isopropoxyphenyl)N-methylcarbamate, have insecticidal properties (cf., for example, DE1210835). The level of action or duration of action of these known compounds is, however, especially for particular insects or at low use concentrations, not entirely satisfactory in all fields of use.

It is additionally known that particular substituted benzimidazoles can be used as pesticides (cf. WO94/11349), but no compounds covered by the claims of the present application are disclosed explicitly.

It is also known that substituted amidobenzimidazoles can be used as active pharmaceutical ingredients (cf. WO2000029384) and for cosmetic uses (cf. WO2001082877). WO97/04771 likewise describes the pharmaceutical use of predominantly aryl-substituted benzimidazoles, while WO2000032579 describes heterocyclyl-substituted benzimidazoles.

It is known that plants react with specific or unspecific defense mechanisms to natural stress conditions, for example cold, heat, drought, injury, pathogenic attack (viruses, bacteria, fungi, insects) etc., but also to herbicides [Pflanzenbiochemie [Plant Biochemistry], pages 393-462, Spektrum Akademischer Verlag, Heidelberg, Berlin, Oxford, Hans W. Heldt, 1996.; Biochemistry and Molecular Biology of Plants, pages 1102-1203, American Society of Plant Physiologists, Rockville, Md., eds. Buchanan, Gruissem, Jones, 2000].

In plants, numerous proteins involved in defense reactions to abiotic stress (e.g. cold, heat, drought, salinity, flooding), and the genes that code for them, are known. Some of these form part of signal transduction chains (e.g. transcription factors, kinases, phosphatases) or cause a physiological response of the plant cell (e.g. ion transport, detoxification of reactive oxygen species). The signaling chain genes of the abiotic stress reaction include transcription factors of the DREB and CBF classes (Jaglo-Ottosen et al., 1998, Science 280: 104-106). Phosphatases of the ATPK and MP2C type are involved in the reaction to salt stress. In addition, in the event of salt stress, the biosynthesis of osmolytes such as proline or sucrose is frequently activated. For example, sucrose synthase and proline transporter are involved here (Hasegawa et al., 2000, Annu Rev Plant Physiol Plant Mol Biol 51: 463-499). The stress defense of the plants to cold and drought uses some of the same molecular mechanisms. The accumulation of “late embryogenesis abundant proteins” (LEA proteins) is known, one important class of which is that of the dehydrins (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which detoxify the reactive oxygen species (ROS) which arise in the event of oxidative stress (Kirch et al., 2005, Plant Mol Biol 57: 315-332).

Heat shock factors (HSFs) and heat shock proteins (HSPs) are activated in the event of heat stress and, as chaperones, play a similar role here to that of the dehydrins in the event of cold and drought stress (Yu et al., 2005, Mol Cells 19: 328-333).

A number of plant-endogeneous signaling substances involved in stress tolerance or pathogen defense are already known. Examples include salicylic acid, benzoic acid, jasmonic acid or ethylene [Biochemistry and Molecular Biology of Plants, pages 850-929, American Society of Plant Physiologists, Rockville, Md., eds. Buchanan, Gruissem, Jones, 2000]. Some of these substances or the stable synthetic derivatives thereof and derived structures are also effective in the case of external application to plants or seed dressing and activate defense reactions which result in an elevated stress or pathogen tolerance of the plant [Sembdner, and Parthier, 1993, Ann. Rev. Plant Physiol. Plant Mol. Biol. 44: 569-589].

It is additionally known that chemical substances can increase the tolerance of plants to abiotic stress. Such substances are applied by seed dressing, by leaf spraying or by soil treatment. For instance, an increase in abiotic stress tolerance of crop plants by treatment with elicitors of systemic acquired resistance (SAR) or abscisic acid derivatives is described (Schading and Wei, WO-200028055, Abrams and Gusta, U.S. Pat. No. 5,201,931, Churchill et al., 1998, Plant Growth Regul 25: 35-45) or azibenzolar-S-methyl. In the case of use of fungicides, especially from the group of the strobilurins or of the succinate dehydrogenase inhibitors, similar effects are also observed, and are frequently also accompanied by an enhanced yield (Draber et al., DE-3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In addition, effects of growth regulators on the stress tolerance of crop plants have been described (Morrison and Andrews, 1992, J Plant Growth Regul 11: 113-117, RD-259027). In the event of osmotic stress, a protective effect resulting from application of osmolytes, for example glycine betaine or the biochemical precursors thereof, for example choline derivatives, has been observed (Chen et al., 2000, Plant Cell Environ 23: 609-618, Bergmann et al., DE-4103253). The effect of antioxidants, for example naphthols and xanthines, to increase abiotic stress tolerance in plants has also already been described (Bergmann et al., DD-277832, Bergmann et al., DD-277835). The molecular causes of the antistress action of these substances are, however, largely unknown.

It is additionally known that the tolerance of plants to abiotic stress can be increased by a modification of the activity of endogeneous poly-ADP-ribose polymerases (PARP) or poly-(ADP-ribose) glycohydrolases (PARG) (de Block et al., The Plant Journal, 2005, 41, 95; Levine et al., FEBS Lett. 1998, 440, 1; WO0004173; WO04090140).

It is thus known that plants possess several endogenous reaction mechanisms which can cause effective defense against a wide variety of harmful organisms and/or natural abiotic stress.

Since, however, the ecologic and economic demands on modern crop treatment compositions are increasing constantly, for example with respect to toxicity, selectivity, application rate, formation of residues and favourable manufacture, there is a constant need to develop novel crop treatment compositions which have advantages over those known, at least in some areas.

### SUMMARY OF THE INVENTION

It was therefore an object of the present invention to provide further compounds which increase tolerance to abiotic stress in plants.

The present invention accordingly provides for the use of fluoroalkyl-substituted 2-amidobenzimidazoles of the formula (I), or salts thereof,

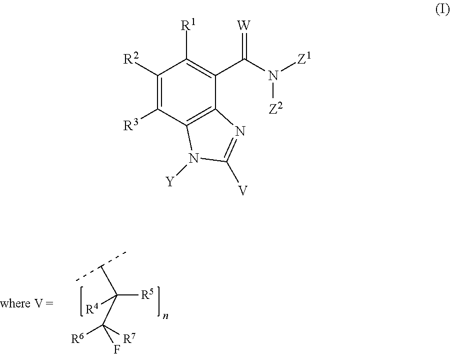

for increasing tolerance to abiotic stress in plants, where
R¹, R², R³ are each independently H, halogen, branched or unbranched alkyl, cycloalkyl, alkenyl, alkynyl, aryl, arylalkyl, heteroaryl, alkoxyalkyl, alkylthio, fluoroalkylthio, haloalkyl, alkoxy, haloalkoxy;
R⁴ is H, unbranched alkyl, halogen, haloalkyl, branched alkyl, unbranched alkenyl, branched alkenyl, cycloalkyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁵ is H, unbranched alkyl, halogen, haloalkyl, branched alkyl, cycloalkyl, cycloalkenyl, alkoxy, haloalkoxy;
R⁶ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁷ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
n is 0, 1, 2, 3, 4, 5, 6;
W is oxygen, sulfur;
Y is H, unbranched or branched alkyl, cycloalkyl, cycloalkylalkyl, cycloalkenyl, cyanoalkyl, unbranched or branched alkenylalkyl, branched or unbranched haloalkyl, alkynylalkyl, arylalkyl, heteroarylalkyl, alkylcarbonyl, cycloalkylcarbonyl, arylcarbonyl, arylalkylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, alkoxyalkyl;
Z¹ is H, unbranched or branched alkyl, cycloalkyl, halogen, unbranched or branched alkylalkenyl, branched or unbranched haloalkyl, alkynyl, alkenyl, cyanoalkyl, arylalkyl, heteroarylalkyl, alkylcarbonyl, alkoxycarbonyl, alkylsulfonyl, arylsulfonyl, cycloalkylsulfonyl, alkylsulfinyl, arylsulfinyl, cycloalkylsulfinyl, alkoxycarbonylalkyl;
   and
Z² is H, unbranched or branched alkyl, cycloalkyl, unbranched or branched alkylalkenyl, branched or unbranched haloalkyl, alkynyl, alkenyl, cyanoalkyl, arylalkyl, heteroarylalkyl, alkylcarbonyl, alkoxycarbonyl;
   or
Z¹ and Z² together are part of an optionally substituted sulfilimine or amidine group or form an iminophosphorane.

### DETAILED DESCRIPTION OF A PREFERRED EMBODIMENT

The compounds of the formula (I) can form salts by addition of a suitable inorganic or organic acid, for example mineral acids, for example HCl, HBr, H₂SO₄, H₃PO₄or HNO₃, or organic acids, for example carboxylic acids such as formic acid, acetic acid, propionic acid, oxalic acid, lactic acid or salicylic acid, or sulfonic acids, for example p-toluenesulfonic acid, onto a basic group, for example amino, alkylamino, dialkylamino, piperidino, morpholino or pyridino. These salts then contain the conjugate base of the acid as the anion.

Suitable substituents present in deprotonated form, for example sulfonic acids or carboxylic acids, can form internal salts with groups which are themselves protonable, such as amino groups.

The inventive compounds of the formula (I) and salts thereof and/or those used in accordance with the invention are also referred to hereinafter as “compounds of the formula (I)” for short.

Preference is given to the inventive use of compounds of the formula (I) or salts thereof, in which
R¹, R², R³ are each independently H, fluorine, chlorine, bromine, iodine, branched or unbranched (C₁-C₆)-alkyl, (C₃-C₇)-cycloalkyl, unbranched (C₂-C₆)-alkenyl, branched (C₃-C₆)-alkenyl, (C₂-C₆)-alkynyl, aryl, heteroaryl, aryl-(C₁-C₆)-alkyl, (C₁-C₆)-alkoxy-(C₁-C₆)-alkyl, (C₁-C₆)-alkylthio, (C₁-C₆)-fluoroalkylthio, (C₁-C₆)-haloalkyl, (C₁-C₆)-alkoxy, (C₁-C₆)-haloalkoxy;
R⁴ is H, unbranched (C₁-C₆)-alkyl, fluorine, chlorine, bromine, iodine, (C₁-C₆)-haloalkyl, branched (C₃-C₆)-alkyl, unbranched (C₂-C₆)-alkenyl, branched (C₃-C₆)-alkenyl, (C₃-C₇)-cycloalkyl, (C₁-C₈)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino(C₁-C₆)-alkoxy;
R⁵ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl;
R⁶ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl, (C₁-C₈)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino(C₁-C₆)-alkoxy;
R⁷ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl
n is 0, 1, 2, 3, 4, 5;
W is oxygen, sulfur;
Y is H, unbranched or branched (C₁-C₈)-alkyl, (C₃-C₇)-cycloalkyl, (C₃-C₇)-cycloalkyl-(C₁-C₆)-alkyl, (C₁-C₈)-cyanoalkyl, (C₃-C₇)-cycloalkenyl, unbranched or branched (C₂-C₆)-alkenyl-(C₁-C₆)-alkyl, (C₁-C₆)-haloalkyl, (C₂-C₆)-alkynyl-(C₁-C₆)-alkyl, aryl-(C₁-C₆)-alkyl, heteroaryl-(C₁-C₆)-alkyl, (C₁-C₆)-alkylcarbonyl, (C₃-C₇)-cycloalkylcarbonyl, arylcarbonyl, aryl-(C₁-C₆)-alkylcarbonyl, (C₁-C₆)-alkoxycarbonyl, (C₁-C₆)-alkylsulfonyl, (C₃-C₇)-cycloalkylsulfonyl, arylsulfonyl, heteroarylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, (C₁-C₆)-alkoxy-(C₁-C₆)-alkyl;
Z¹ is H, (C₁-C₈)-alkyl, (C₃-C₇)-cycloalkyl, chlorine, bromine, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl, (C₁-C₆)-alkylsulfonyl, arylsulfonyl, (C₃-C₇)-cycloalkylsulfonyl, (C₁-C₆)-alkylsulfinyl, arylsulfinyl, (C₃-C₇)-cycloalkylsulfinyl, (C₁-C₆)-alkoxycarbonyl-(C₁-C₆)-alkyl;
   and
Z² is H, (C₁-C₈)-alkyl, (C₃-C₇)-cycloalkyl, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl;
   or

Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

Particular preference is given to the inventive use of compounds of the formula (I) or salts thereof, in which
R¹, R², R³ are each independently H, fluorine, chlorine, bromine, iodine, branched or un branched (C₁-C₄)-alkyl, (C₃-C₆)-cycloalkyl, (C₂-C₄)-alkenyl, (C₂-C₄)-alkynyl, aryl, heteroaryl, aryl-(C₁-C₄)-alkyl, (C₁-C₄)-alkoxy-(C₁-C₄)-alkyl, (C₁-C₄)-alkylthio, (C₁-C₄)-fluoroalkylthio, (C₁-C₄)-haloalkyl, (C₁-C₄)-alkoxy, (C₁-C₄)-haloalkoxy;
R⁴ is H, unbranched (C₁-C₄)-alkyl, fluorine, chlorine, bromine, (C₁-C₄)-haloalkyl, branched (C₃-C₆)-alkyl, unbranched (C₂-C₄)-alkenyl, branched (C₃-C₆)-alkenyl, (C₃-C₆)-cycloalkyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkyloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino(C₁-C₄)-alkoxy;
R⁵ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl;

R⁶ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkyloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino(C₁-C₄)-alkoxy;
R⁷ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl
n is 0, 1, 2, 3, 4;
W is oxygen, sulfur;
Y is H, unbranched or branched (C₁-C₆)-alkyl, (C₃-C₆)-cycloalkyl, (C₃-C₆)-cycloalkyl-(C₁-C₄)-alkyl, (C₃-C₆)-cycloalkenyl, (C₁-C₄)-cyanoalkyl, unbranched or branched (C₂-C₄)-alkenyl-(C₁-C₄)-alkyl, (C₁-C₄)-haloalkyl, (C₂-C₄)-alkynyl-(C₁-C₄)-alkyl, aryl-(C₁-C₄)-alkyl, heteroaryl-(C₁-C₄)-alkyl, (C₁-C₅)-alkylcarbonyl, (C₃-C₆)-cycloalkylcarbonyl, arylcarbonyl, aryl-(C₁-C₄)-alkylcarbonyl, (C₁-C₄)-alkoxycarbonyl, (C₁-C₄)-alkylsulfonyl, (C₃-C₆)-cycloalkylsulfonyl, arylsulfonyl, heteroarylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, (C₁-C₄)-alkoxy-(C₁-C₄)-alkyl;

Z¹ is H, (C₁-C₆)-alkyl, (C₃-C₆)-cycloalkyl, chlorine, bromine, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl, (C₁-C₄)-alkylsulfonyl, arylsulfonyl, (C₃-C₆)-cycloalkylsulfonyl, (C₁-C₄)-alkylsulfinyl, arylsulfinyl, (C₃-C₆)-cycloalkylsulfinyl, (C₁-C₄)-alkoxycarbonyl-(C₁-C₄)-alkyl;

and
Z² is H, (C₁-C₆)-alkyl, (C₃-C₆)-cycloalkyl, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl;
   or
Z¹ and Z² together form an N-(bis(C₁-C₅)-alkyl)sulfanylidene, N-(aryl-(C₁-C₅)-alkyl)sulfanylidene, N-(bis(C₃-C₆)-cycloalkyl)sulfanylidene, N-((C₁-C₅)-alkyl-(C₃-C₆)-cycloalkyl)sulfanylidene group or an N,N-di-(C₁-C₄)-alkylformylidene group.

Very particular preference is given to the inventive use of compounds of the formula (I) or salts thereof, in which
R₁, R₂, R₃ are each independently H, F, Cl, Br, I, CH₃, CF₃, OCH₃, OCF₃
W is oxygen, sulfur;
n is 0, 1, 2, 3, 4
Y is H, methyl, ethyl, n-propyl, n-butyl, isobutyl, isopropyl, n-pentyl, n-hexyl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl;
V represents fluoroalkyls having 1 to 4 carbon atoms and 1 to 9, preferably 1 to 6, identical or different halogen atoms with at least one fluorine atom, i.e. partly fluorinated alkyl, perfluoroalkyl, partly fluorinated haloalkyl, where any other halogen atoms present are selected from the group of fluorine, chlorine and bromine, preferably trifluoromethyl, pentafluoroethyl, heptafluoropropyl, nonafluorobutyl, chlorodifluoromethyl, bromodifluoromethyl, dichlorofluoromethyl, bromofluoromethyl, 1-fluoroethyl, 2-fluoroethyl, fluoromethyl, difluoromethyl, 2,2-difluoroethyl, 2,2,2-trifluoroethyl, 2,2-dichloro-2-fluoroethyl, 2-chloro-2,2-difluoroethyl, difluoro-tert-butyl, 1-fluorocyclopropyl, 2-fluorocyclopropyl, 2-fluoro-2-chlorocyclopropyl, 2-bromo-1,1,2-trifluoroethyl, 1,1,2,2-tetrafluoroethyl, 1,2,2,2-tetrafluoroethyl, 2-chloro-1,1,2-trifluoroethyl, 2-chloro-1,1,2,2-tetrafluoroethyl, 1,2,2,3,3,3-hexafluoropropyl, 1-methyl-2,2,2-trifluoroethyl, 1-chloro-2,2,2-trifluoroethyl, 1,2,2,3,3,4,4,4-octafluorobutyl, 1-fluoro-1-methylethyl, n-propoxydifluoromethyl, methoxydifluoromethyl, ethoxydifluoromethyl, n-butoxydifluoromethyl, methoxyethoxydifluoromethyl, n-pentoxydifluoromethyl, 2-methylbutoxydifluoromethyl, 4-methylpentoxydifluoromethyl, n-hexyloxydifluoromethyl, isohexyloxydifluoromethyl, allyloxypropoxydifluoromethyl, methoxypropoxydifluoromethyl, cyclopropylmethoxydifluoromethyl, cyclobutylmethoxydifluoromethyl, but-3-yn-1-yloxydifluoromethyl, pent-4-yn-1-yloxydifluoromethyl, hex-3-yn-1-yloxydifluoromethyl, but-3-en-1-yloxydifluoromethyl, 2,2,2-trifluoroethoxydifluoromethyl, 3,3,3-trifluoropropoxydifluoromethyl, 4,4,4-trifluorobutoxydifluoromethyl, 4-dimethylaminobutoxydifluoromethyl, 2-(1-methylpyrrolidin-2-yl)ethoxydifluoromethyl;
Z¹ is H, chlorine, methyl, ethyl, n-propyl, n-butyl, isobutyl, isopropyl, n-pentyl, n-hexyl, cyclopropyl, cyclobutyl, cyclopentyl, cyclohexyl, prop-1-yn-3-yl, but-2-yn-3-yl, cyanomethyl, prop-1-en-3-yl, but-1-en-4-yl, methylsulfonyl, ethylsulfonyl, cyclopropylsulfonyl, isopropylsulfonyl, n-propylsulfonyl, phenylsulfonyl, p-chlorophenylsulfonyl, m-chlorophenylsulfonyl, m,p-dichlorophenylsulfonyl, p-iodophenylsulfonyl, p-trifluoromethoxyphenylsulfonyl, p-methylphenylsulfonyl; methoxycarbonylmethyl, 1-methoxycarbonylethyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
   and
Z² is H, methyl, ethyl, n-propyl, n-butyl, n-pentyl, n-hexyl, cyclopropyl, cyclobutyl, prop-1-yn-3-yl, but-2-yn-3-yl, cyanomethyl, prop-1-en-3-yl, but-1-en-4-yl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
   or
Z¹ and Z² together are N-(di-n-butylsulfanylidene), N-(diisopropylsulfanylidene), N-(di-n-propylsulfanylidene), N-(di-n-pentylsulfanylidene), N-(diisobutylsulfanylidene), N-(cyclobutylisopropylsulfanylidene), N-(n-propylisopropylsulfanylidene), N-(cyclopropylisopropylsulfanylidene), N-(isobutylisopropylsulfanylidene), N,N-dimethylformylidene.

The aforementioned fluoroalkyl-substituted 2-amidobenzimidazoles of the formula (I) are essentially likewise as yet unknown in the prior art.

Thus, a further part of the invention is that of fluoroalkyl-substituted 2-amidobenzimidazoles of the formula (I), or salts thereof,

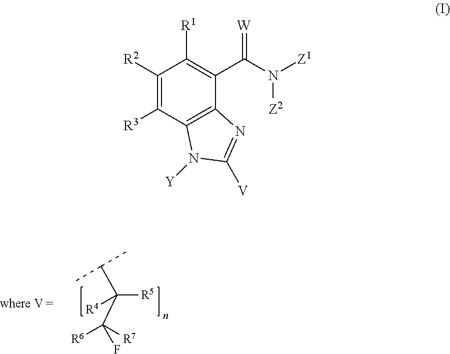

in which
R¹, R², R³ are each independently H, halogen, branched or unbranched alkyl, cycloalkyl, alkenyl, alkynyl, aryl, arylalkyl, heteroaryl, alkoxyalkyl, alkylthio, fluoroalkylthio, haloalkyl, alkoxy, haloalkoxy;
R⁴ is H, unbranched alkyl, halogen, haloalkyl, branched alkyl, unbranched alkenyl, branched alkenyl, cycloalkyl, alkoxy, haloalkoxy, cycloalkylalkoxy, alkynylalkoxy, alkenylalkoxy, alkenyloxyalkoxy, alkyloxyalkoxy, alkylaminoalkoxy,
R⁵ is H, unbranched alkyl, halogen, haloalkyl, branched alkyl, cycloalkyl, cycloalkenyl, alkoxy, haloalkoxy;
R⁶ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁷ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
n is 0, 1, 2, 3, 4, 5, 6;
W is oxygen, sulfur;
Y is H, unbranched or branched alkyl, cycloalkyl, cycloalkylalkyl, cycloalkenyl, cyanoalkyl, unbranched or branched alkenylalkyl, branched or unbranched haloalkyl, alkynylalkyl, arylalkyl, heteroarylalkyl, alkylcarbonyl, cycloalkylcarbonyl, arylcarbonyl, arylalkylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, alkoxyalkyl, cyanoalkyl;
Z¹ is H, unbranched or branched alkyl, cycloalkyl, halogen, unbranched or branched alkylalkenyl, branched or unbranched haloalkyl, alkynyl, alkenyl, cyanoalkyl, arylalkyl, heteroarylalkyl, alkylcarbonyl, alkoxycarbonyl, alkylsulfonyl, arylsulfonyl, cycloalkylsulfonyl, alkylsulfinyl, arylsulfinyl, cycloalkylsulfinyl, alkoxycarbonylalkyl;
   and
Z² is H, unbranched or branched alkyl, cycloalkyl, branched or unbranched haloalkyl, alkynyl, alkenyl, cyanoalkyl, arylalkyl, heteroarylalkyl, unbranched or branched alkylalkenyl, alkylcarbonyl, alkoxycarbonyl;
   or
Z¹ and Z² together are part of an optionally substituted sulfilimine or amidine group or form an iminophosphorane,

excluding the compound of the formula (I) in which R¹, R² and R³ are each H; Z¹ and Z² are each H; W is O; Y is H; and V is CF₃.

Preference is given to compounds of the formula (I) or salts thereof, in which
R¹, R², R³ are each independently H, fluorine, chlorine, bromine, iodine, branched or unbranched (C₁-C₆)-alkyl, (C₃-C₇)-cycloalkyl, unbranched (C₂-C₆)-alkenyl, branched (C₃-C₆)-alkenyl, (C₂-C₆)-alkynyl, aryl, heteroaryl, aryl-(C₁-C₆)-alkyl, (C₁-C₆)-alkoxy-(C₁-C₆)-alkyl, (C₁-C₆)-alkylthio, (C₁-C₆)-fluoroalkylthio, (C₁-C₆)-haloalkyl, (C₁-C₆)-alkoxy, (C₁-C₆)-haloalkoxy;
R⁴ is H, unbranched (C₁-C₆)-alkyl, fluorine, chlorine, bromine, iodine, (C₁-C₆)-haloalkyl, branched (C₃-C₆)-alkyl, unbranched (C₂-C₆)-alkenyl, branched (C₃-C₆)-alkenyl, (C₃-C₇)-cycloalkyl, (C₁-C₈)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino(C₁-C₆)-alkoxy;
R⁵ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl;
R⁶ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl, (C₁-C₈)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino(C₁-C₆)-alkoxy;
R⁷ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl
n is 0, 1, 2, 3, 4, 5;
W is oxygen, sulfur;
Y is H, unbranched or branched (C₁-C₈)-alkyl, (C₃-C₇)-cycloalkyl, (C₃-C₇)-cycloalkyl-(C₁-C₆)-alkyl, (C₁-C₈)-cyanoalkyl, (C₃-C₇)-cycloalkenyl, unbranched or branched (C₂-C₆)-alkenyl-(C₁-C₆)-alkyl, (C₁-C₆)-haloalkyl, (C₂-C₆)-alkynyl-(C₁-C₆)-alkyl, aryl-(C₁-C₆)-alkyl, heteroaryl-(C₁-C₆)-alkyl, (C₁-C₆)-alkylcarbonyl, (C₃-C₇)-cycloalkylcarbonyl, arylcarbonyl, aryl-(C₁-C₆)-alkylcarbonyl, (C₁-C₆)-alkoxycarbonyl, (C₁-C₆)-alkylsulfonyl, (C₃-C₇)-cycloalkylsulfonyl, arylsulfonyl, heteroarylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, (C₁-C₆)-alkoxy-(C₁-C₆)-alkyl;
Z¹ is H, (C₁-C₈)-alkyl, (C₃-C₇)-cycloalkyl, chlorine, bromine, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl, (C₁-C₆)-alkylsulfonyl, arylsulfonyl, (C₃-C₇)-cycloalkylsulfonyl, (C₁-C₆)-alkylsulfinyl, arylsulfinyl, (C₃-C₇)-cycloalkylsulfinyl, (C₁-C₆)-alkoxycarbonyl-(C₁-C₆)-alkyl;
   and
Z² is H, (C₁-C₈)-alkyl, (C₃-C₇)-cycloalkyl, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl;
   or

Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

excluding the compound of the formula (I) in which

R¹, R² and R³ are each H; Z¹ and Z² are each H; W is O; Y is H; and V is CF₃.

Particular preference is given to compounds of the formula (I) or salts thereof, in which
R¹, R², R³ are each independently H, fluorine, chlorine, bromine, iodine, branched or unbranched (C₁-C₄)-alkyl, (C₃-C₆)-cycloalkyl, (C₂-C₄)-alkenyl, (C₂-C₄)-alkynyl, aryl, heteroaryl, aryl-(C₁-C₄)-alkyl, (C₁-C₄)-alkoxy-(C₁-C₄)-alkyl, (C₁-C₄)-alkylthio, (C₁-C₄)-fluoroalkylthio, (C₁-C₄)-haloalkyl, (C₁-C₄)-alkoxy, (C₁-C₄)-haloalkoxy;
R⁴ is H, unbranched (C₁-C₄)-alkyl, fluorine, chlorine, bromine, (C₁-C₄)-haloalkyl, branched (C₃-C₆)-alkyl, unbranched (C₂-C₄)-alkenyl, branched (C₃-C₆)-alkenyl, (C₃-C₆)-cycloalkyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkyloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino(C₁-C₄)-alkoxy;
R⁵ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl;
R⁶ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkyloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino(C₁-C₄)-alkoxy;
R⁷ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl
n is 0, 1, 2, 3, 4;
W is oxygen, sulfur;
Y is H, unbranched or branched (C₁-C₆)-alkyl, (C₃-C₆)-cycloalkyl, (C₃-C₆)-cycloalkyl-(C₁-C₄)-alkyl, (C₃-C₆)-cycloalkenyl, (C₁-C₄)-cyanoalkyl, unbranched or branched (C₂-C₄)-alkenyl-(C₁-C₄)-alkyl, (C₁-C₄)-haloalkyl, (C₂-C₄)-alkynyl-(C₁-C₄)-alkyl, aryl-(C₁-C₄)-alkyl, heteroaryl-(C₁-C₄)-alkyl, (C₁-C₅)-alkylcarbonyl, (C₃-C₆)-cycloalkylcarbonyl, arylcarbonyl, aryl-(C₁-C₄)-alkylcarbonyl, (C₁-C₄)-alkoxycarbonyl, (C₁-C₄)-alkylsulfonyl, (C₃-C₆)-cycloalkylsulfonyl, arylsulfonyl, heteroarylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, (C₁-C₄)-alkoxy-(C₁-C₄)-alkyl;
Z¹ is H, (C₁-C₆)-alkyl, (C₃-C₆)-cycloalkyl, chlorine, bromine, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl, (C₁-C₄)-alkylsulfonyl, arylsulfonyl, (C₃-C₆)-cycloalkylsulfonyl, (C₁-C₄)-alkylsulfinyl, arylsulfinyl, (C₃-C₆)-cycloalkylsulfinyl, (C₁-C₄)-alkoxycarbonyl-(C₁-C₄)-alkyl;

and
Z² is H, (C₁-C₆)-alkyl, (C₃-C₆)-cycloalkyl, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl;
   or
Z¹ and Z² together form an N-(bis(C₁-C₅)-alkyl)sulfanylidene, N-(aryl-(C₁-C₅)-alkyl)sulfanylidene, N-(bis(C₃-C₆)-cycloalkyl)sulfanylidene, N—((C₁-C₅)-alkyl-(C₃-C₆)-cycloalkyl)sulfanylidene group or an N,N-di-(C₁-C₄)-alkylformylidene group.

excluding the compound of the formula (I) in which

R¹, R² and R³ are each H; Z¹ and Z² are each H; W is O; Y is H; and V is CF₃.

Very particular preference is given to compounds of the formula (I) or salts thereof, in which

R₁, R₂, R₃ are each independently H, F, Cl, Br, I, CH₃, CF₃, OCH₃, OCF₃
W is oxygen, sulfur;
n is 0, 1, 2, 3, 4
Y is H, methyl, ethyl, n-propyl, n-butyl, isobutyl, isopropyl, n-pentyl, n-hexyl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl;
V represents fluoroalkyls having 1 to 4 carbon atoms and 1 to 9, preferably 1 to 6, identical or different halogen atoms with at least one fluorine atom, i.e. partly fluorinated alkyl, perfluoroalkyl, partly fluorinated haloalkyl, where any other halogen atoms present are selected from the group of fluorine, chlorine and bromine, preferably trifluoromethyl, pentafluoroethyl, heptafluoropropyl, nonafluorobutyl, chlorodifluoromethyl, bromodifluoromethyl, dichlorofluoromethyl, bromofluoromethyl, 1-fluoroethyl, 2-fluoroethyl, fluoromethyl, difluoromethyl, 2,2-difluoroethyl, 2,2,2-trifluoroethyl, 2,2-dichloro-2-fluoroethyl, 2-chloro-2,2-difluoroethyl, difluoro-tert-butyl, 1-fluorocyclopropyl, 2-fluorocyclopropyl, 2-fluoro-2-chlorocyclopropyl, 2-bromo-1,1,2-trifluoroethyl, 1,1,2,2-tetrafluoroethyl, 1,2,2,2-tetrafluoroethyl, 2-chloro-1,1,2-trifluoroethyl, 2-chloro-1,1,2,2-tetrafluoroethyl, 1,2,2,3,3,3-hexafluoropropyl, 1-methyl-2,2,2-trifluoroethyl, 1-chloro-2,2,2-trifluoroethyl, 1,2,2,3,3,4,4,4-octafluorobutyl, 1-fluoro-1-methylethyl, n-propoxydifluoromethyl, methoxydifluoromethyl, ethoxydifluoromethyl, n-butoxydifluoromethyl, methoxyethoxydifluoromethyl, n-pentoxydifluoromethyl, 2-methylbutoxydifluoromethyl, 4-methylpentoxydifluoromethyl, n-hexyloxydifluoromethyl, isohexyloxydifluoromethyl, allyloxypropoxydifluoromethyl, methoxypropoxydifluoromethyl, cyclopropylmethoxydifluoromethyl, cyclobutylmethoxydifluoromethyl, but-3-yn-1-yloxydifluoromethyl, pent-4-yn-1-yloxydifluoromethyl, hex-3-yn-1-yloxydifluoromethyl, but-3-en-1-yloxydifluoromethyl, 2,2,2-trifluoroethoxydifluoromethyl, 3,3,3-trifluoropropoxydifluoromethyl, 4,4,4-trifluorobutoxydifluoromethyl, 4-dimethylaminobutoxydifluoromethyl, 2-(1-methylpyrrolidin-2-yl)ethoxydifluoromethyl;
Z¹ is H, chlorine, methyl, ethyl, n-propyl, n-butyl, isobutyl, isopropyl, n-pentyl, n-hexyl, cyclopropyl, cyclobutyl, cyclopentyl, cyclohexyl, prop-1-yn-3-yl, but-2-yn-3-yl, cyanomethyl, prop-1-en-3-yl, but-1-en-4-yl, methylsulfonyl, ethylsulfonyl, cyclopropylsulfonyl, isopropylsulfonyl, n-propylsulfonyl, phenylsulfonyl, p-chlorophenylsulfonyl, m-chlorophenylsulfonyl, m,p-dichlorophenylsulfonyl, p-iodophenylsulfonyl, p-trifluoromethoxyphenylsulfonyl, p-methylphenylsulfonyl; methoxycarbonylmethyl, 1-methoxycarbonylethyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
   and
Z² is H, methyl, ethyl, n-propyl, n-butyl, n-pentyl, n-hexyl, cyclopropyl, cyclobutyl, prop-1-yn-3-yl, but-2-yn-3-yl, cyanomethyl, prop-1-en-3-yl, but-1-en-4-yl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
   or
Z¹ and Z² together are N-(di-n-butylsulfanylidene), N-(diisopropylsulfanylidene), N-(di-n-propylsulfanylidene), N-(di-n-pentylsulfanylidene), N-(diisobutylsulfanylidene), N-(cyclobutylisopropylsulfanylidene), N-(n-propylisopropylsulfanylidene), N-(cyclopropylisopropylsulfanylidene), N-(isobutylisopropylsulfanylidene), N,N-dimethylformylidene.

excluding the compound of the formula (I) in which

R¹, R² and R³ are each H; Z¹ and Z² are each H; W is O; Y is H; and V is CF₃.

Thus, the invention further provides a spray solution for treatment of plants, comprising an amount, effective for enhancement of the resistance of plants to abiotic stress factors, of one or more of the aforementioned fluoroalkyl-substituted 2-amidobenzimidazoles, excluding the compound of the formula (I) in which R¹, R² and R³ are each H; Z¹ and Z² are each H; W is O; Y is H; and V is CF₃.

With regard to the inventive compounds, the terms used above and below will be elucidated. These are familiar to the person skilled in the art and have especially the definitions elucidated hereinafter:

The term “halogen” means, for example, fluorine, chlorine, bromine or iodine. When the term is used for a radical, “halogen” means, for example, a fluorine, chlorine, bromine or iodine atom.

Alkyl means a straight-chain or branched open-chain, saturated hydrocarbyl radical which has optionally been mono- or polysubstituted. Preferred substituents are halogen atoms, alkoxy groups, haloalkoxy groups, cyano groups, alkylthio groups, haloalkylthio groups or nitro groups, particular preference being given to fluorine, chlorine, bromine or iodine.

Fluoroalkyl means a straight-chain or branched open-chain, saturated and fluorine-substituted hydrocarbyl radical, where at least one fluorine atom is at one of the possible positions.

Perfluoroalkyl means a straight-chain or branched open-chain, saturated and fully fluorine-substituted hydrocarbyl radical, for example CF₃, CF₂CF₃, CF₂CF₂CF₃.

Partly fluorinated alkyl means a straight-chain or branched, saturated hydrocarbyl radical which has been mono- or polysubstituted by fluorine, where the corresponding fluorine atoms may be present as substituents on one or more different carbon atoms of the straight-chain or branched hydrocarbyl chain, for example CHFCH₃, CH₂CH₂F, CH₂CH₂CF₃, CHF₂, CH₂F, CHFCF₂CF₃.

Partly fluorinated haloalkyl means a straight-chain or branched, saturated hydrocarbyl radical which has been substituted by different halogen atoms with at least one fluorine atom, where any other halogen atoms present are selected from the group of fluorine, chlorine, bromine and iodine. The corresponding halogen atoms may be present as substituents on one or more different carbon atoms of the straight-chain or branched hydrocarbyl chain. Partly fluorinated haloalkyl also includes full substitution of the straight or branched chain by halogen including at least one fluorine atom.

Haloalkyl, -alkenyl and -alkynyl mean alkyl, alkenyl or alkynyl partly or fully substituted by identical or different halogen atoms, for example monohaloalkyl, for example CH₂CH₂Cl, CH₂CH₂Br, CHClCH₃, CH₂Cl, CH₂F; perhaloalkyl, for example CCl₃, CClF₂, CFCl₂, CF₂CClF₂, CF₂CClFCF₃; polyhaloalkyl, for example CH₂CHFCl, CF₂CClFH, CF₂CBrFH, CH₂CF₃; the term “perhaloalkyl” also includes the term “perfluoroalkyl”, and the term “polyhaloalkyl” also includes the terms “partly fluorinated alkyl” and “partly fluorinated haloalkyl”.

Haloalkoxy is, for example, OCF₃, OCHF₂, OCH₂F, OCF₂CF₃, OCH₂CF₃ and OCH₂CH₂Cl; the situation is corresponding for haloalkenyl and other halogen-substituted radicals.

The expression “(C₁-C₄)-alkyl” is abbreviated notation for alkyl having one to four carbon atoms according to the range stated for carbon atoms, i.e. includes the methyl, ethyl, 1-propyl, 2-propyl, 1-butyl, 2-butyl, 2-methylpropyl or tert-butyl radicals. General alkyl radicals having a wider specified range of carbon atoms, for example “(C₁-C₆)-alkyl”, correspondingly also include straight-chain or branched alkyl radicals with a greater number of carbon atoms, i.e. according to the example also the alkyl radicals with 5 and 6 carbon atoms.

Unless stated specifically, in the case of the hydrocarbyl radicals such as alkyl, alkenyl and alkynyl radicals, including in combined radicals, the lower carbon skeletons, for example having 1 to 6 carbon atoms, or in the case of unsaturated groups having 2 to 6 carbon atoms, are preferred. Alkyl radicals, including in the combined radicals such as alkoxy, haloalkyl, etc., mean, for example, methyl, ethyl, n- or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

Alkenyl especially also includes straight-chain or branched open-chain hydrocarbyl radicals having more than one double bond, such as 1,3-butadienyl and 1,4-pentadienyl, but also allenyl or cumulenyl radicals having one or more cumulated double bonds, for example allenyl(1,2-propadienyl), 1,2-butadienyl and 1,2,3-pentatrienyl. Alkenyl means, for example, vinyl which may optionally be substituted by further alkyl radicals, for example prop-1-en-1-yl, but-1-en-1-yl, allyl, 1-methylprop-2-en-1-yl, 2-methylprop-2-en-1-yl, but-2-en-1-yl, 1-methylbut-3-en-1-yl and 1-methylbut-1-yl, 2-methylprop-1-en-1-yl, 1-methylprop-1-en-1-yl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl, pentenyl, 2-methylpentenyl or hexenyl.

Alkynyl especially also includes straight-chain or branched open-chain hydrocarbyl radicals having more than one triple bond or else having one or more triple bonds and one or more double bonds, for example 1,3-butatrienyl or 3-penten-1-yn-1-yl. (C₂-C₆)-Alkynyl means, for example, ethynyl, propargyl, 1-methylprop-2-yn-1-yl, 2-butynyl, 2-pentynyl or 2-hexynyl, preferably propargyl, but-2-yn-1-yl, but-3-yn-1-yl or 1-methylbut-3-yn-1-yl.

The term “cycloalkyl” means a carbocyclic, saturated ring system having preferably 3-8 ring carbon atoms, e.g. cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl(norbornyl), bicyclo[2.2.2]octan-2-yl, adamantan-1-yl and adamantan-2-yl. The expression “(C₃-C₇)-cycloalkyl” means a brief notation for cycloalkyl having three to 7 carbon atoms, according to the range stated for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

Cycloalkenyl means a carbocyclic, nonaromatic, partially unsaturated ring system having preferably 4-8 carbon atoms, for example 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “aryl” means a mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphthyl, indenyl, indanyl, fluorenyl, biphenylyl, where the bonding site is on the aromatic system.

In systematic terms, “aryl” is generally also encompassed by the term “optionally substituted phenyl”.

According to the invention, “heteroaryl” represents heteroaromatic compounds, i.e. fully unsaturated aromatic heterocyclic compounds, preferably 5- to 7-membered rings having 1 to 3, preferably 1 or 2, identical or different heteroatoms, preferably O, S or N. Inventive heteroaryls are, for example, furyl, thienyl, pyrazolyl, imidazolyl, 1,2,3- and 1,2,4-triazolyl, isoxazolyl, thiazolyl, isothiazolyl, 1,2,3-, 1,3,4-, 1,2,4- and 1,2,5-oxadiazolyl, azepinyl, pyrrolyl, pyridyl, pyridazinyl, pyrimidinyl, pyrazinyl, 1,3,5-, 1,2,4- and 1,2,3-triazinyl, 1,2,4-, 1,3,2-, 1,3,6- and 1,2,6-oxazinyl, oxepinyl, thiepinyl, 1,2,4-triazolonyl and 1,2,4-diazepinyl. The inventive heteroaryl groups may also be substituted by one or more identical or different radicals.

Alkoxy means an alkyl radical bonded via an oxygen atom, alkenyloxy means an alkenyl radical bonded via an oxygen atom, alkynyloxy means an alkynyl radical bonded via an oxygen atom, cycloalkyloxy means a cycloalkyl radical bonded via an oxygen atom and cycloalkenyloxy means a cycloalkenyl radical bonded via an oxygen atom.

According to the invention, “alkylthio”—alone or as part of a chemical group—is straight-chain or branched S-alkyl, preferably having 1 to 8 or having 1 to 6 carbon atoms, for example methylthio, ethylthio, n-propylthio, isopropylthio, n-butylthio, isobutylthio, sec-butylthio and tert-butylthio. Alkenylthio means an alkenyl radical bonded via a sulfur atom, alkynylthio means an alkynyl radical bonded via a sulfur atom, cycloalkylthio means a cycloalkyl radical bonded via a sulfur atom and cycloalkenylthio means a cycloalkenyl radical bonded via a sulfur atom.

According to the invention, “alkylsulfinyl”—alone or as part of a chemical group—is straight-chain or branched alkylsulfinyl, preferably having 1 to 8 or having 1 to 6 carbon atoms, for example methylsulfinyl, ethylsulfinyl, n-propylsulfinyl, isopropylsulfinyl, n-butylsulfinyl, isobutylsulfinyl, sec-butylsulfinyl and tert-butylsulfinyl.

According to the invention, “alkylsulfonyl” - alone or as part of a chemical group—is straight-chain or branched alkylsulfonyl, preferably having 1 to 8 or having 1 to 6 carbon atoms, for example methylsulfonyl, ethylsulfonyl, n-propylsulfonyl, isopropylsulfonyl, n-butylsulfonyl, isobutylsulfonyl, sec-butylsulfonyl and tert-butylsulfonyl.

According to the invention, “cycloalkylsulfonyl”—alone or as part of a chemical group—is optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “arylsulfonyl” is optionally substituted phenylsulfonyl or optionally substituted polycyclic arylsulfonyl, for example substituted by halogen, alkyl, haloalkyl, haloalkoxy or alkoxy groups.

The term “sulfilimine” represents a group with a nitrogen-sulfur double bond, in which nitrogen and sulfur have further substitution, the nitrogen atom preferably by a further-substituted carbonyl group and the sulfur preferably by two identical or mixed alkyl, aryl and cycloalkyl substituents, for example in the form of an N-(di-n-butylsulfanylidene), N-(diisopropylsulfanylidene), N-(di-n-propylsulfanylidene), N-(di-n-pentylsulfanylidene), N-(diisobutylsulfanylidene), N-(cyclobutylisopropylsulfanylidene), N-(n-propylisopropylsulfanylidene), N-(cyclopropylisopropylsulfanylidene) or N-(isobutylisopropylsulfanylidene) unit.

According to the type and linkage of the substituents, the compounds of the formula (I) may be present as stereoisomers. The possible stereoisomers defined by the specific three-dimensional form thereof, such as enantiomers, diastereomers, Z and E isomers, are all encompassed by the formula (I). When, for example, one or more alkenyl groups are present, diastereomers (Z and E isomers) may occur. When, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomer excess, or else on the preparative scale to produce test specimens for biological testing. It is likewise possible to selectively prepare stereoisomers by using stereoselective reactions with use of optically active starting materials and/or assistants. The invention thus also relates to all stereoisomers which are encompassed by the formula (I) but are not shown with their specific stereoisomeric form, and mixtures thereof.

The radical definitions stated above, in general terms or listed within areas of preference, apply both to the end products of the formula (I) and correspondingly to the starting materials and intermediates required for the preparation in each case. These radical definitions can be exchanged with one another, i.e. including combinations between the preferred ranges stated.

The term “useful plants” as used here refers to crop plants which are employed as plants for obtaining foods, animal feeds or for industrial purposes.

Synthesis:

Fluoroalkyl-substituted 2-amidobenzimidazoles can be prepared by known processes (cf. J. Med. Chem. 2000, 43, 4084; Bioorg. Med. Chem. 2008, 16, 6965; Bioorg. Med. Chem. 2008, 16, 3955; Org. Proc. Res. Develop. 2007, 11, 693; J. Med. Chem. 2009, 52, 514; J. Heterocyclic Chem. 2001, 38, 979; WO2000026192; WO2003106430; WO9704771; WO2000029384, WO2000032579). Various literature preparation routes were used to form the core structure, and some were optimized (see scheme 1). Selected detailed synthesis examples are detailed in the next section. The synthesis routes used and examined proceed from commercially available or easily preparable 2-amino-3-nitrobenzoic acids or 2,3-diaminobenzonitriles.

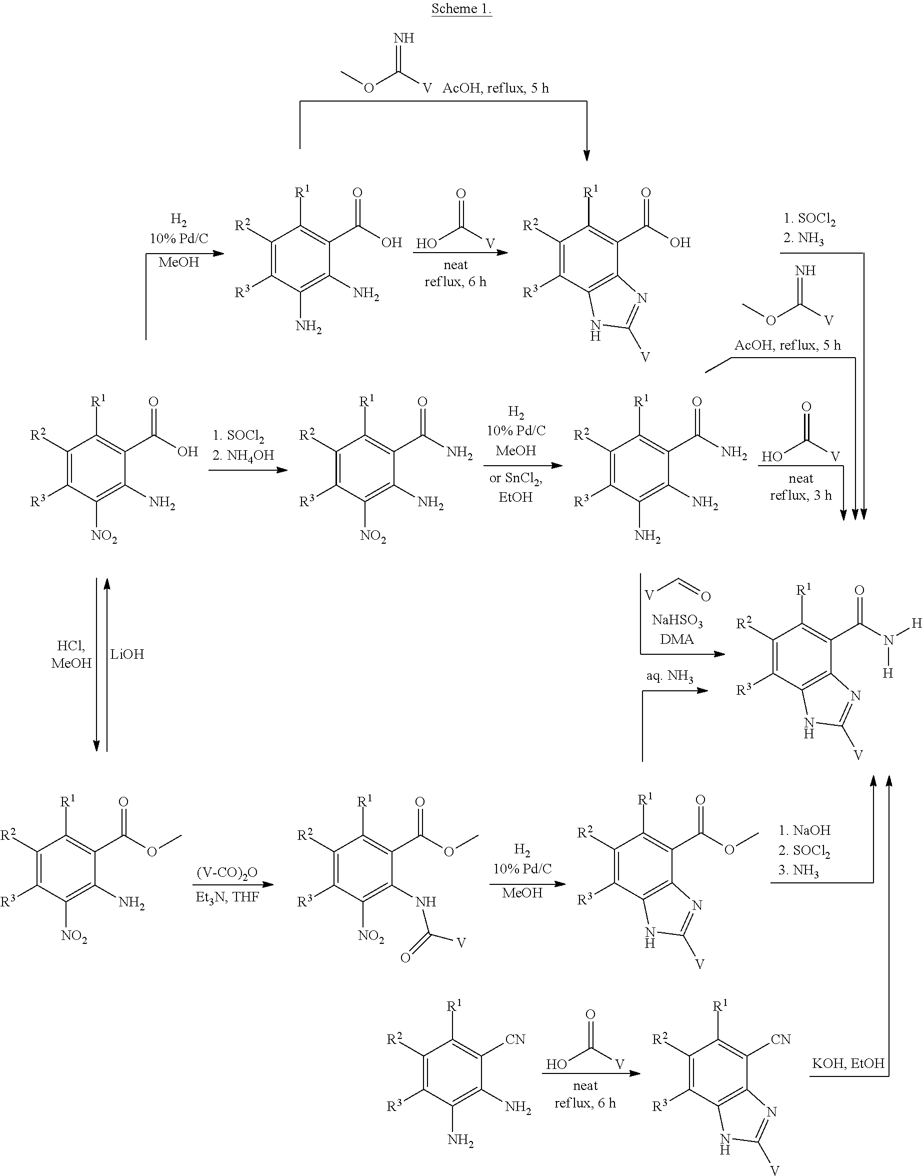

The relevant 2-amino-3-nitrobenzoic acid with optional additional substitution can be converted with the aid of thionyl chloride and ammonia to the corresponding 2-amino-3-nitrobenzamide, which is reduced either with hydrogen in the presence of palladium on carbon in a suitable solvent or with tin(II) chloride to give an optionally further-substituted 2,3-diaminobenzamide. The 2,3-diaminobenzamide thus obtained can be converted in the subsequent step via various reaction variants, for example condensation with a carboxylic acid, with an aldehyde or an amide oxime, to the desired benzimidazole derivative. Alternatively, the corresponding benzimidazole can also be formed by condensation of a 2,3-diaminobenzoic acid with a carboxylic acid or by N-acylation of a 2-amino-3-nitrobenzoic ester and subsequent reduction with hydrogen in the presence of palladium on carbon, and the carboxyl function can be converted to the desired amide in the final step. A further reaction route to the synthesis of the inventive compounds is the condensation of an optionally substituted 2,3-diaminobenzonitrile with a corresponding carboxylic acid and the subsequent reaction with a hydroxide base (e.g. potassium hydroxide) in a protic solvent (e.g. ethanol).

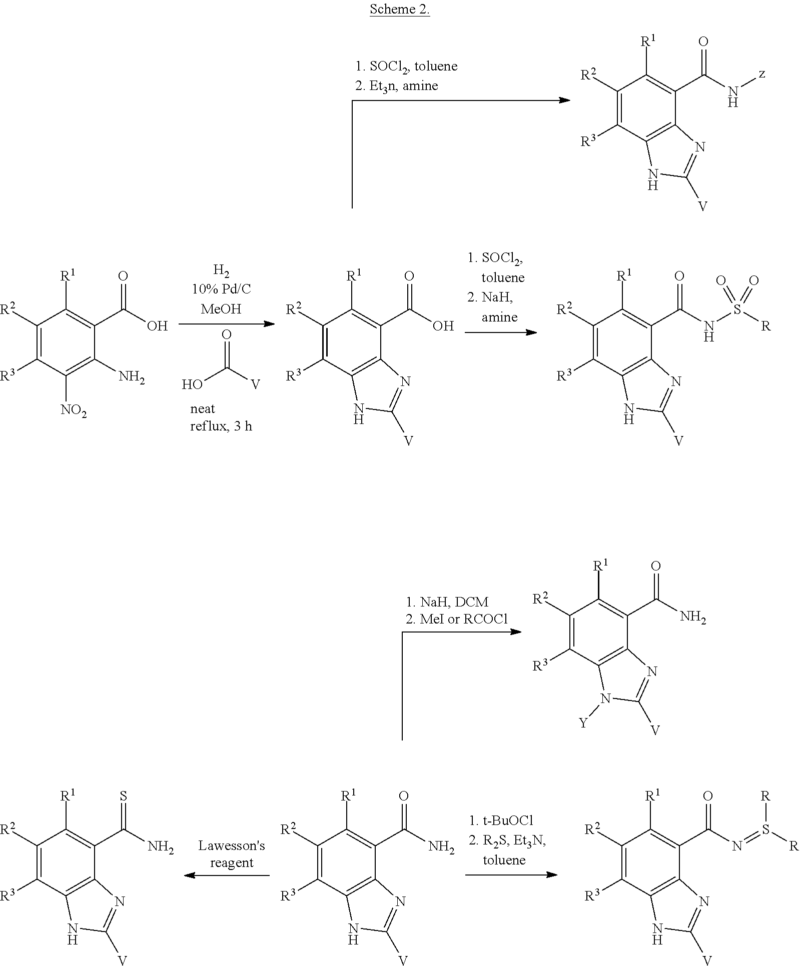

The resulting carboxyl-substituted benzimidazoles can be converted with the aid of thionyl chloride in a suitable solvent and subsequent reaction with a substituted amine or a substituted sulfonamide to correspondingly N-substituted benzimidazoles. The functionalization of a benzimidazole nitrogen atom is possible by deprotonation with a suitable base, for example sodium hydride in an aprotic solvent, and subsequent reaction with a suitable electrophile, for example an acyl chloride, an alkyl halide or a chloroformate. The amide group of the fluoroalkyl-substituted 2-amidobenzimidazoles prepared in accordance with the invention can also be converted to the corresponding thioamide with the aid of 2,4-bis(4-methoxyphenyl)-1,3,2,4-dithiadiphosphetane 2,4-disulfide, or to the corresponding substituted sulfilimines in a two-stage synthesis by reaction with tert-butyl hypochlorite and AIBN in an aprotic solvent (e.g. carbon tetrachloride) and subsequent reaction with a dialkyl sulfide in the presence of a base (e.g. triethylamine) in a suitable solvent (e.g. toluene) (see scheme 2). The preparation and the use of the inventive compounds is illustrated by the examples which follow.

The ¹H NMR, ¹³C NMR and ¹⁹F NMR spectroscopy data reported for the chemical examples described in the paragraphs which follow (400 MHz in the case of ¹H NMR and 150 MHz in the case of ¹³C NMR and 375 MHz in the case of ¹⁹F NMR, solvent: CDCl₃, CD₃OD or d₆-DMSO, internal standard: tetramethylsilane δ=0.00 ppm), were obtained with a Bruker instrument, and the signals identified are defined as follows: br=broad; s=singlet, d=doublet, t=triplet, dd=double doublet, ddd=doublet of a double doublet, m=multiplet, q=quartet, quint=quintet, sext=sextet, sept=septet, t=triplet, dq=double quartet, dt=double triplet, tt=triple triplet

### SYNTHESIS EXAMPLES

### No. 1-1: 2-[chloro(difluoro)methyl]-1H-benzimidazole-4-carboxamide

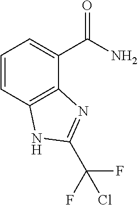

2-Amino-3-nitrobenzoic acid (3.00 g, 16.47 mmol) was dissolved in dimethoxyethane (15 ml), thionyl chloride was added (2.61 g, 21.91 mmol), and the mixture was stirred at 50° C. for 12 h. Subsequently, the reaction mixture was concentrated under reduced pressure, the solvent was removed and then toluene was added, and the mixture was concentrated again. Thereafter, aqueous saturated ammonia solution (40 ml) was initially charged in a round-bottom flask and cooled to 10° C., and the acid chloride (3.30 g, 16.45 mmol) from the first step, which had not been purified any further, was added dropwise while stirring vigorously. In the course of this, the temperature of the reaction mixture was kept below 40° C. After the end of the addition, the reaction mixture was stirred at 50° C. for one hour, then diluted with water and stirred at room temperature. By filtering off the orange precipitate which formed with suction, washing with water and drying, 2-amino-3-nitrobenzamide (2.60 g, 87%) was obtained. 2-Amino-3-nitrobenzamide (2.60 g, 14.35 mmol) was then added in a metal vessel to palladium on carbon (water-moist catalyst, 10% Pd, 0.05 equiv., 0.072 mmol) in methanol (80 ml). In a laboratory reactor, hydrogen was introduced into the metal vessel and the resulting reaction mixture was stirred at room temperature at a pressure of 2 bar for 5 h. After complete conversion, the catalyst was filtered off through Celite and washed with methanol. The filtrate was concentrated under reduced pressure and the residue was extracted with water and ethyl acetate. The combined organic phases were dried over magnesium sulfate, filtered and concentrated under reduced pressure. The resulting crude product did not need any further purification and contained 2,3-diaminobenzamide (2.15 g, 98%) in a purity of >95%. 2,3-Diaminobenzamide (200 mg, 1.32 mmol) was dissolved in chlorodifluoroacetic acid (3 ml) with vigorous stirring. The resulting reaction mixture was subsequently stirred under reflux for 3 h. After cooling to room temperature, NaHCO₃ solution was added. In the course of this, the 2-[chloro(difluoro)methyl]-1H-benzimidazole-4-carboxamide target product (190 mg, 58%) precipitated out as a colorless solid, which was filtered off with suction, washed with water and finally dried. ¹H NMR (400 MHz, d₆-DMSO δ, ppm) 14.40 (br. s, 1H, NH), 8.72 (br. s, 1H, NH), 8.02 (m, 1H, NH), 7.92 (m, 1H), 7.74 (m, 1H), 7.55 (m, 1H); ¹³C NMR (150 MHz, d₆-DMSO δ, ppm) 165.5, 147.9, 139.1, 134.2, 124.9, 124.5, 123.9, 118.4-122.2 (t, CF₂Cl), 116.6.

### No. 1-2: 2-(pentafluoroethyl)-1H-benzimidazole-4-carboxamide

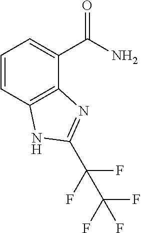

Methyl 2-amino-3-nitrobenzoate (1.30 g, 6.63 mmol) was dissolved in abs. THF (tetrahydrofuran) (10 ml), triethylamine (2.77 ml, 19.88 mmol) was added and the mixture was stirred at room temperature under argon for 20 min. Thereafter, a solution of pentafluoropropionic anhydride (3.93 ml, 19.88 mmol) in abs. THF (5 ml) was slowly added dropwise and the reaction mixture was stirred at room temperature for 4 h. After the addition of water, the aqueous phase was extracted repeatedly with ethyl acetate. The combined organic phases were then extracted once again with water, dried over magnesium sulfate, filtered and concentrated. By column chromatography purification of the resulting crude product, 3-nitro-2-[(2,2,3,3,3-pentafluoropropanoyl)amino]benzamide (1.80 g, 79%) was isolated. 3-Nitro-2-[(2,2,3,3,3-pentafluoropropanoyl)amino]benzamide (1.80 g, 5.26 mmol) was then dissolved in methanol (50 ml) and added in a metal vessel to palladium on carbon (water-moist catalyst, 10% Pd, 0.02 equiv., 84 mg, 0.079 mmol) in methanol (30 ml). In a laboratory reactor, hydrogen was introduced into the metal vessel and the resulting reaction mixture was stirred at room temperature at a pressure of 2 bar for 5 h. After complete conversion, the catalyst was filtered off through Celite and washed with methanol. The solvent was carefully distilled out of the filtrate under reduced pressure and the residue was purified by column chromatography (silica gel, gradient with n-heptane and ethyl acetate). This gave methyl 2-(pentafluoroethyl)-1H-benzimidazole-7-carboxylate (800 mg, 49%), which in the next step was partially dissolved in THF (1 ml), and water (7 ml) and sodium hydroxide (163 mg, 4.08 mmol) were added. The resulting reaction mixture was stirred under reflux for 3 h. After cooling to room temperature, a pH of 2-3 was established by adding dil. HCl and the precipitate formed was filtered off with suction, washed with heptane and dried. In this way, 2-(pentafluoroethyl)-1H-benzimidazole-7-carboxylic acid (570 mg, 75%) was obtained, which was then dissolved in dichloromethane (6 ml), and oxalyl chloride (0.15 ml, 1.73 mmol) and a catalytic amount of N,N-dimethylformamide were added. The reaction mixture was stirred at room temperature for 15 min and then at 70° C. for 2 h, and thereafter was concentrated completely. After addition of toluene, the mixture was concentrated again and the acid chloride thus obtained (600 mg, 2.01 mmol), without further purification, was dissolved in dioxane (6 ml). Then ammonia (g) was introduced while cooling and the mixture was stirred at room temperature for 1 h. The reaction mixture was concentrated completely and the residue was purified by column chromatography (silica gel, gradient with n-heptane and ethyl acetate). This gave 2-(pentafluoroethyl)-1H-benzimidazole-4-carboxamide (560 mg, 95%). ¹H NMR (400 MHz, d₆-DMSO δ, ppm) 14.32 (br. s, 1H, NH), 8.74 (br. s, 1H, NH), 7.95 (d, 1H), 7.86 (d, 1H), 7.68 (br s, 1H, NH), 7.45 (dd, 1H); ¹⁹F NMR (375 MHz, d₆-DMSO δ, ppm) −114.6, −83.3.

### Example No. 1-3

### N-ethyl-2-(trifluoromethyl)-1H-benzimidazole-4-carboxamide

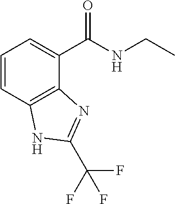

2-Trifluoromethyl-1H-benzimidazole-7-carboxylic acid (200 mg, 0.87 mmol) was dissolved in dichloromethane (6 ml), and oxalyl chloride (0.15 ml, 1.73 mmol) and a catalytic amount of N,N-dimethylformamide were added. The reaction mixture was stirred at room temperature for 15 min and then at 70° C. for 2 h, and thereafter was concentrated completely. After addition of toluene, the mixture was concentrated again and the acid chloride thus obtained (210 mg, 0.85 mmol), without further purification, was dissolved in tetrahydrofuran (1 ml) and added dropwise to a solution of ethylamine (0.46 ml, 0.93 mmol) and triethylamine (0.18 ml, 1.27 mmol) in tetrahydrofuran (3 ml). The reaction mixture was stirred at room temperature for 3 h, then water was added and the mixture was extracted repeatedly with ethyl acetate. The combined organic phases were dried over magnesium sulfate, filtered and concentrated. By column chromatography purification of the resulting residue (silica gel, gradient of ethyl acetate and n-heptane), N-ethyl-2-(trifluoromethyl)-1H-benzimidazole-4-carboxamide was isolated as a colorless solid (50 mg, 22%). ¹H NMR (400 MHz, CDCl₃ δ, ppm) 9.41 (br. s, 1H, NH), 8.02 (d, 1H), 7.55 (m, 1H), 7.43 (m, 1H), 6.42 (br. s, 1H, NH), 3.60 (q, 2H), 1.34 (t, 3H).

### Ex. No. 1-4

### 1-(2-methylpropanoyl)-2-(trifluoromethyl)benzimidazole-4-carboxamide

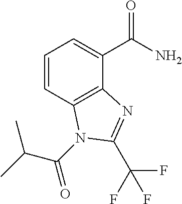

2-(Trifluoromethyl)-1H-benzimidazole-4-carboxamide (120 mg, 0.52 mmol) was dissolved in N,N-dimethylformamide and cooled to 5° C., and sodium hydride (25 mg, 0.63 mmol, 60% dispersion in oil) was added. Stirring at room temperature under argon for 20 min was followed by the addition of isobutyryl chloride (61 mg, 0.58 mmol). The resulting reaction mixture was stirred at room temperature for 4 h, then water was added and the mixture was extracted repeatedly with ethyl acetate. The combined organic phases were dried over magnesium sulfate, filtered and concentrated. By column chromatography purification of the resulting residue (silica gel, gradient of ethyl acetate and n-heptane), 1-(2-methylpropanoyl)-2-(trifluoromethyl)-1H-benzimidazole-4-carboxamide was isolated as a colorless solid (30 mg, 18%). ¹H NMR (400 MHz, CDCl₃ δ, ppm) 9.42 (br. s, 1H, NH), 8.21 (d, 1H), 7.97 (br. s, 1H, NH), 7.84 (d, 1H), 7.59 (dd, 1H), 3.00 (sept, 1H), 1.38 (d, 6H).

### Ex. No. 1-5

### N-(dibutyl-lambda⁴-sulfanylidene)-2-(trifluoromethyl)-1H-benzimidazole-4-carboxamide

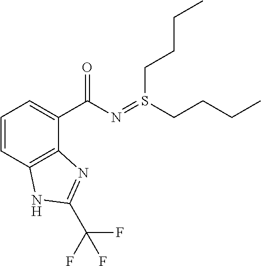

2-(Trifluoromethyl)-1H-benzimidazole-4-carboxamide (1000 mg, 4.36 mmol) was dissolved in tetrachloromethane (7 ml) and, after stirring at room temperature for 5 min, heated to reflux. Thereafter, 2,2′-azobis-2-methylpropanenitrile (29 mg, 0.18 mmol) was added and the mixture was stirred for 5 min. tert-Butyl hypochlorite (569 mg, 5.24 mmol) is likewise dissolved in tetrachloromethane (3 ml) and cautiously added in portions to the refluxing solution. The resulting reaction mixture was stirred under reflux for 3 h. After cooling to room temperature, the precipitate formed was filtered off with suction, washed with a little tetrachloromethane and dried under reduced pressure. A portion of the colorless solid obtained (200 mg, 0.76 mmol) was then dissolved in toluene (4 ml) and stirred for 5 min, and dibutyl sulfide (0.12 ml, 0.84 mmol) and triethylamine (0.12 ml, 0.84 mmol) were added. The resulting reaction mixture was stirred at room temperature for 4 h and then partially concentrated under reduced pressure. In the course of this, N-(dibutyl-lambda⁴-sulfanylidene)-2-(trifluoromethyl)-1H-benzimidazole-4-carboxamide precipitates out as a colorless solid (150 mg, 57%); ¹H NMR (400 MHz, CDCl₃ δ, ppm) 11.41 (br. s, 1H, NH), 8.12 (d, 1H), 7.95 (d, 1H), 7.38 (dd, 1H), 3.15 (m, 2H), 2.99 (m, 2H), 1.79 (m, 4H), 1.51 (m, 4H), 0.97 (t, 6H).

### Ex. No. 1-6

### 2-[difluoro(methoxy)methyl]-1H-benzimidazole-4-carboxamide

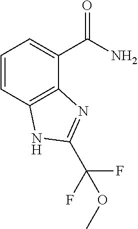

2-(Chloro(difluoro)methyl)-1H-benzimidazole-4-carboxamide (200 mg, 0.81 mmol) was dissolved under argon in abs. N,N-dimethylformamide (4 ml), and sodium n-propoxide (668 mg, 1.63 mmol) was added. The resulting reaction mixture was stirred at 80° C. for 6 h and, after cooling to room temperature, water and ethyl acetate were added. The aqueous phase was extracted repeatedly with ethyl acetate and the combined organic phases were dried over magnesium sulfate, filtered and concentrated. The resulting residue was purified by column chromatography and gave 2-[difluoro(methoxy)methyl]-1H-benzimidazole-4-carboxamide (30 mg, 14%) as a colorless solid; ¹H NMR (400 MHz, CD₃OD δ, ppm) 8.06 (d, 1H), 7.77 (d, 1H), 7.48 (dd, 1H), 3.85 (s, 3H).

### Example No. 2-14

### 6-chloro-2-(trifluoromethyl)-1H-benzimidazole-4-carboxamide

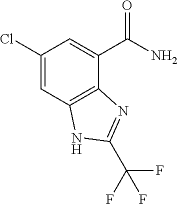

Methyl 2-amino-5-chloro-3-nitrobenzoate (1.0 g, 4.34 mmol) was dissolved in water (5 ml), lithium hydroxide (2.5 equiv.) was added and the mixture was stirred under reflux conditions for 1 h. After cooling to room temperature, the reaction solution was acidified to pH 2 with conc. HCl and the resulting precipitate was filtered off with suction, washed and dried. The 2-amino-5-chloro-3-nitrobenzoic acid thus obtained (0.90 g, 4.15 mmol), without further purification, was dissolved in dimethoxyethane (5 ml), thionyl chloride was added (0.40 ml, 5.53 mmol), and the mixture was stirred at 50° C. for 12 h. Subsequently, the reaction mixture was concentrated under reduced pressure, the solvent was removed and then toluene was added, and the mixture was concentrated again. Thereafter, aqueous saturated ammonia solution (40 ml) was initially charged in a round-bottom flask and cooled to 10° C., and the acid chloride (0.95 g, 4.04 mmol) from the first step, which had not been purified any further, was added dropwise while stirring vigorously. In the course of this, the temperature of the reaction mixture was kept below 40° C. After the end of the addition, the reaction mixture was stirred at 50° C. for one hour, then diluted with water and stirred at room temperature. By filtering off the precipitate which formed with suction, washing with water and drying, 2-amino-5-chloro-3-nitrobenzamide (0.57 g, 65%) was obtained. 2-Amino-5-chloro-3-nitrobenzamide (0.57 g, 2.64 mmol) was dissolved in ethanol, tin(II) chloride dihydrate (2.15 g, 9.52 mmol) was added and the mixture was stirred under reflux for 4 h. After cooling to room temperature, the reaction solution was concentrated under reduced pressure and added to water, and sat. NaHCO₃ solution was used to adjust it to a pH of 8. The aqueous phase was extracted repeatedly with ethyl acetate, and the combined organic phases were dried over magnesium sulfate, filtered and concentrated. By column chromatography purification of the crude product (silica gel and gradient of n-heptane and ethyl acetate), 2,3-diamino-5-chlorobenzamide (360 mg, 73%) was obtained. 2,3-Diamino-5-chlorobenzamide (100 mg, 0.54 mmol) was dissolved in trifluoroacetic acid (1.5 ml) while stirring vigorously. The resulting reaction mixture was subsequently stirred under reflux for 3 h. After cooling to room temperature, NaHCO₃ solution was added. In the course of this, the 6-chloro-2-(trifluoromethyl)-1H-benzimidazole-4-carboxamide target product (110 mg, 77%) precipitated out as a colorless solid, which was filtered off with suction, washed with water and finally dried. ¹H NMR (400 MHz, d₆-DMSO δ, ppm) 14.05 (br. s, 1H, NH), 8.49 (br. s, 1H, NH), 8.04 (d, 1H), 7.95 (br. s, 1H, NH), 7.90 (m, 1H).

### Example No. 3-7

### 2-(difluoromethyl)-6-(trifluoromethyl)-1H-benzimidazole-4-carboxamide

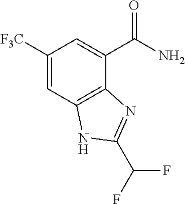

2,3-Diamino-5-trifluoromethylbenzonitrile (250 mg, 1.24 mmol) was dissolved in difluoroacetic acid (3.0 ml) while stirring vigorously. The resulting reaction mixture was subsequently stirred under reflux for 6 h. After cooling to room temperature, NaHCO₃ solution was added. In the course of this, the 2-(difluoromethyl)-6-(trifluoromethyl)-1H-benzimidazole-4-nitrile target product (320 mg, 98%) precipitated out as a solid, which was filtered off with suction, washed with water and finally dried. 2-(Difluoromethyl)-6-(trifluoromethyl)-1H-benzimidazole-4-nitrile (250 mg, 0.96 mmol) was dissolved in warm ethanol (3 ml), potassium hydroxide (268 mg, 4.79 mmol) was added and the mixture was stirred under reflux for 4 h. After cooling to room temperature, the solvent was removed under reduced pressure and the residue was extracted with ethyl acetate and water. The water phase was extracted repeatedly with ethyl acetate, and the combined organic phases were dried over sodium sulfate, filtered and concentrated. The resulting residue was purified by column chromatography (ethyl acetate-heptane gradient) and gave 2-(difluoromethyl)-6-(trifluoromethyl)-1H-benzimidazole-4-carboxamide as a solid (40 mg, 14%). ¹H NMR (400 MHz, d₆-DMSO 8, ppm) 13.93 (br. s, 1H, NH), 8.81 (br. s, 1H, NH), 8.29 (s, 1H), 8.14 (s, 1H), 7.80 (br. s, 1H, NH), 7.31-7.03 (t, 1H).

### Example No. 4-14

### 6-methyl-2-(trifluoromethyl)-1H-benzimidazole-4-carboxamide

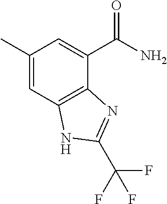

2-Amino-5-methyl-3-nitrobenzoic acid (2.00 g, 10.19 mmol) was dissolved in dimethoxyethane (15 ml), thionyl chloride was added (0.99 ml, 13.56 mmol), and the mixture was stirred at 50° C. for 12 h. Subsequently, the reaction mixture was concentrated under reduced pressure, the solvent was removed and then toluene was added, and the mixture was concentrated again. Thereafter, aqueous saturated ammonia solution (40 ml) was initially charged in a round-bottom flask and cooled to 10° C., and the acid chloride (2.20 g, 10.19 mmol) from the first step, which had not been purified any further, was added dropwise while stirring vigorously. In the course of this, the temperature of the reaction mixture was kept below 40° C. After the end of the addition, the reaction mixture was stirred at 50° C. for one hour, then diluted with water and stirred at room temperature. By filtering off the orange precipitate which formed with suction, washing with water and drying, 2-amino-5-methyl-3-nitrobenzamide (2.01 g, 99%) was obtained. 2-Amino-5-methyl-3-nitrobenzamide (1.00 g, 5.12 mmol) was dissolved in ethanol, tin(II) chloride dihydrate (4.16 g, 18.45 mmol) was added and the mixture was stirred under reflux for 4 h. After cooling to room temperature, the reaction solution was concentrated under reduced pressure and added to water, and sat. NaHCO₃ solution was used to adjust it to a pH of 8. The aqueous phase was extracted repeatedly with ethyl acetate, and the combined organic phases were dried over magnesium sulfate, filtered and concentrated. By column chromatography purification of the crude product (silica gel and gradient of n-heptane and ethyl acetate), 2,3-diamino-5-methylbenzamide (270 mg, 32%) was obtained. 2,3-Diamino-5-methylbenzamide (250 mg, 1.51 mmol) was dissolved in trifluoroacetic acid (3 ml) while stirring vigorously. The resulting reaction mixture was subsequently stirred under reflux for 4 h. After cooling to room temperature, NaHCO₃ solution was added. In the course of this, the 6-methyl-2-(trifluoromethyl)-1H-benzimidazole-4-carboxamide target product (340 mg, 88%) precipitated out as a colorless solid, which was filtered off with suction, washed with water and finally dried. ¹H NMR (400 MHz, d₆-DMSO δ, ppm) 14.38 (br. s, 1H, NH), 8.60 (br. s, 1H, NH), 7.81 (m, 1H, NH), 7.78 (m, 1H), 7.62 (m, 1H), 2.48 (s, 3H); ¹⁹F NMR (375 MHz, CD₃OD δ, ppm) −65.8.

In analogy to the preparation examples adduced above, and taking account of the general information regarding the preparation of fluoroalkyl-substituted 2-amidobenzimidazoles of the formula (I), the following compounds are obtained:

TABLE 1
(I)

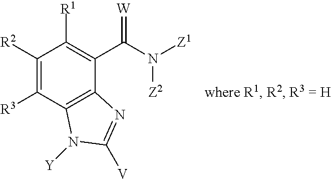

| No. | V | W | Y | Z¹ | Z² |
|---|---|---|---|---|---|
| 1-7 | 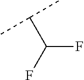 | O | H | H | H |
| 1-8 | 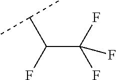 | O | H | H | H |
| 1-9 | 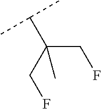 | O | H | H | H |
| 1-10 | 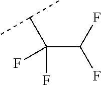 | O | H | H | H |
| 1-11 | 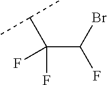 | O | H | H | H |
| 1-12 | 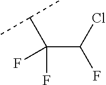 | O | H | H | H |
| 1-13 | 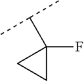 | O | H | H | H |
| 1-14 | 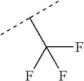 | O | H | H | H |
| 1-15 | 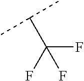 | O | H | 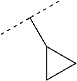 | H |
| 1-16 | 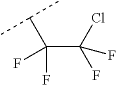 | O | H | H | H |
| 1-17 | 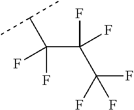 | O | H | H | H |
| 1-18 | 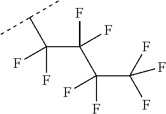 | O | H | H | H |
| 1-19 | 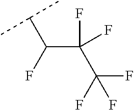 | O | H | H | H |
| 1-20 | 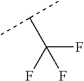 | O | H | 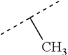 | H |
| 1-21 | 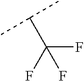 | O | H | 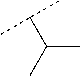 | H |
| 1-22 | 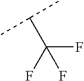 | O | H | 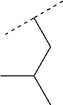 | H |
| 1-23 | 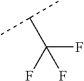 | O | H | 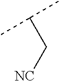 | H |
| 1-24 | 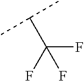 | O | H | 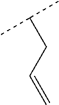 | H |
| 1-25 | 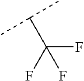 | O | H | 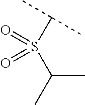 | H |
| 1-26 | 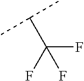 | O | H | 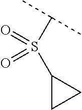 | H |
| 1-27 | 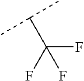 | O | 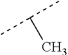 | H | H |
| 1-28 | 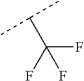 | O | 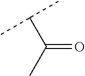 | H | H |
| 1-29 | 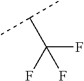 | O | 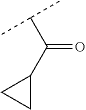 | H | H |
| 1-30 | 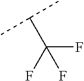 | O | 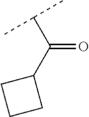 | H | H |
| 1-31 | 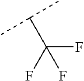 | O | 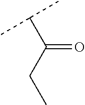 | H | H |
| 1-32 | 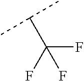 | O | 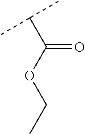 | H | H |
| 1-33 | 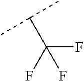 | O | H | 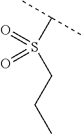 | H |
| 1-34 | 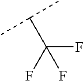 | O | H | 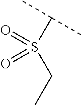 | H |
| 1-35 | 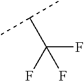 | O | H | 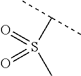 | H |
| 1-36 | 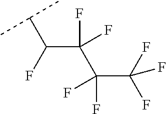 | O | H | H | H |
| 1-37 | 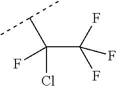 | O | H | H | H |
| 1-38 | 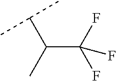 | O | H | H | H |
| 1-39 | 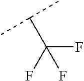 | S | H | H | H |
| 1-40 | 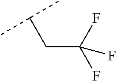 | O | H | H | H |
| 1-41 | 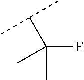 | O | H | H | H |
| 1-42 | 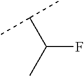 | O | H | H | H |
| 1-43 | 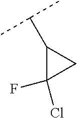 | O | H | H | H |
| 1-44 | 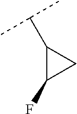 | O | H | H | H |
| 1-45 | 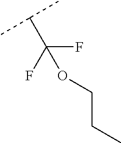 | O | H | H | H |
| 1-46 | 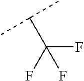 | O | H | 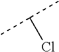 | H |
| 1-47 | 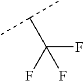 | O | H | 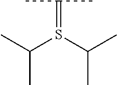 | |
| 1-48 | 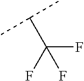 | O | H | 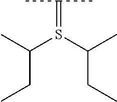 | |
| 1-49 | 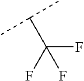 | O | H | 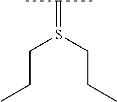 | |
| 1-50 | 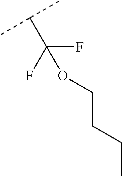 | O | H | H | H |
| 1-51 | 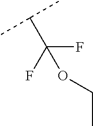 | O | H | H | H |
| 1-52 | 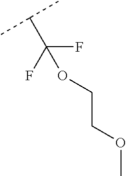 | O | H | H | H |
| 1-53 | 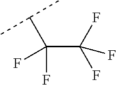 | O | 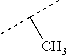 | H | H |
| 1-54 | 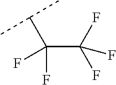 | O | 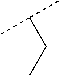 | H | H |
| 1-55 | 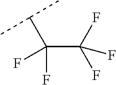 | O | 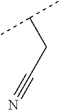 | H | H |
| 1-56 | 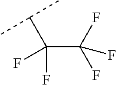 | O | 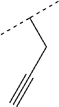 | H | H |
| 1-57 | 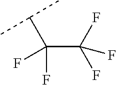 | O | 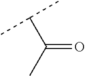 | H | H |
| 1-58 | 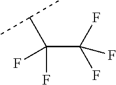 | O | 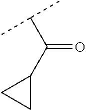 | H | H |
| 1-59 | 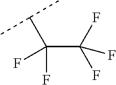 | O | 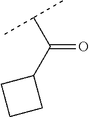 | H | H |
| 1-60 | 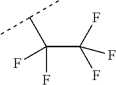 | O | 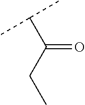 | H | H |
| 1-61 | 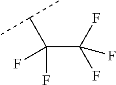 | O | 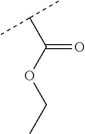 | H | H |
| 1-62 | 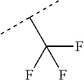 | O | 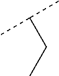 | H | H |
| 1-63 | 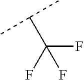 | O | 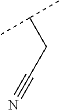 | H | H |
| 1-64 | 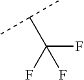 | O | 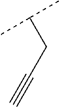 | H | H |
| 1-65 | 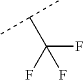 | O | 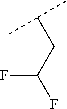 | H | H |
| 1-66 | 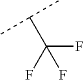 | O | 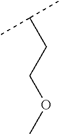 | H | H |
| 1-67 | 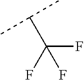 | O | H | 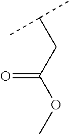 | H |
| 1-68 | 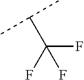 | O | H | 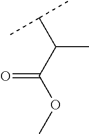 | H |
| 1-69 | 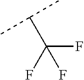 | O | 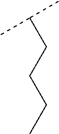 | H | H |
| 1-70 | 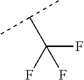 | O | 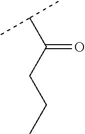 | H | H |
| 1-71 | 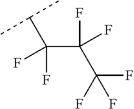 | O | 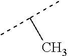 | H | H |
| 1-72 | 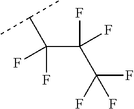 | O | 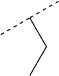 | H | H |
| 1-73 | 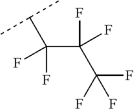 | O | 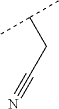 | H | H |
| 1-74 | 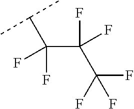 | O | 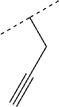 | H | H |
| 1-75 | 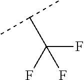 | O | 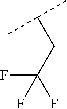 | H | H |
| 1-76 | 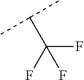 | O | 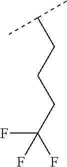 | H | H |
| 1-77 | 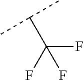 | O | 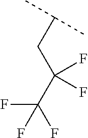 | H | H |
| 1-78 | 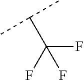 | O | 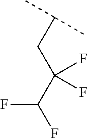 | H | H |
| 1-79 | 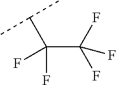 | O | 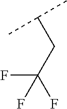 | H | H |
| 1-80 | 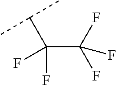 | O | 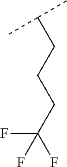 | H | H |
| 1-81 | 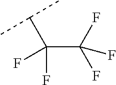 | O | 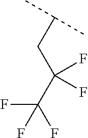 | H | H |
| 1-82 | 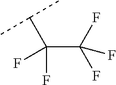 | O | 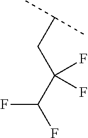 | H | H |
| 1-83 | 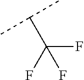 | O | H | 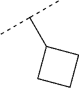 | H |
| 1-84 | 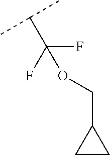 | O | H | H | H |
| 1-85 | 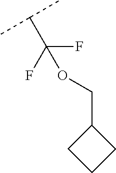 | O | H | H | H |
| 1-86 | 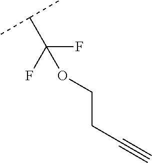 | O | H | H | H |
| 1-87 | 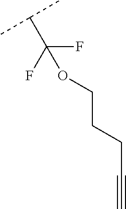 | O | H | H | H |
| 1-88 | 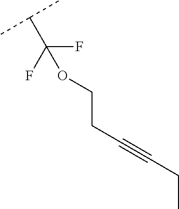 | O | H | H | H |
| 1-89 | 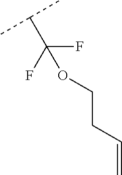 | O | H | H | H |
| 1-90 | 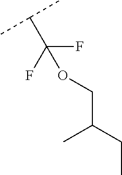 | O | H | H | H |
| 1-91 | 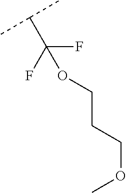 | O | H | H | H |
| 1-92 | 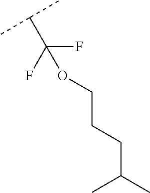 | O | H | H | H |
| 1-93 | 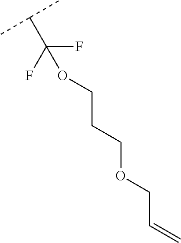 | O | H | H | H |
| 1-94 | 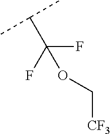 | O | H | H | H |
| 1-95 | 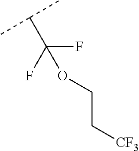 | O | H | H | H |
| 1-96 | 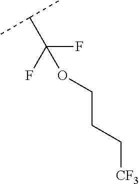 | O | H | H | H |
| 1-97 | 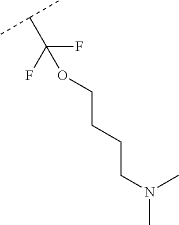 | O | H | H | H |
| 1-98 | 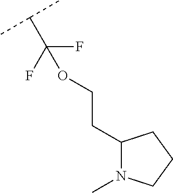 | O | H | H | H |
| 1-99 | 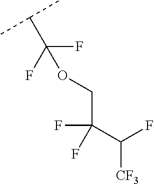 | O | H | H | H |
| 1-100 | 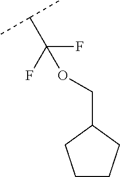 | O | H | H | H |
| 1-101 | 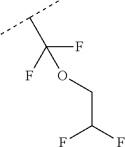 | O | H | H | H |
| 1-102 | 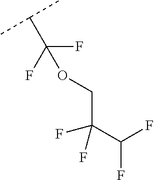 | O | H | H | H |
| 1-103 | 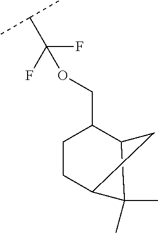 | O | H | H | H |

TABLE 2
(I)

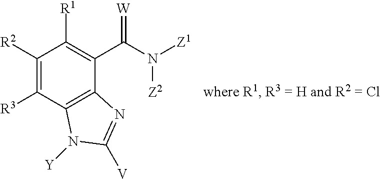

| No. | V | W | Y | Z¹ | Z² |
|---|---|---|---|---|---|
| 2-1 | 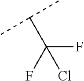 | O | H | H | H |
| 2-2 | 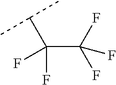 | O | H | H | H |
| 2-3 | 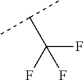 | O | H | 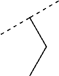 | H |
| 2-4 | 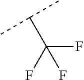 | O | 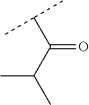 | H | H |
| 2-5 | 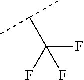 | O | H | 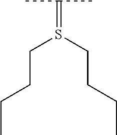 | |
| 2-6 | 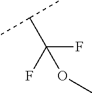 | O | H | H | H |
| 2-7 | 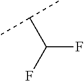 | O | H | H | H |
| 2-8 | 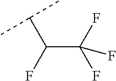 | O | H | H | H |
| 2-9 | 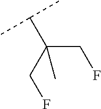 | O | H | H | H |
| 2-10 | 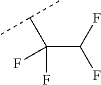 | O | H | H | H |
| 2-11 | 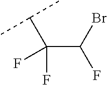 | O | H | H | H |
| 2-12 | 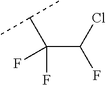 | O | H | H | H |
| 2-13 | 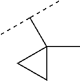 | O | H | H | H |
| 2-15 | 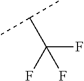 | O | H | 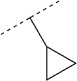 | H |
| 2-16 | 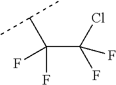 | O | H | H | H |
| 2-17 | 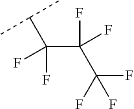 | O | H | H | H |
| 2-18 | 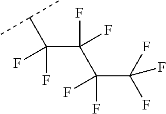 | O | H | H | H |
| 2-19 | 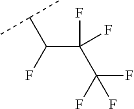 | O | H | H | H |
| 2-20 | 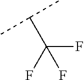 | O | H | 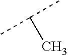 | H |
| 2-21 | 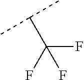 | O | H | 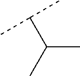 | H |
| 2-22 | 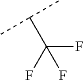 | O | H | 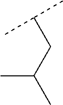 | H |
| 2-23 | 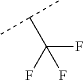 | O | H | 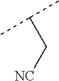 | H |
| 2-24 | 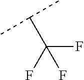 | O | H | 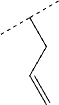 | H |
| 2-25 | 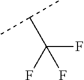 | O | H | 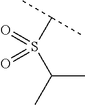 | H |
| 2-26 | 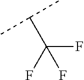 | O | H | 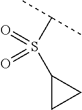 | H |
| 2-27 | 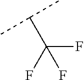 | O | 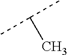 | H | H |
| 2-28 | 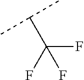 | O | 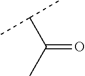 | H | H |
| 2-29 | 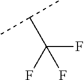 | O | 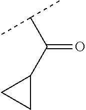 | H | H |
| 2-30 | 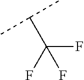 | O | 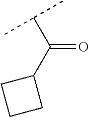 | H | H |
| 2-31 | 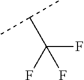 | O | 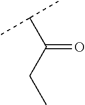 | H | H |
| 2-32 | 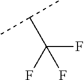 | O | 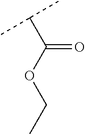 | H | H |
| 2-33 | 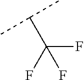 | O | H | 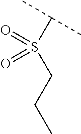 | H |
| 2-34 | 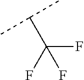 | O | H | 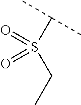 | H |
| 2-35 | 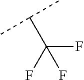 | O | H | 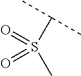 | H |
| 2-36 | 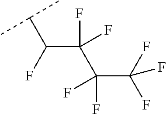 | O | H | H | H |
| 2-37 | 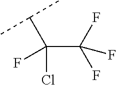 | O | H | H | H |
| 2-38 | 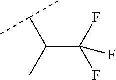 | O | H | H | H |
| 2-39 | 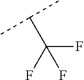 | O | H | H | H |
| 2-40 | 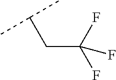 | O | H | H | H |
| 2-41 | 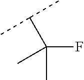 | O | H | H | H |
| 2-42 | 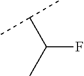 | O | H | H | H |
| 2-43 | 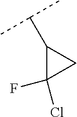 | O | H | H | H |
| 2-44 | 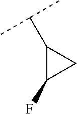 | O | H | H | H |
| 2-45 | 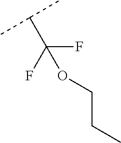 | O | H | H | H |
| 2-46 | 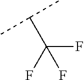 | O | H | 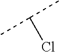 | H |
| 2-47 | 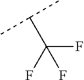 | O | H | 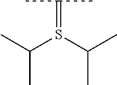 | |
| 2-48 | 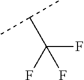 | O | H | 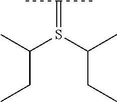 | |
| 2-49 | 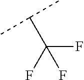 | O | H | 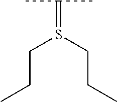 | |
| 2-50 | 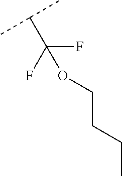 | O | H | H | H |
| 2-51 | 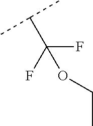 | O | H | H | H |
| 2-52 | 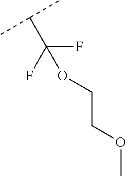 | O | H | H | H |
| 2-53 | 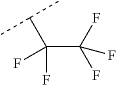 | O | 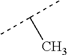 | H | H |
| 2-54 | 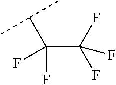 | O | 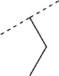 | H | H |
| 2-55 | 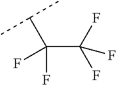 | O | 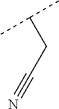 | H | H |
| 2-56 | 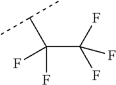 | O | 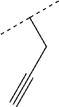 | H | H |
| 2-57 | 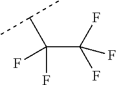 | O | 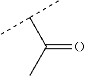 | H | H |
| 2-58 | 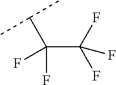 | O | 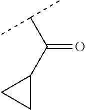 | H | H |
| 2-59 | 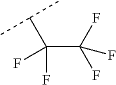 | O | 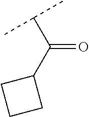 | H | H |
| 2-60 | 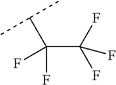 | O | 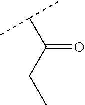 | H | H |
| 2-61 | 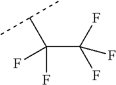 | O | 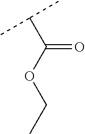 | H | H |
| 2-62 | 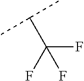 | O | 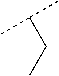 | H | H |
| 2-63 | 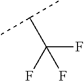 | O | 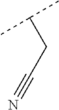 | H | H |
| 2-64 | 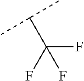 | O | 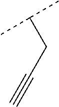 | H | H |
| 2-65 | 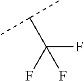 | O | 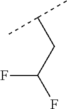 | H | H |
| 2-66 | 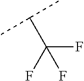 | O | 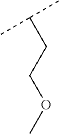 | H | H |
| 2-67 | 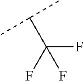 | O | H | 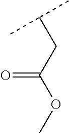 | H |
| 2-68 | 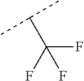 | O | H | 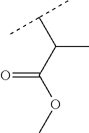 | H |
| 2-69 | 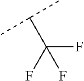 | O | 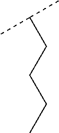 | H | H |
| 2-70 | 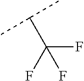 | O | 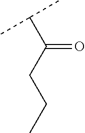 | H | H |
| 2-71 | 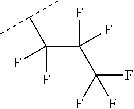 | O | 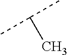 | H | H |
| 2-72 | 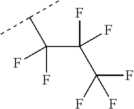 | O | 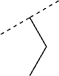 | H | H |
| 2-73 | 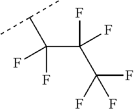 | O | 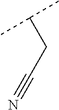 | H | H |
| 2-74 | 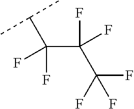 | O | 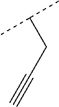 | H | H |
| 2-75 | 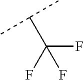 | O | 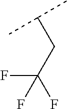 | H | H |
| 2-76 | 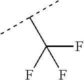 | O | 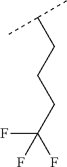 | H | H |
| 2-77 | 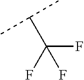 | O | 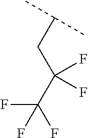 | H | H |
| 2-78 | 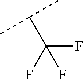 | O | 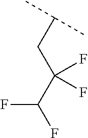 | H | H |
| 2-79 | 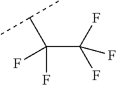 | O | 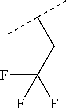 | H | H |
| 2-80 | 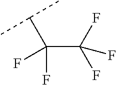 | O | 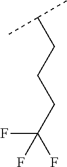 | H | H |
| 2-81 | 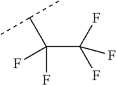 | O | 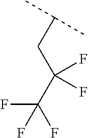 | H | H |
| 2-82 | 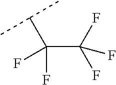 | O | 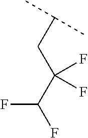 | H | H |
| 2-83 | 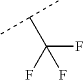 | O | H | 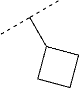 | H |
| 2-84 | 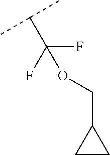 | O | H | H | H |
| 2-85 | 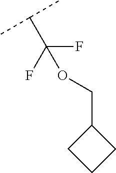 | O | H | H | H |
| 2-86 | 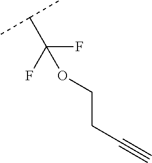 | O | H | H | H |
| 2-87 | 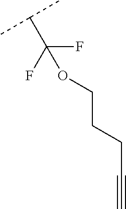 | O | H | H | H |
| 2-88 | 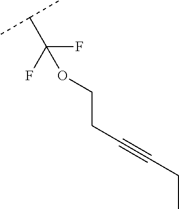 | O | H | H | H |
| 2-89 | 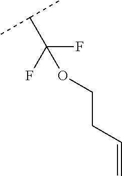 | O | H | H | H |
| 2-90 | 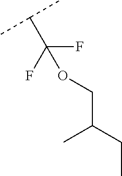 | O | H | H | H |
| 2-91 | 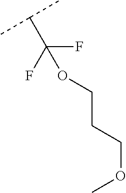 | O | H | H | H |
| 2-92 | 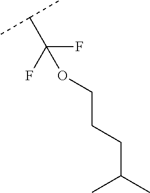 | O | H | H | H |
| 2-93 | 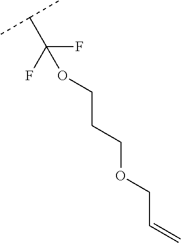 | O | H | H | H |
| 2-94 | 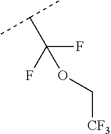 | O | H | H | H |
| 2-95 | 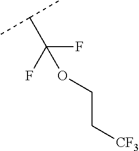 | O | H | H | H |
| 2-96 | 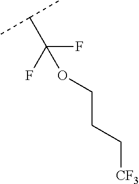 | O | H | H | H |
| 2-97 | 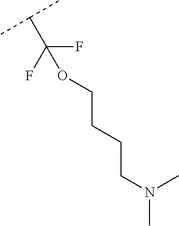 | O | H | H | H |
| 2-98 | 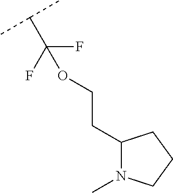 | O | H | H | H |
| 2-99 | 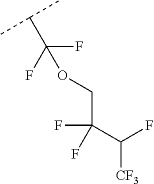 | O | H | H | H |
| 2-100 | 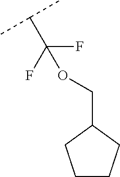 | O | H | H | H |
| 2-101 | 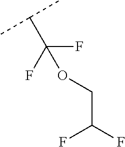 | O | H | H | H |
| 2-102 | 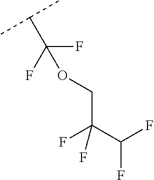 | O | H | H | H |
| 2-103 | 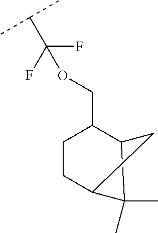 | O | H | H | H |

TABLE 3
(I)

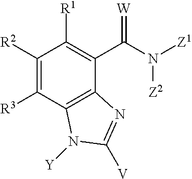

where R¹, R³ = H and R² = CF₃

| No. | V | W | Y | Z¹ | Z² |
|---|---|---|---|---|---|
| 3-1 | 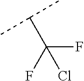 | O | H | H | H |
| 3-2 | 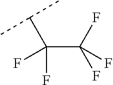 | O | H | H | H |
| 3-3 | 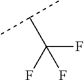 | O | H | 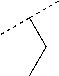 | H |
| 3-4 | 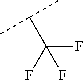 | O | 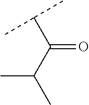 | H | H |
| 3-5 | 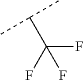 | O | H | 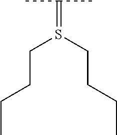 | |
| 3-6 | 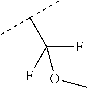 | O | H | H | H |
| 3-8 | 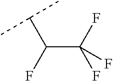 | O | H | H | H |
| 3-9 | 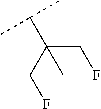 | O | H | H | H |
| 3-10 | 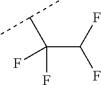 | O | H | H | H |
| 3-11 | 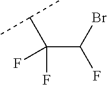 | O | H | H | H |
| 3-12 | 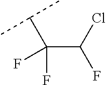 | O | H | H | H |
| 3-13 | 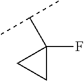 | O | H | H | H |
| 3-14 | 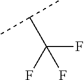 | O | H | H | H |
| 3-15 | 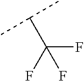 | O | H | 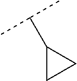 | H |
| 3-16 | 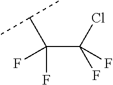 | O | H | H | H |
| 3-17 | 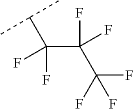 | O | H | H | H |
| 3-18 | 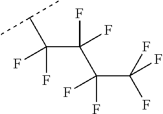 | O | H | H | H |
| 3-19 | 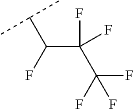 | O | H | H | H |
| 3-20 | 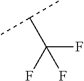 | O | H | 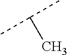 | H |
| 3-21 | 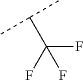 | O | H | 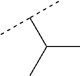 | H |
| 3-22 | 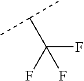 | O | H | 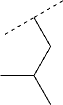 | H |
| 3-23 | 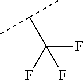 | O | H | 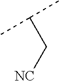 | H |
| 3-24 | 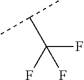 | O | H | 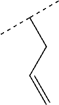 | H |
| 3-25 | 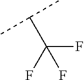 | O | H | 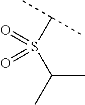 | H |
| 3-26 | 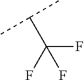 | O | H | 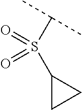 | H |
| 3-27 | 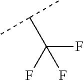 | O | 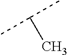 | H | H |
| 3-28 | 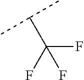 | O | 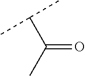 | H | H |
| 3-29 | 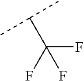 | O | 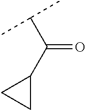 | H | H |
| 3-30 | 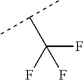 | O | 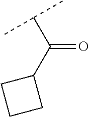 | H | H |
| 3-31 | 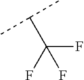 | O | 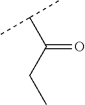 | H | H |
| 3-32 | 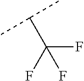 | O | 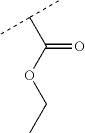 | H | H |
| 3-33 | 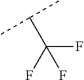 | O | H | 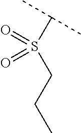 | H |
| 3-34 | 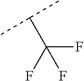 | O | H | 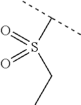 | H |
| 3-35 | 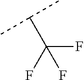 | O | H | 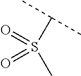 | H |
| 3-36 | 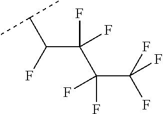 | O | H | H | H |
| 3-37 | 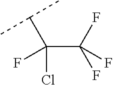 | O | H | H | H |
| 3-38 | 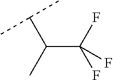 | O | H | H | H |
| 3-39 | 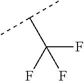 | S | H | H | H |
| 3-40 | 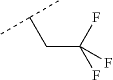 | O | H | H | H |
| 3-41 | 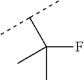 | O | H | H | H |
| 3-42 | 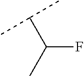 | O | H | H | H |
| 3-43 | 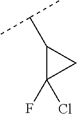 | O | H | H | H |
| 3-44 | 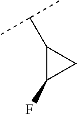 | O | H | H | H |
| 3-45 | 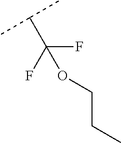 | O | H | H | H |
| 3-46 | 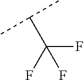 | O | H | 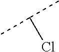 | H |
| 3-47 | 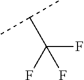 | O | H | 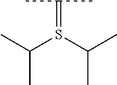 | |
| 3-48 | 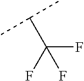 | O | H | 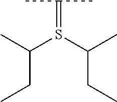 | |
| 3-49 | 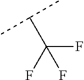 | O | H | 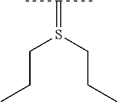 | |
| 3-50 | 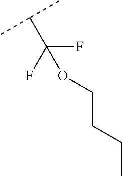 | O | H | H | H |
| 3-51 | 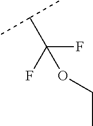 | O | H | H | H |
| 3-52 | 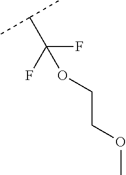 | O | H | H | H |
| 3-53 | 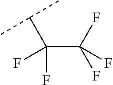 | O | 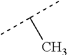 | H | H |
| 3-54 | 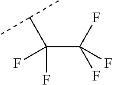 | O | 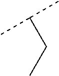 | H | H |
| 3-55 | 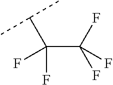 | O | 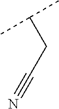 | H | H |
| 3-56 | 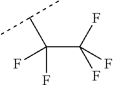 | O | 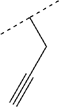 | H | H |
| 3-57 | 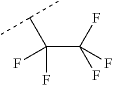 | O | 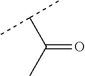 | H | H |
| 3-58 | 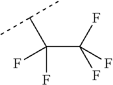 | O | 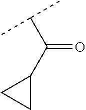 | H | H |
| 3-59 | 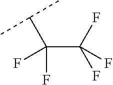 | O | 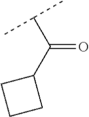 | H | H |
| 3-60 | 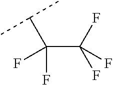 | O | 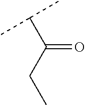 | H | H |
| 3-61 | 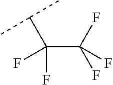 | O | 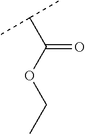 | H | H |
| 3-62 | 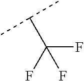 | O | 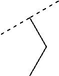 | H | H |
| 3-63 | 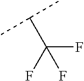 | O | 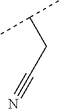 | H | H |
| 3-64 | 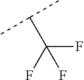 | O | 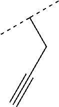 | H | H |
| 3-65 | 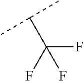 | O | 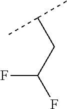 | H | H |
| 3-66 | 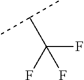 | O | 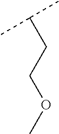 | H | H |
| 3-67 | 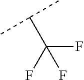 | O | H | 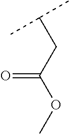 | H |
| 3-68 | 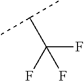 | O | H | 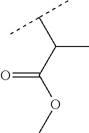 | H |
| 3-69 | 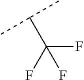 | O | 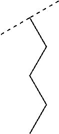 | H | H |
| 3-70 | 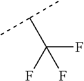 | O | 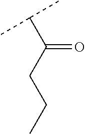 | H | H |
| 3-71 | 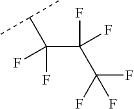 | O | 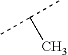 | H | H |
| 3-72 | 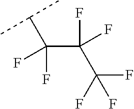 | O | 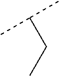 | H | H |
| 3-73 | 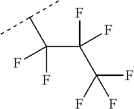 | O | 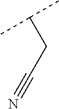 | H | H |
| 3-74 | 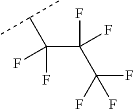 | O | 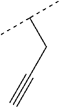 | H | H |
| 3-75 | 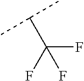 | O | 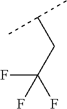 | H | H |
| 3-76 | 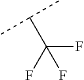 | O | 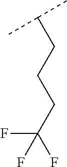 | H | H |
| 3-77 | 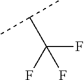 | O | 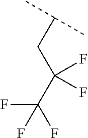 | H | H |
| 3-78 | 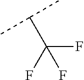 | O | 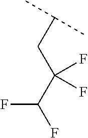 | H | H |
| 3-79 | 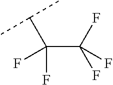 | O | 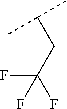 | H | H |
| 3-80 | 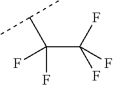 | O | 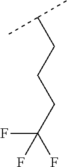 | H | H |
| 3-81 | 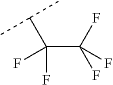 | O | 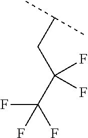 | H | H |
| 3-82 | 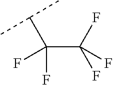 | O | 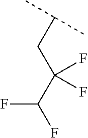 | H | H |
| 3-83 | 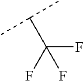 | O | H | 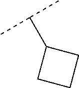 | H |
| 3-84 | 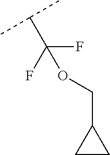 | O | H | H | H |
| 3-85 | 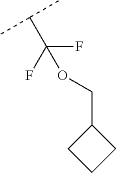 | O | H | H | H |
| 3-86 | 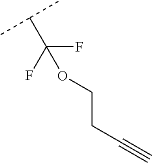 | O | H | H | H |
| 3-87 | 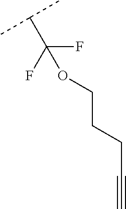 | O | H | H | H |
| 3-88 | 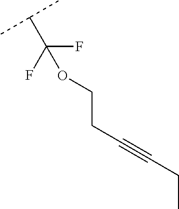 | O | H | H | H |
| 3-89 | 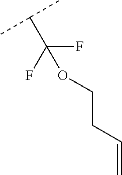 | O | H | H | H |
| 3-90 | 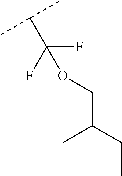 | O | H | H | H |
| 3-91 | 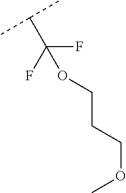 | O | H | H | H |
| 3-92 | 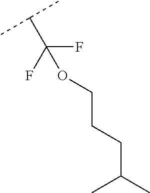 | O | H | H | H |
| 3-93 | 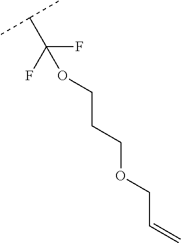 | O | H | H | H |
| 3-94 | 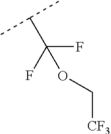 | O | H | H | H |
| 3-95 | 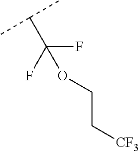 | O | H | H | H |
| 3-96 | 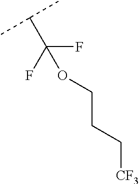 | O | H | H | H |
| 3-97 | 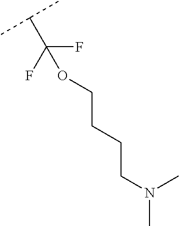 | O | H | H | H |
| 3-98 | 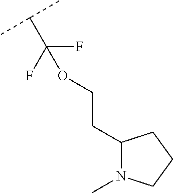 | O | H | H | H |
| 3-99 | 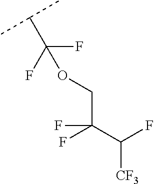 | O | H | H | H |
| 3-100 | 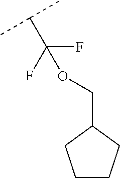 | O | H | H | H |
| 3-101 | 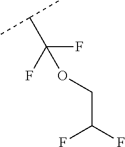 | O | H | H | H |
| 3-102 | 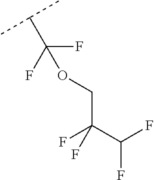 | O | H | H | H |
| 3-103 | 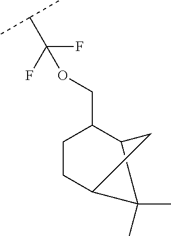 | O | H | H | H |

TABLE 4
(I)

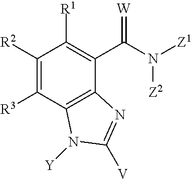

where R¹, R³ = H and R² = CH₃

| No. | V | W | Y | Z¹ | Z² |
|---|---|---|---|---|---|
| 4-1 | 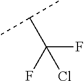 | O | H | H | H |
| 4-2 | 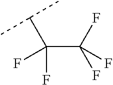 | O | H | H | H |
| 4-3 | 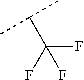 | O | H | 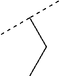 | H |
| 4-4 | 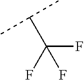 | O | 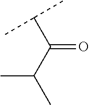 | H | H |
| 4-5 | 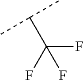 | O | H | 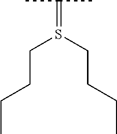 | |
| 4-6 | 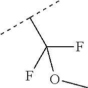 | O | H | H | H |
| 4-7 | 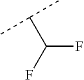 | O | H | H | H |
| 4-8 | 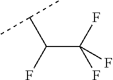 | O | H | H | H |
| 4-9 | 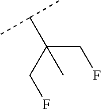 | O | H | H | H |
| 4-10 | 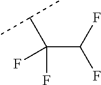 | O | H | H | H |
| 4-11 | 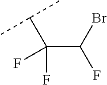 | O | H | H | H |
| 4-12 | 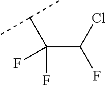 | O | H | H | H |
| 4-13 | 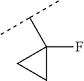 | O | H | H | H |
| 4-15 | 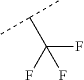 | O | H | 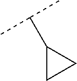 | H |
| 4-16 | 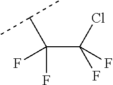 | O | H | H | H |
| 4-17 | 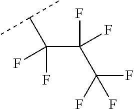 | O | H | H | H |
| 4-18 | 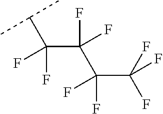 | O | H | H | H |
| 4-19 | 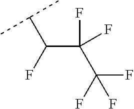 | O | H | H | H |
| 4-20 | 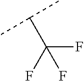 | O | H | 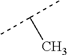 | H |
| 4-21 | 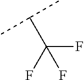 | O | H | 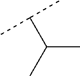 | H |
| 4-22 | 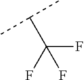 | O | H | 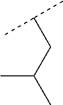 | H |
| 4-23 | 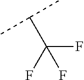 | O | H | 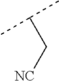 | H |
| 4-24 | 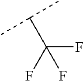 | O | H | 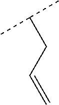 | H |
| 4-25 | 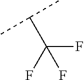 | O | H | 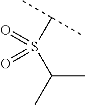 | H |
| 4-26 | 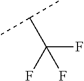 | O | H | 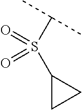 | H |
| 4-27 | 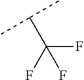 | O | 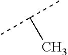 | H | H |
| 4-28 | 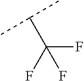 | O | 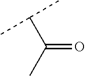 | H | H |
| 4-29 | 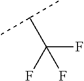 | O | 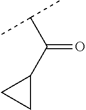 | H | H |
| 4-30 | 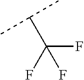 | O | 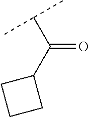 | H | H |
| 4-31 | 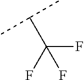 | O | 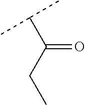 | H | H |
| 4-32 | 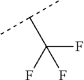 | O | 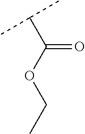 | H | H |
| 4-33 | 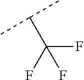 | O | H | 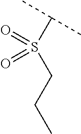 | H |
| 4-34 | 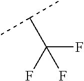 | O | H | 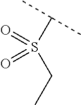 | H |
| 4-35 | 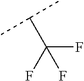 | O | H | 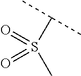 | H |
| 4-36 | 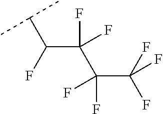 | O | H | H | H |
| 4-37 | 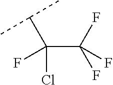 | O | H | H | H |
| 4-38 | 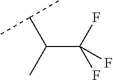 | O | H | H | H |
| 4-39 | 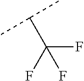 | S | H | H | H |
| 4-40 | 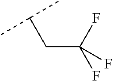 | O | H | H | H |
| 4-41 | 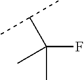 | O | H | H | H |
| 4-42 | 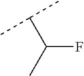 | O | H | H | H |
| 4-43 | 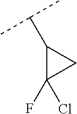 | O | H | H | H |
| 4-44 | 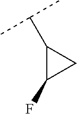 | O | H | H | H |
| 4-45 | 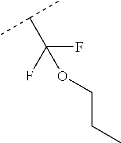 | O | H | H | H |
| 4-46 | 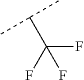 | O | H | 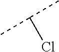 | H |
| 4-47 | 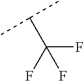 | O | H | 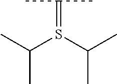 | |
| 4-48 | 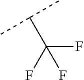 | O | H | 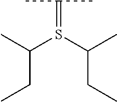 | |
| 4-49 | 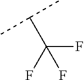 | O | H | 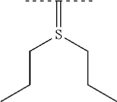 | |
| 4-50 | 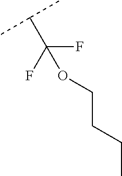 | O | H | H | H |
| 4-51 | 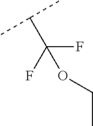 | O | H | H | H |
| 4-52 | 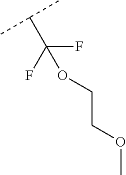 | O | H | H | H |
| 4-53 | 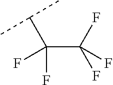 | O | 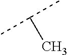 | H | H |
| 4-54 | 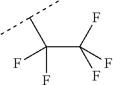 | O | 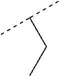 | H | H |
| 4-55 | 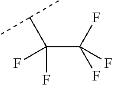 | O | 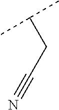 | H | H |
| 4-56 | 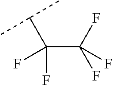 | O | 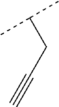 | H | H |
| 4-57 | 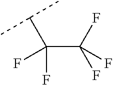 | O | 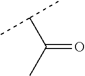 | H | H |
| 4-58 | 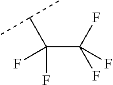 | O | 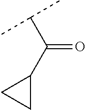 | H | H |
| 4-59 | 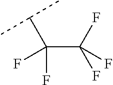 | O | 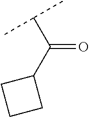 | H | H |
| 4-60 | 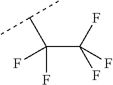 | O | 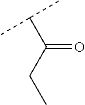 | H | H |
| 4-61 | 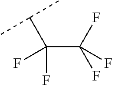 | O | 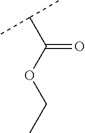 | H | H |
| 4-62 | 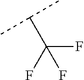 | O | 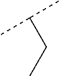 | H | H |
| 4-63 | 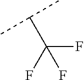 | O | 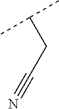 | H | H |
| 4-64 | 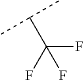 | O | 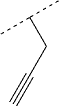 | H | H |
| 4-65 | 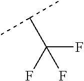 | O | 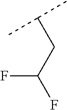 | H | H |
| 4-66 | 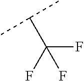 | O | 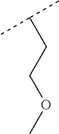 | H | H |
| 4-67 | 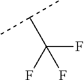 | O | H | 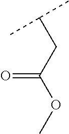 | H |
| 4-68 | 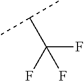 | O | H | 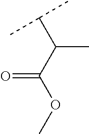 | H |
| 4-69 | 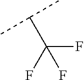 | O | 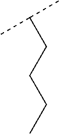 | H | H |
| 4-70 | 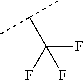 | O | 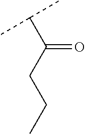 | H | H |
| 4-71 | 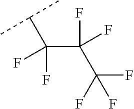 | O | 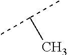 | H | H |
| 4-72 | 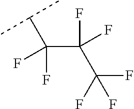 | O | 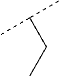 | H | H |
| 4-73 | 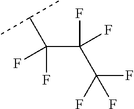 | O | 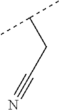 | H | H |
| 4-74 | 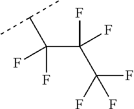 | O | 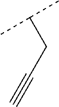 | H | H |
| 4-75 | 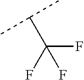 | O | 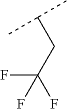 | H | H |
| 4-76 | 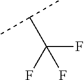 | O | 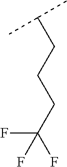 | H | H |
| 4-77 | 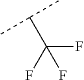 | O | 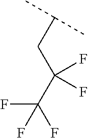 | H | H |
| 4-78 | 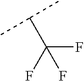 | O | 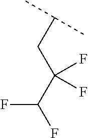 | H | H |
| 4-79 | 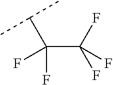 | O | 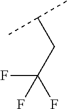 | H | H |
| 4-80 | 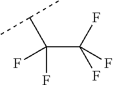 | O | 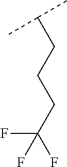 | H | H |
| 4-81 | 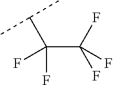 | O | 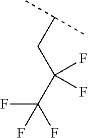 | H | H |
| 4-82 | 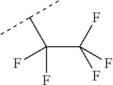 | O | 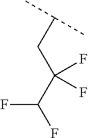 | H | H |
| 4-83 | 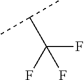 | O | H | 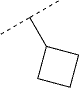 | H |
| 4-84 | 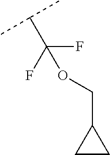 | O | H | H | H |
| 4-85 | 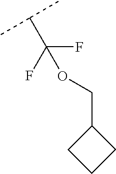 | O | H | H | H |
| 4-86 | 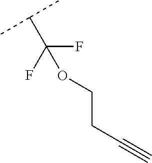 | O | H | H | H |
| 4-87 | 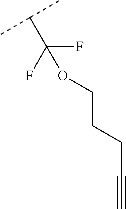 | O | H | H | H |
| 4-88 | 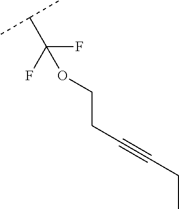 | O | H | H | H |
| 4-89 | 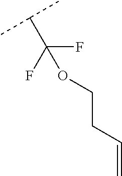 | O | H | H | H |
| 4-90 | 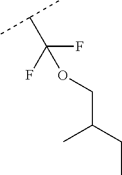 | O | H | H | H |
| 4-91 | 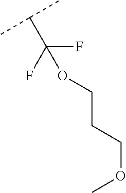 | O | H | H | H |
| 4-92 | 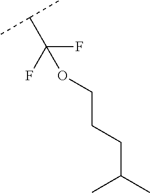 | O | H | H | H |
| 4-93 | 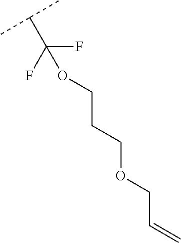 | O | H | H | H |
| 4-94 | 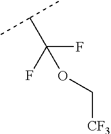 | O | H | H | H |
| 4-95 | 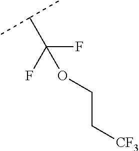 | O | H | H | H |
| 4-96 | 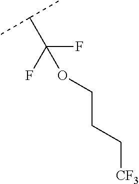 | O | H | H | H |
| 4-97 | 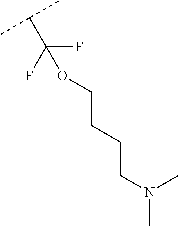 | O | H | H | H |
| 4-98 | 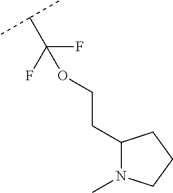 | O | H | H | H |
| 4-99 | 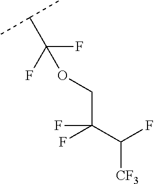 | O | H | H | H |
| 4-100 | 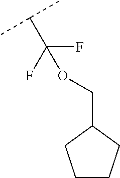 | O | H | H | H |
| 4-101 | 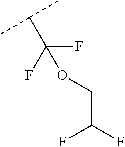 | O | H | H | H |
| 4-102 | 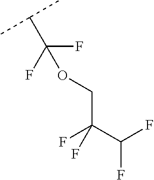 | O | H | H | H |
| 4-103 | 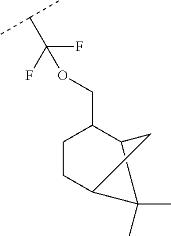 | O | H | H | H |

TABLE 5
(I)

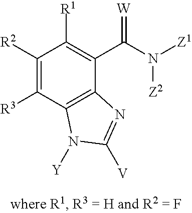

| No. | V | W | Y | Z¹ | Z² |
|---|---|---|---|---|---|
| 5-1 | 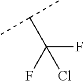 | O | H | H | H |
| 5-2 | 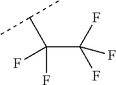 | O | H | H | H |
| 5-3 | 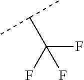 | O | H | 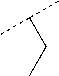 | H |
| 5-4 | 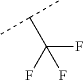 | O | 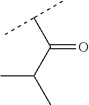 | H | H |
| 5-5 | 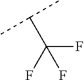 | O | H | 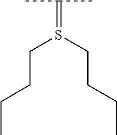 | |
| 5-6 | 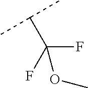 | O | H | H | H |
| 5-7 | 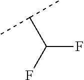 | O | H | H | H |
| 5-8 | 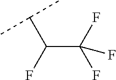 | O | H | H | H |
| 5-9 | 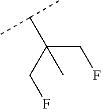 | O | H | H | H |
| 5-10 | 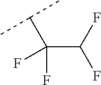 | O | H | H | H |
| 5-11 | 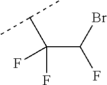 | O | H | H | H |
| 5-12 | 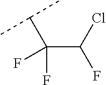 | O | H | H | H |
| 5-13 | 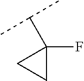 | O | H | H | H |
| 5-14 | 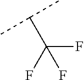 | O | H | H | H |
| 5-15 | 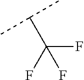 | O | H | 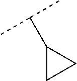 | H |
| 5-16 | 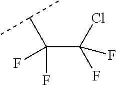 | O | H | H | H |
| 5-17 | 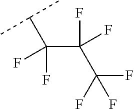 | O | H | H | H |
| 5-18 | 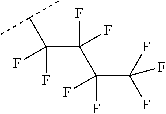 | O | H | H | H |
| 5-19 | 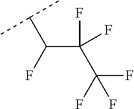 | O | H | H | H |
| 5-20 | 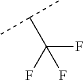 | O | H | 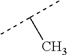 | H |
| 5-21 | 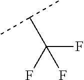 | O | H | 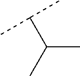 | H |
| 5-22 | 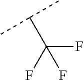 | O | H | 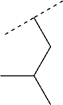 | H |
| 5-23 | 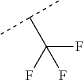 | O | H | 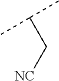 | H |
| 5-24 | 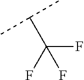 | O | H | 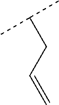 | H |
| 5-25 | 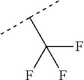 | O | H | 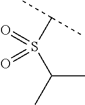 | H |
| 5-26 | 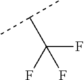 | O | H | 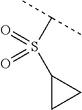 | H |
| 5-27 | 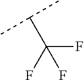 | O | 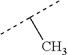 | H | H |
| 5-28 | 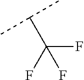 | O | 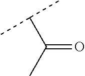 | H | H |
| 5-29 | 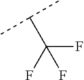 | O | 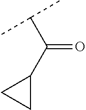 | H | H |
| 5-30 | 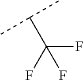 | O | 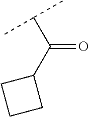 | H | H |
| 5-31 | 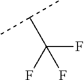 | O | 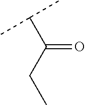 | H | H |
| 5-32 | 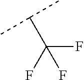 | O | 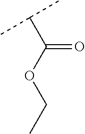 | H | H |
| 5-33 | 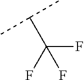 | O | H | 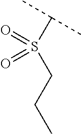 | H |
| 5-34 | 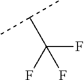 | O | H | 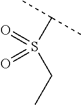 | H |
| 5-35 | 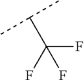 | O | H | 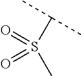 | H |
| 5-36 | 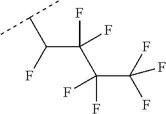 | O | H | H | H |
| 5-37 | 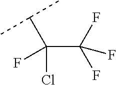 | O | H | H | H |
| 5-38 | 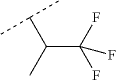 | O | H | H | H |
| 5-39 | 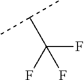 | S | H | H | H |
| 5-40 | 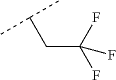 | O | H | H | H |
| 5-41 | 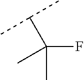 | O | H | H | H |
| 5-42 | 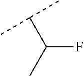 | O | H | H | H |
| 5-43 | 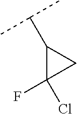 | O | H | H | H |
| 5-44 | 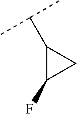 | O | H | H | H |
| 5-45 | 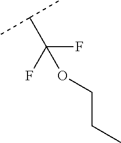 | O | H | H | H |
| 5-46 | 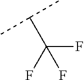 | O | H | 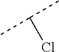 | H |
| 5-47 | 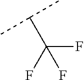 | O | H | 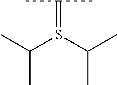 | |
| 5-48 | 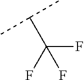 | O | H | 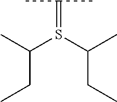 | |
| 5-49 | 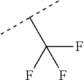 | O | H | 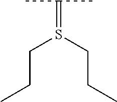 | |
| 5-50 | 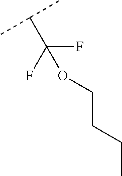 | O | H | H | H |
| 5-51 | 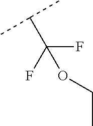 | O | H | H | H |
| 5-52 | 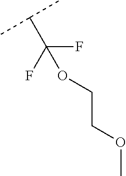 | O | H | H | H |
| 5-53 | 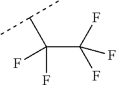 | O | 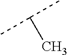 | H | H |
| 5-54 | 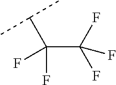 | O | 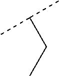 | H | H |
| 5-55 | 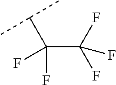 | O | 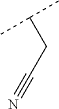 | H | H |
| 5-56 | 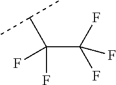 | O | 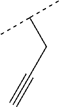 | H | H |
| 5-57 | 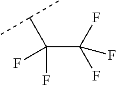 | O | 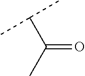 | H | H |
| 5-58 | 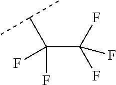 | O | 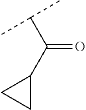 | H | H |
| 5-59 | 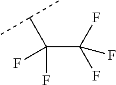 | O | 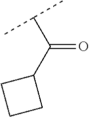 | H | H |
| 5-60 | 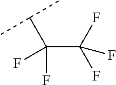 | O | 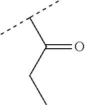 | H | H |
| 5-61 | 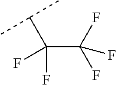 | O | 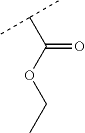 | H | H |
| 5-62 | 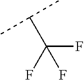 | O | 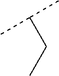 | H | H |
| 5-63 | 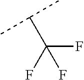 | O | 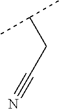 | H | H |
| 5-64 | 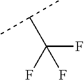 | O | 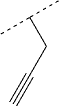 | H | H |
| 5-65 | 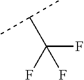 | O | 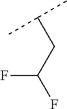 | H | H |
| 5-66 | 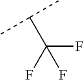 | O | 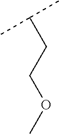 | H | H |
| 5-67 | 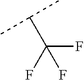 | O | H | 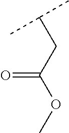 | H |
| 5-68 | 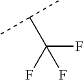 | O | H | 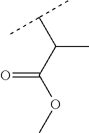 | H |
| 5-69 | 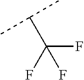 | O | 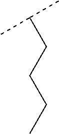 | H | H |
| 5-70 | 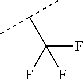 | O | 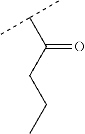 | H | H |
| 5-71 | 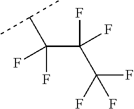 | O | 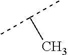 | H | H |
| 5-72 | 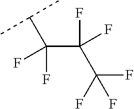 | O | 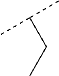 | H | H |
| 5-73 | 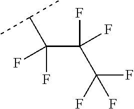 | O | 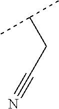 | H | H |
| 5-74 | 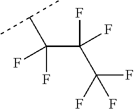 | O | 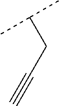 | H | H |
| 5-75 | 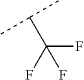 | O | 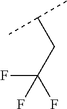 | H | H |
| 5-76 | 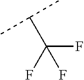 | O | 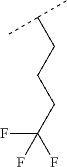 | H | H |
| 5-77 | 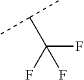 | O | 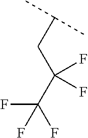 | H | H |
| 5-78 | 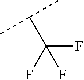 | O | 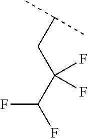 | H | H |
| 5-79 | 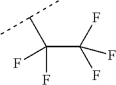 | O | 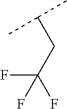 | H | H |
| 5-80 | 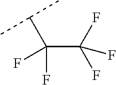 | O | 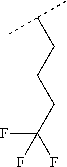 | H | H |
| 5-81 | 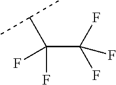 | O | 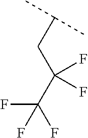 | H | H |
| 5-82 | 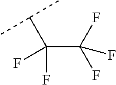 | O | 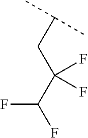 | H | H |
| 5-83 | 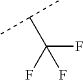 | O | H | 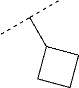 | H |
| 5-84 | 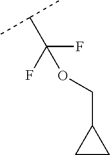 | O | H | H | H |
| 5-85 | 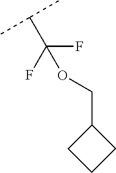 | O | H | H | H |
| 5-86 | 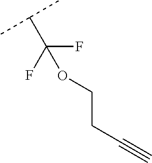 | O | H | H | H |
| 5-87 | 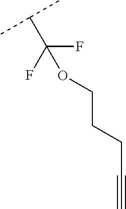 | O | H | H | H |
| 5-88 | 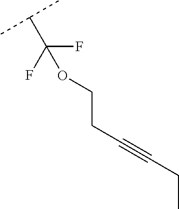 | O | H | H | H |
| 5-89 | 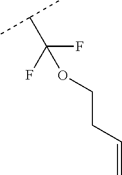 | O | H | H | H |
| 5-90 | 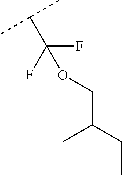 | O | H | H | H |
| 5-91 | 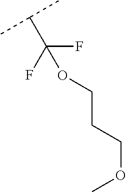 | O | H | H | H |
| 5-92 | 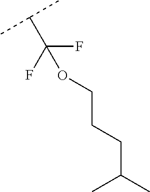 | O | H | H | H |
| 5-93 | 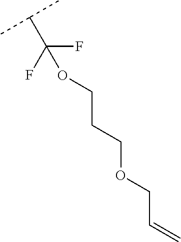 | O | H | H | H |
| 5-94 | 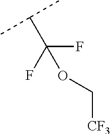 | O | H | H | H |
| 5-95 | 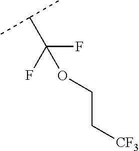 | O | H | H | H |
| 5-96 | 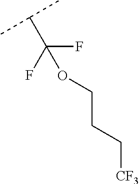 | O | H | H | H |
| 5-97 | 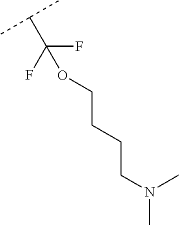 | O | H | H | H |
| 5-98 | 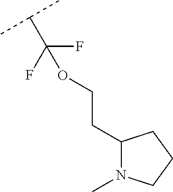 | O | H | H | H |
| 5-99 | 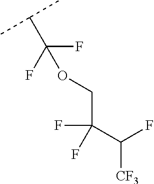 | O | H | H | H |
| 5-100 | 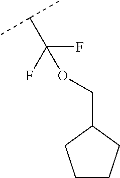 | O | H | H | H |
| 5-101 | 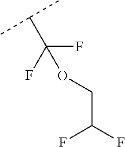 | O | H | H | H |
| 5-102 | 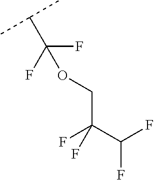 | O | H | H | H |
| 5-103 | 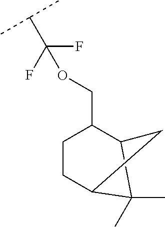 | O | H | H | H |

TABLE 6
(I)

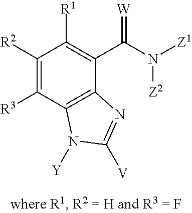

| No. | V | W | Y | Z¹ | Z² |
|---|---|---|---|---|---|
| 6-1 | 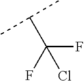 | O | H | H | H |
| 6-2 | 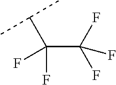 | O | H | H | H |
| 6-3 | 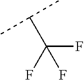 | O | H | 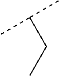 | H |
| 6-4 | 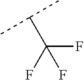 | O | 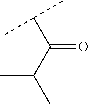 | H | H |
| 6-5 | 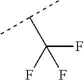 | O | H | 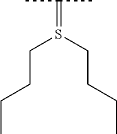 | |
| 6-6 | 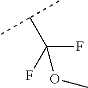 | O | H | H | H |
| 6-7 | 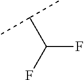 | O | H | H | H |
| 6-8 | 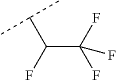 | O | H | H | H |
| 6-9 | 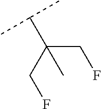 | O | H | H | H |
| 6-10 | 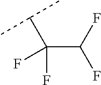 | O | H | H | H |
| 6-11 | 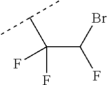 | O | H | H | H |
| 6-12 | 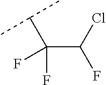 | O | H | H | H |
| 6-13 | 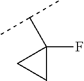 | O | H | H | H |
| 6-14 | 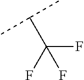 | O | H | H | H |
| 6-15 | 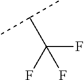 | O | H | 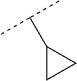 | H |
| 6-16 | 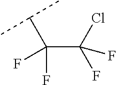 | O | H | H | H |
| 6-17 | 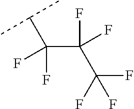 | O | H | H | H |
| 6-18 | 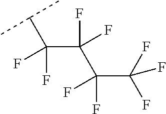 | O | H | H | H |
| 6-19 | 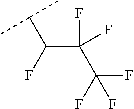 | O | H | H | H |
| 6-20 | 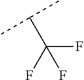 | O | H | 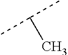 | H |
| 6-21 | 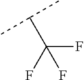 | O | H | 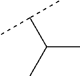 | H |
| 6-22 | 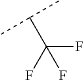 | O | H | 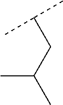 | H |
| 6-23 | 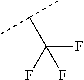 | O | H | 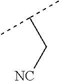 | H |
| 6-24 | 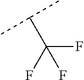 | O | H | 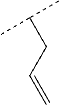 | H |
| 6-25 | 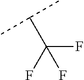 | O | H | 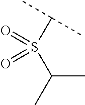 | H |
| 6-26 | 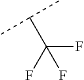 | O | H | 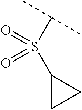 | H |
| 6-27 | 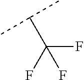 | O | 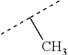 | H | H |
| 6-28 | 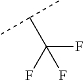 | O | 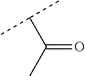 | H | H |
| 6-29 | 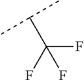 | O | 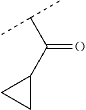 | H | H |
| 6-30 | 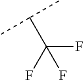 | O | 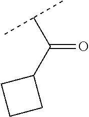 | H | H |
| 6-31 | 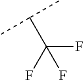 | O | 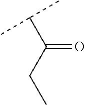 | H | H |
| 6-32 | 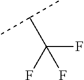 | O | 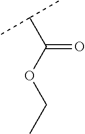 | H | H |
| 6-33 | 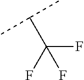 | O | H | 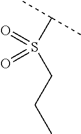 | H |
| 6-34 | 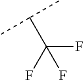 | O | H | 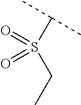 | H |
| 6-35 | 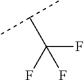 | O | H | 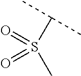 | H |
| 6-36 | 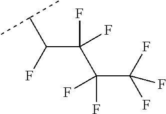 | O | H | H | H |
| 6-37 | 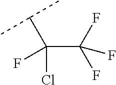 | O | H | H | H |
| 6-38 | 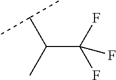 | O | H | H | H |
| 6-39 | 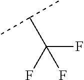 | S | H | H | H |
| 6-40 | 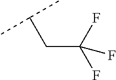 | O | H | H | H |
| 6-41 | 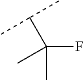 | O | H | H | H |
| 6-42 | 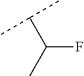 | O | H | H | H |
| 6-43 | 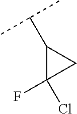 | O | H | H | H |
| 6-44 | 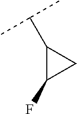 | O | H | H | H |
| 6-45 | 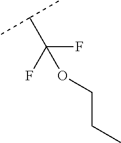 | O | H | H | H |
| 6-46 | 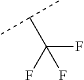 | O | H | 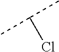 | H |
| 6-47 | 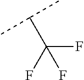 | O | H | 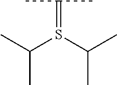 | |
| 6-48 | 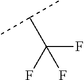 | O | H | 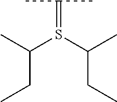 | |
| 6-49 | 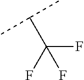 | O | H | 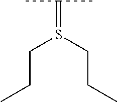 | |
| 6-50 | 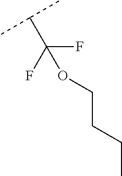 | O | H | H | H |
| 6-51 | 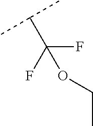 | O | H | H | H |
| 6-52 | 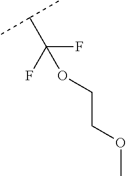 | O | H | H | H |
| 6-53 | 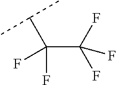 | O | 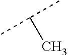 | H | H |
| 6-54 | 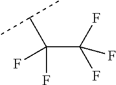 | O | 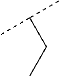 | H | H |
| 6-55 | 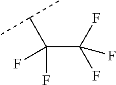 | O | 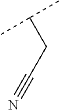 | H | H |
| 6-56 | 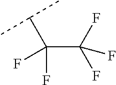 | O | 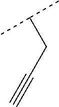 | H | H |
| 6-57 | 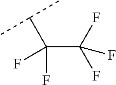 | O | 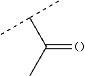 | H | H |
| 6-58 | 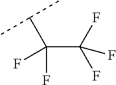 | O | 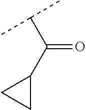 | H | H |
| 6-59 | 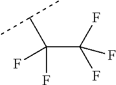 | O | 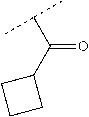 | H | H |
| 6-60 | 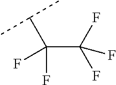 | O | 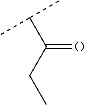 | H | H |
| 6-61 | 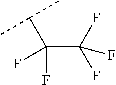 | O | 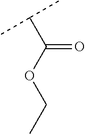 | H | H |
| 6-62 | 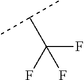 | O | 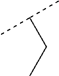 | H | H |
| 6-63 | 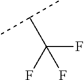 | O | 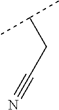 | H | H |
| 6-64 | 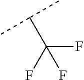 | O | 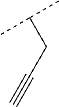 | H | H |
| 6-65 | 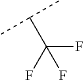 | O | 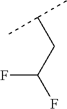 | H | H |
| 6-66 | 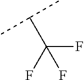 | O | 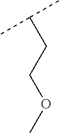 | H | H |
| 6-67 | 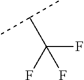 | O | H | 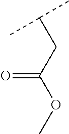 | H |
| 6-68 | 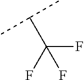 | O | H | 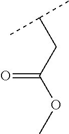 | H |
| 6-69 | 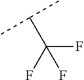 | O | 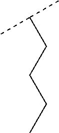 | H | H |
| 6-70 | 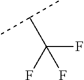 | O | 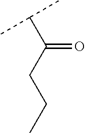 | H | H |
| 6-71 | 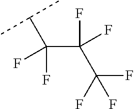 | O | 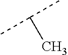 | H | H |
| 6-72 | 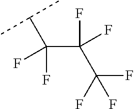 | O | 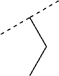 | H | H |
| 6-73 | 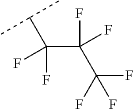 | O | 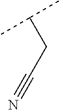 | H | H |
| 6-74 | 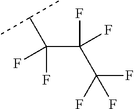 | O | 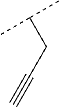 | H | H |
| 6-75 | 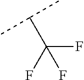 | O | 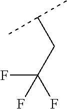 | H | H |
| 6-76 | 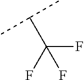 | O | 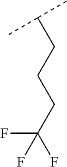 | H | H |
| 6-77 | 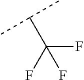 | O | 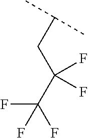 | H | H |
| 6-78 | 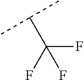 | O | 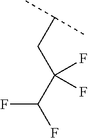 | H | H |
| 6-79 | 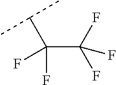 | O | 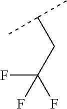 | H | H |
| 6-80 | 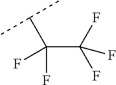 | O | 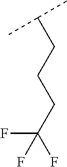 | H | H |
| 6-81 | 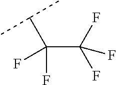 | O | 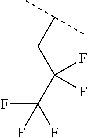 | H | H |
| 6-82 | 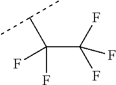 | O | 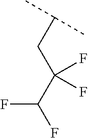 | H | H |
| 6-83 | 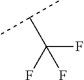 | O | H | 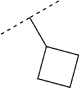 | H |
| 6-84 | 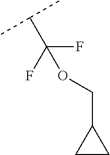 | O | H | H | H |
| 6-85 | 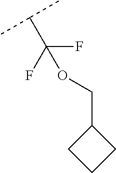 | O | H | H | H |
| 6-86 | 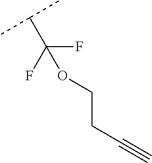 | O | H | H | H |
| 6-87 | 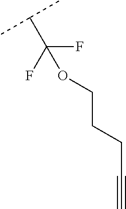 | O | H | H | H |
| 6-88 | 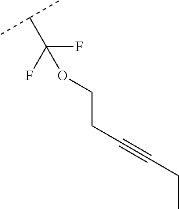 | O | H | H | H |
| 6-89 | 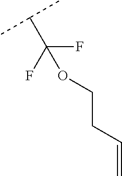 | O | H | H | H |
| 6-90 | 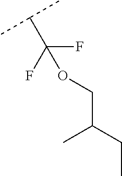 | O | H | H | H |
| 6-91 | 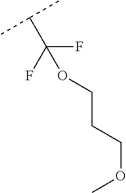 | O | H | H | H |
| 6-92 | 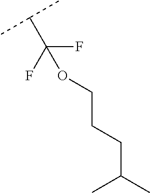 | O | H | H | H |
| 6-93 | 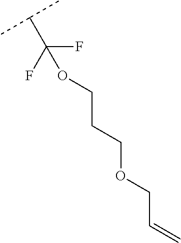 | O | H | H | H |
| 6-94 | 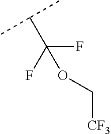 | O | H | H | H |
| 6-95 | 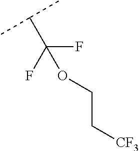 | O | H | H | H |
| 6-96 | 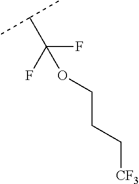 | O | H | H | H |
| 6-97 | 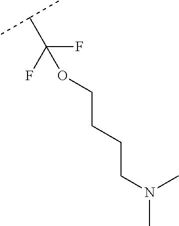 | O | H | H | H |
| 6-98 | 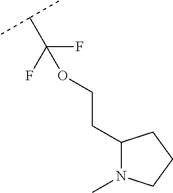 | O | H | H | H |
| 6-99 | 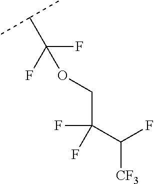 | O | H | H | H |
| 6-100 | 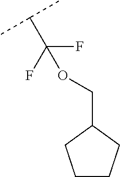 | O | H | H | H |
| 6-101 | 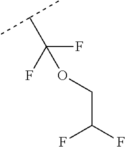 | O | H | H | H |
| 6-102 | 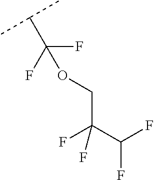 | O | H | H | H |
| 6-103 | 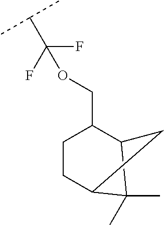 | O | H | H | H |

TABLE 7
(I)

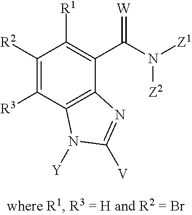

| No. | V | W | Y | Z¹ | Z² |
|---|---|---|---|---|---|
| 7-1 | 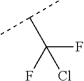 | O | H | H | H |
| 7-2 | 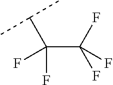 | O | H | H | H |
| 7-3 | 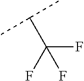 | O | H | 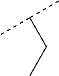 | H |
| 7-4 | 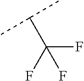 | O | 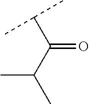 | H | H |
| 7-5 | 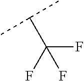 | O | H | 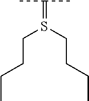 | |
| 7-6 | 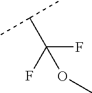 | O | H | H | H |
| 7-7 | 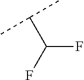 | O | H | H | H |
| 7-8 | 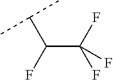 | O | H | H | H |
| 7-9 | 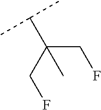 | O | H | H | H |
| 7-10 | 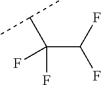 | O | H | H | H |
| 7-11 | 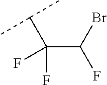 | O | H | H | H |
| 7-12 | 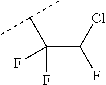 | O | H | H | H |
| 7-13 | 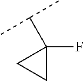 | O | H | H | H |
| 7-14 | 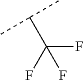 | O | H | H | H |
| 7-15 | 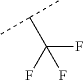 | O | H | 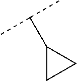 | H |
| 7-16 | 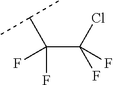 | O | H | H | H |
| 7-17 | 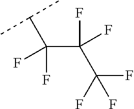 | O | H | H | H |
| 7-18 | 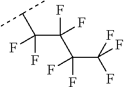 | O | H | H | H |
| 7-19 | 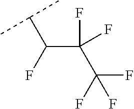 | O | H | H | H |
| 7-20 | 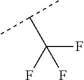 | O | H | 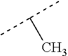 | H |
| 7-21 | 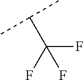 | O | H | 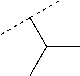 | H |
| 7-22 | 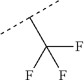 | O | H | 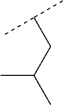 | H |
| 7-23 | 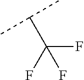 | O | H | 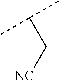 | H |
| 7-24 | 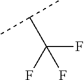 | O | H | 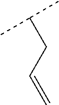 | H |
| 7-25 | 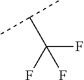 | O | H | 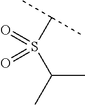 | H |
| 7-26 | 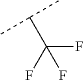 | O | H | 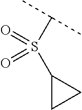 | H |
| 7-27 | 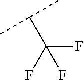 | O | 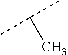 | H | H |
| 7-28 | 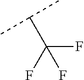 | O | 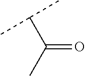 | H | H |
| 7-29 | 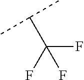 | O | 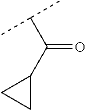 | H | H |
| 7-30 | 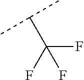 | O | 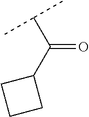 | H | H |
| 7-31 | 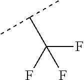 | O | 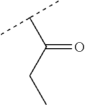 | H | H |
| 7-32 | 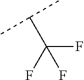 | O | 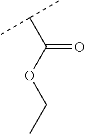 | H | H |
| 7-33 | 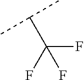 | O | H | 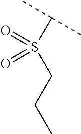 | H |
| 7-34 | 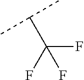 | O | H | 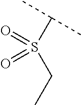 | H |
| 7-35 | 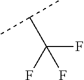 | O | H | 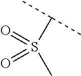 | H |
| 7-36 | 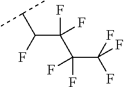 | O | H | H | H |
| 7-37 | 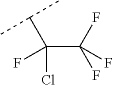 | O | H | H | H |
| 7-38 | 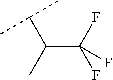 | O | H | H | H |
| 7-39 | 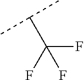 | S | H | H | H |
| 7-40 | 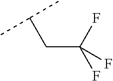 | O | H | H | H |
| 7-41 | 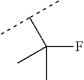 | O | H | H | H |
| 7-42 | 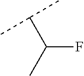 | O | H | H | H |
| 7-43 | 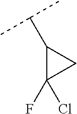 | O | H | H | H |
| 7-44 | 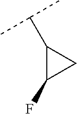 | O | H | H | H |
| 7-45 | 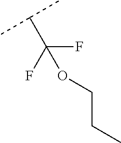 | O | H | H | H |
| 7-46 | 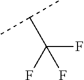 | O | H | 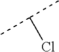 | H |
| 7-47 | 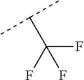 | O | H | 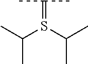 | |
| 7-48 | 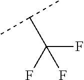 | O | H | 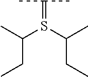 | |
| 7-49 | 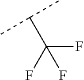 | O | H | 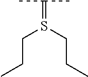 | |
| 7-50 | 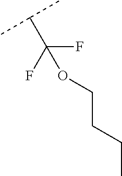 | O | H | H | H |
| 7-51 | 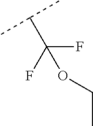 | O | H | H | H |
| 7-52 | 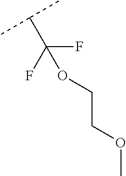 | O | H | H | H |
| 7-53 | 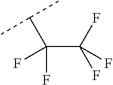 | O | 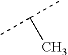 | H | H |
| 7-54 | 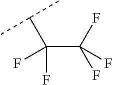 | O | 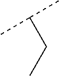 | H | H |
| 7-55 | 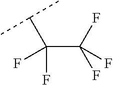 | O | 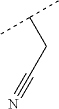 | H | H |
| 7-56 | 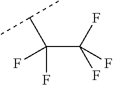 | O | 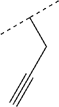 | H | H |
| 7-57 | 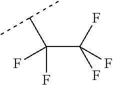 | O | 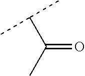 | H | H |
| 7-58 | 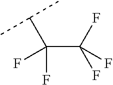 | O | 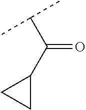 | H | H |
| 7-59 | 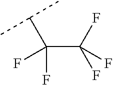 | O | 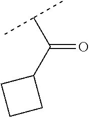 | H | H |
| 7-60 | 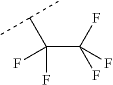 | O | 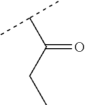 | H | H |
| 7-61 | 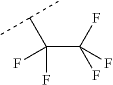 | O | 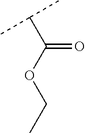 | H | H |
| 7-62 | 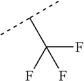 | O | 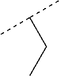 | H | H |
| 7-63 | 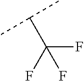 | O | 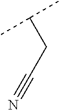 | H | H |
| 7-64 | 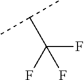 | O | 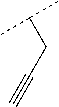 | H | H |
| 7-65 | 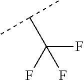 | O | 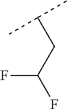 | H | H |
| 7-66 | 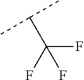 | O | 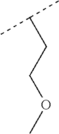 | H | H |
| 7-67 | 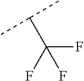 | O | H | 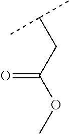 | H |
| 7-68 | 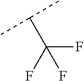 | O | H | 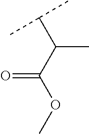 | H |
| 7-69 | 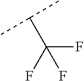 | O | 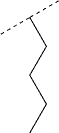 | H | H |
| 7-70 | 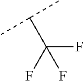 | O | 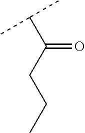 | H | H |
| 7-71 | 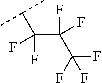 | O | 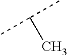 | H | H |
| 7-72 | 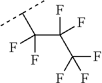 | O | 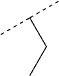 | H | H |
| 7-73 | 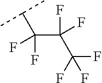 | O | 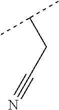 | H | H |
| 7-74 | 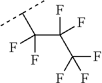 | O | 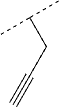 | H | H |
| 7-75 | 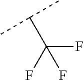 | O | 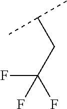 | H | H |
| 7-76 | 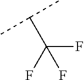 | O | 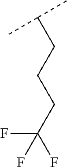 | H | H |
| 7-77 | 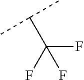 | O | 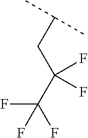 | H | H |
| 7-78 | 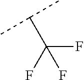 | O | 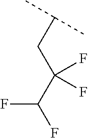 | H | H |
| 7-79 | 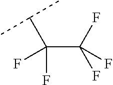 | O | 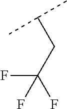 | H | H |
| 7-80 | 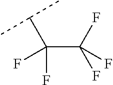 | O | 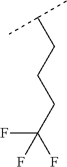 | H | H |
| 7-81 | 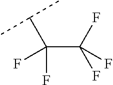 | O | 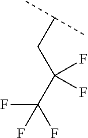 | H | H |
| 7-82 | 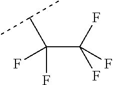 | O | 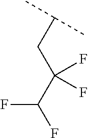 | H | H |
| 7-83 | 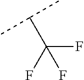 | O | H | 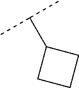 | H |
| 7-84 | 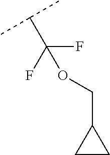 | O | H | H | H |
| 7-85 | 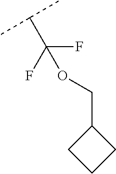 | O | H | H | H |
| 7-86 | 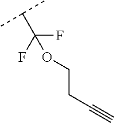 | O | H | H | H |
| 7-87 | 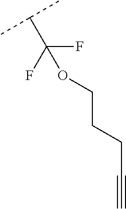 | O | H | H | H |
| 7-88 | 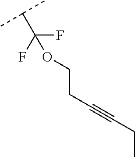 | O | H | H | H |
| 7-89 | 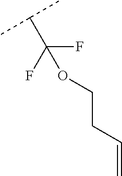 | O | H | H | H |
| 7-90 | 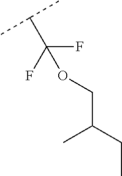 | O | H | H | H |
| 7-91 | 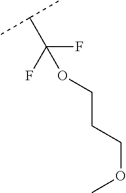 | O | H | H | H |
| 7-92 | 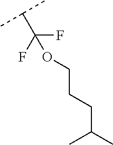 | O | H | H | H |
| 7-93 | 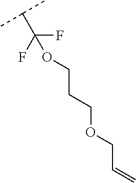 | O | H | H | H |
| 7-94 | 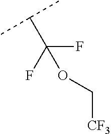 | O | H | H | H |
| 7-95 | 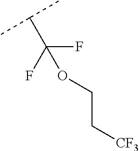 | O | H | H | H |
| 7-96 | 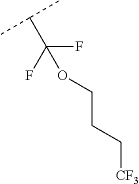 | O | H | H | H |
| 7-97 | 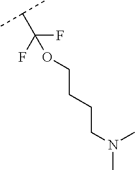 | O | H | H | H |
| 7-98 | 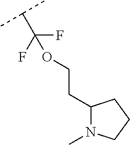 | O | H | H | H |
| 7-99 | 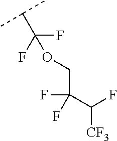 | O | H | H | H |
| 7-100 | 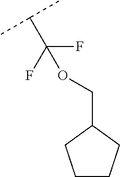 | O | H | H | H |
| 7-101 | 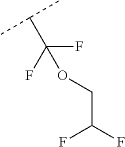 | O | H | H | H |
| 7-102 | 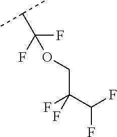 | O | H | H | H |
| 7-103 | 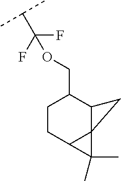 | O | H | H | H |

Spectroscopic data of the chemical examples:

### Example No. 1-7

¹H NMR (400 MHz, CDCl₃ δ, ppm) 13.68 (br. s, 1H, NH), 9.06 (br. s, 1H, NH), 8.01 (d, 1H), 7.80 (d, 1H), 7.46 (dd, 1H), 7.22-6.96 (t, 1H), 6.10 (br. s, 1H, NH).

### Example No. 1-8

¹H NMR (400 MHz, CDCl₃ δ, ppm) 13.24 (br. s, 1H, NH), 8.92 (br. s, 1H, NH), 8.08 (d, 1H), 7.78 (d, 1H), 7.45 (dd, 1H), 6.60-6.42 (dq, 1 H), 6.40 (br. s, 1H, NH).

### Example No. 1-9

¹H NMR (400 MHz, CDCl₃ δ, ppm) 9.82 (br. s, 1H, NH), 9.53 (br. s, 1H, NH), 8.14 (d, 1H), 7.62 (d, 1H), 7.37 (dd, 1H), 5.96 (br. s, 1H, NH), 4.87 (dd, 2H), 4.72 (dd, 2H), 1.62 (s, 3H).

### Example No. 1-10

¹H NMR (400 MHz, d₆-DMSO δ, ppm) 14.31 (br. s, 1H, NH), 8.79 (br. s, 1H, NH), 8.02 (d, 1H), 7.93 (br. s, 1H, NH), 7.84 (d, 1H), 7.53 (dd, 1H), 7.39-7.09 (tt, 1H).

### Example No. 1-11

¹H NMR (400 MHz, d₆-DMSO δ, ppm) 14.28 (br. s, 1H, NH), 8.81 (br. s, 1H, NH), 8.00 (d, 1H), 7.87 (br. s, 1H, NH), 7.82 (d, 1H), 7.52 (dd, 1H), 7.80-7.12 (dt, 1H).

### Example No. 1-12

¹H NMR (400 MHz, d₆-DMSO δ, ppm) 14.23 (br. s, 1H, NH), 8.79 (br. s, 1H, NH), 7.97 (d, 1H), 7.88 (br. s, 1H, NH), 7.81 (m, 1H), 7.65 (m, 1H), 7.52 (dd, 1H).

### Example No. 1-13

¹H NMR (400 MHz, CDCl₃ δ, ppm) 9.86 (br. s, 1H, NH), 9.28 (br. s, 1H, NH), 8.12 (d, 1H), 7.61 (d, 1H), 7.32 (dd, 1H), 5.81 (br. s, 1H, NH), 1.64 (m, 2H), 1.55 (m, 2H).

### Example No. 1-14

¹H NMR (400 MHz, d₆-DMSO δ, ppm) 14.16 (br. s, 1H, NH), 8.76 (br. s, 1H, NH), 7.98 (d, 1H), 7.91 (br. s, 1H, NH), 7.82 (d, 1H), 7.50 (dd, 1H).

### Example No. 1-15

¹H NMR (400 MHz, d₆-DMSO δ, ppm) 14.58 (br. s, 1H, NH), 9.23 (br. s, 1H, NH), 7.98 (m, 1H), 7.83 (m, 1H), 7.54 (m, 1H), 2.94 (m, 1H), 0.78 (m, 2H), 0.58 (m, 2H).

### Example No. 1-16

¹H NMR (400 MHz, d₆-DMSO δ, ppm) 14.15 (br. s, 1H, NH), 9.12 (br. s, 1H, NH), 7.83 (d, 1H), 7.77 (d, 1H), 7.60 (br. s, 1H, NH), 7.29 (dd, 1H); ¹⁹F NMR (375 MHz, CD₃OD δ, ppm) −126.3, −112.6, −80.1.

### Example No. 1-17

¹H NMR (400 MHz, d₆-DMSO δ, ppm) 14.62 (br. s, 1H, NH), 8.64 (br. s, 1H, NH), 8.00 (d, 1H), 7.88 (d, 1H), 7.72 (br. s, 1H, NH), 7.52 (dd, 1H).

### Example No. 1-18

¹H NMR (400 MHz, d₆-DMSO δ, ppm) 14.53 (br. s, 1H, NH), 8.60 (br. s, 1H, NH), 7.94 (d, 1H), 7.86 (d, 1H), 7.65 (br. s, 1H, NH), 7.49 (dd, 1H).

### Example No. 1-19

¹H NMR (400 MHz, d₆-DMSO δ, ppm) 14.32 (br. s, 1H, NH), 8.80 (br. s, 1H, NH), 8.00 (d, 1H), 7.93 (br. s, 1H, NH), 7.84 (d, 1H), 7.54 (dd, 1H), 7.03-6.85 (double sext, 1H).

### Example No. 1-20

¹H NMR (400 MHz, CDCl₃ δ, ppm) 9.35 (br. s, 1H, NH), 8.01 (d, 1H), 7.58 (d, 1H), 7.44 (dd, 1H), 6.41 (br. s, 1H, NH), 3.62 (s, 3H).

### Example No. 1-21

¹H NMR (400 MHz, CDCl₃ δ, ppm) 9.38 (br. s, 1H, NH), 8.04 (d, 1H), 7.55 (d, 1H), 7.38 (dd, 1H), 6.24 (br. s, 1H, NH), 4.32 (sept, 1H), 1.36 (d, 6H).

### Example No. 1-22

¹H NMR (400 MHz, CDCl₃ δ, ppm) 9.71 (br. s, 1H, NH), 8.24 (d, 1H), 7.72 (d, 1H), 7.49 (dd, 1H), 6.53 (br. s, 1H, NH), 3.48 (d, 1H), 3.45 (d, 1H), 2.01 (sept, 1H), 1.06 (d, 6H).

### Example No. 1-23

¹H NMR (400 MHz, CDCl₃ δ, ppm) 9.69 (br. s, 1H, NH), 8.31 (d, 1H), 7.74 (m, 1H), 7.59 (m, 1H), 6.48 (br. s, 1H, NH), 4.52 (d, 1H), 4.44 (d, 1H).

### Example No. 1-24

¹H NMR (400 MHz, CDCl₃ δ, ppm) 9.52 (br. s, 1H, NH), 8.29 (d, 1H), 7.71 (d, 1H), 7.41 (dd, 1H), 6.51 (br. s, 1H, NH), 5.98 (m, 1 H), 5.24 (m, 2H), 4.26 (d, 1H), 4.23 (d, 1H).

### Example No. 1-25

¹H NMR (400 MHz, d₆-DMSO δ, ppm) 8.02 (d, 1H), 7.86 (d, 1H), 7.69 (br. s, 1H, NH), 7.49 (dd, 1H), 6.62 (br. s, 1H, NH), 3.83 (sept, 1 H), 1.36 (d, 6H).

### Example No. 1-26

¹H NMR (400 MHz, d₆-DMSO δ, ppm) 8.03 (d, 1H), 7.97 (d, 1H), 7.58 (br. s, 1H, NH), 7.44 (dd, 1H), 6.78 (br. s, 1H, NH), 3.06 (m, 1H), 1.27 (m, 2H), 1.08 (m, 2H).

### Example No. 1-27

¹H NMR (400 MHz, d₆-DMSO δ, ppm) 8.62 (br. s, 1H, NH), 8.06 (d, 1H), 8.02 (d, 1H), 7.92 (br. s, 1H, NH), 7.61 (dd, 1H), 4.04 (s, 3H).

### Example No. 1-28

¹H NMR (400 MHz, d₆-DMSO δ, ppm) 9.34 (br. s, 1H, NH), 8.15 (d, 1H), 8.09 (br. s, 1H, NH), 8.01 (d, 1H), 7.62 (dd, 1H), 2.42 (s, 3H).

### Example No. 1-29

¹H NMR (400 MHz, CDCl₃ δ, ppm) 9.43 (br. s, 1H, NH), 8.35 (d, 1H), 8.04 (d, 1H), 7.64 (dd, 1H), 6.10 (br. s, 1H, NH), 2.39 (m, 1H), 1.64 (m, 2H), 1.43 (m, 2H).

### Example No. 1-30

¹H NMR (400 MHz, CDCl₃ δ, ppm) 9.30 (br. s, 1H, NH), 8.08 (d, 1H), 7.72 (d, 1H), 7.49 (dd, 1H), 6.24 (br. s, 1H, NH), 3.61 (quint, 1H), 2.25 (m, 4H), 1.97 (m, 1H), 1.70 (m, 1H).

### Example No. 1-31

¹H NMR (400 MHz, CDCl₃ δ, ppm) 9.41 (br. s, 1H, NH), 8.19 (d, 1H), 7.93 (d, 1H), 7.58 (dd, 1H), 6.08 (br. s, 1H, NH), 2.87 (q, 2H), 1.26 (t, 3H).

### Example No. 1-32

¹H NMR (400 MHz, CDCl₃ δ, ppm) 9.18 (br. s, 1H, NH), 8.18 (d, 1H), 7.94 (d, 1H), 7.59 (dd, 1H), 6.14 (br. s, 1H, NH), 4.32 (q, 2H), 1.39 (t, 3H).

### Example No. 1-34

¹H NMR (400 MHz, CDCl₃ δ, ppm) 8.87 (br. s, 1H, NH), 8.38 (d, 1H), 8.18 (d, 1H), 7.68 (dd, 1H), 5.95 (br. s, 1H, NH), 3.58 (q, 2H), 1.44 (d, 3H).

### Example No. 1-36

¹H NMR (400 MHz, d₆-DMSO δ, ppm) 14.05 (br. s, 1H, NH), 8.78 (br. s, 1H, NH), 8.09 (d, 1H), 7.90 (br. s, 1H, NH), 7.82 (d, 1H), 7.51 (dd, 1H), 6.42-6.20 (m, 1H).

### Example No. 1-37:

¹H NMR (400 MHz, d₆-DMSO δ, ppm) 13.72 (br. s, 1H, NH), 8.69 (br. s, 1H, NH), 7.98 (d, 1H), 7.92 (br. s, 1H, NH), 7.83 (d, 1H), 7.52 (dd, 1H).

### Example No. 1-38

¹H NMR (400 MHz, CD₃OD δ, ppm) 7.97 (d, 1H), 7.72 (d, 1H), 7.38 (dd, 1H), 4.12 (m, 1H), 1.71 (d, 3H).

### Example No. 1-39

¹H NMR (400 MHz, CD₃OD δ, ppm) 8.08 (d, 1H), 7.83 (d, 1H), 7.50 (dd, 1H).

### Example No. 1-40

¹H NMR (400 MHz, CD₃OD δ, ppm) 7.96 (d, 1H), 7.73 (d, 1H), 7.36 (dd, 1H), 3.96 (q, 2H).

### Example No. 1-41

¹H NMR (400 MHz, CD₃OD δ, ppm) 7.92 (d, 1H), 7.68 (d, 1H), 7.34 (dd, 1H), 1.93 (s, 3H), 1.87 (s, 3H).

### Example No. 1-42

¹H NMR (400 MHz, CD₃OD δ, ppm) 7.97 (d, 1H), 7.72 (d, 1H), 7.35 (dd, 1H), 5.88-6.07 (dq, 1H), 1.84 (dd, 3H).

### Example No. 1-43

¹H NMR (400 MHz, CD₃OD δ, ppm) 7.84 (d, 1H), 7.66 (d, 1H), 7.28 (dd, 1H), 1.33 (m, 1H), 0.91 (m, 2H).

### Example No. 1-44

¹H NMR (400 MHz, CD₃OD δ, ppm) 7.86 (d, 1H), 7.69 (d, 1H), 7.19 (dd, 1H), 2.73 (m, 2H), 2.58 (m, 1H), 2.24 (m, 1H).

### Example No. 1-45

¹H NMR (400 MHz, CD₃OD δ, ppm) 8.08 (d, 1H), 7.81 (d, 1H), 7.50 (dd, 1H), 4.42 (t, 2H), 1.87 (sext, 2H), 1.11 (t, 3H); ¹⁹F NMR (375 MHz, CD₃OD δ, ppm) −70.82.

### Example No. 1-46

¹H NMR (400 MHz, CD₃OD δ, ppm) 8.02 (d, 1H), 7.91 (d, 1H), 7.54 (dd, 1H).

### Example No. 1-47

¹H NMR (400 MHz, CDCl_{3 δ, ppm)} 11.52 (br. s, 1H, NH), 8.18 (d, 1H), 7.94 (d, 1H), 7.38 (dd, 1H), 3.30 (sept, 2H), 1.48 (d, 12H).

### Example No. 1-48

¹H NMR (400 MHz, CDCl₃ δ, ppm) 11.51 (br. s, 1H, NH), 8.17 (d, 1H), 7.92 (d, 1H), 7.36 (dd, 1H), 3.06 (m, 2H), 1.92 (m, 2H), 1.66 (m, 2H), 1.42 (m, 6H), 1.23 (t, 3H), 1.06 (t, 3H).

### Example No. 1-49

¹H NMR (400 MHz, CDCl₃ δ, ppm) 11.44 (br. s, 1H, NH), 8.14 (d, 1H), 7.95 (d, 1H), 7.37 (dd, 1H), 3.14 (m, 2H), 2.96 (m, 2H), 1.87 (sext, 4H), 1.14 (t, 6H).

### Example No. 1-50

¹H NMR (400 MHz, CD₃OD δ, ppm) 8.06 (d, 1H), 7.82 (d, 1H), 7.51 (dd, 1H), 4.48 (t, 2H), 1.84 (quint, 2H), 1.53 (sext, 2H), 1.02 (t, 3H).

### Example No. 1-51

¹H NMR (400 MHz, CD₃OD δ, ppm) 8.04 (d, 1H), 7.79 (d, 1H), 7.46 (dd, 1H), 4.22 (q, 2H), 1.42 (t, 3H).

### Example No. 1-52

¹H NMR (400 MHz, CD₃OD δ, ppm) 8.09 (d, 1H), 7.81 (d, 1H), 7.49 (dd, 1H), 4.29 (m, 2H), 3.74 (m, 2H), 3.45 (s, 3H).

### Example No. 1-53

¹H NMR (400 MHz, CDCl₃ δ, ppm) 9.12 (br. s, NH), 8.28 (d, 1H), 7.64 (d, 1H), 7.58 (dd, 1H), 5.90 (br. s, NH), 4.07 (s, 3H).

### Example No. 1-54

¹H NMR (400 MHz, CDCl₃ δ, ppm) 9.14 (br. s, NH), 8.27 (d, 1H), 7.64 (d, 1H), 7.55 (dd, 1H), 5.86 (br. s, NH), 4.48 (q, 2H), 1.56 (t, 3H).

### Example No. 1-55

¹H NMR (400 MHz, CDCl₃ δ, ppm) 8.88 (br. s, NH), 8.37 (d, 1H), 7.73 (d, 1H), 7.71 (dd, 1H), 5.92 (br. s, NH), 5.31 (s, 2H).

### Example No. 1-56

¹H NMR (400 MHz, CDCl₃ δ, ppm) 9.04 (br. s, NH), 8.31 (d, 1H), 7.80 (d, 1H), 7.62 (dd, 1H), 5.89 (br. s, NH), 5.18 (d, 2H), 2.46 (t, 1H).

### Example No. 1-62

¹H NMR (400 MHz, CDCl₃ δ, ppm) 9.21 (br. s, NH), 8.26 (d, 1H), 7.62 (d, 1H), 7.53 (dd, 1H), 5.88 (br. s, NH), 4.40 (q, 2H), 1.52 (t, 3H).

### Example No. 1-63

¹H NMR (400 MHz, CDCl₃ δ, ppm) 8.92 (br. s, NH), 8.38 (d, 1H), 7.72 (d, 1H), 7.68 (dd, 1H), 5.93 (br. s, NH), 5.27 (s, 2H).

### Example No. 1-64

¹H NMR (400 MHz, CDCl₃ δ, ppm) 9.11 (br. s, NH), 8.32 (d, 1H), 7.78 (d, 1H), 7.60 (dd, 1H), 5.91 (br. s, NH), 5.14 (d, 2H), 2.48 (m, 1H).

### Example No. 1-65

¹H NMR (400 MHz, CDCl₃ δ, ppm) 9.08 (br. s, NH), 8.31 (d, 1H), 7.69 (d, 1H), 7.61 (dd, 1H), 6.26-5.98 (tt, 1H), 5.90 (br. s, NH), 4.70 (dq, 2H).

### Example No. 1-69

¹H NMR (400 MHz, CDCl₃ δ, ppm) 9.22 (br. s, NH), 8.27 (d, 1H), 7.63 (d, 1H), 7.54 (dd, 1H), 5.87 (br. s, NH), 4.34 (t, 2H), 1.86 (quint, 2H), 1.43 (sext, 2H), 0.98 (t, 3H).

### Example No. 1-71

¹H NMR (400 MHz, CDCl₃ δ, ppm) 9.16 (br. s, NH), 8.29 (d, 1H), 7.65 (d, 1H), 7.58 (dd, 1H), 5.88 (br. s, NH), 4.04 (s, 3H).

### Example No. 1-72

¹H NMR (400 MHz, CDCl₃ δ, ppm) 9.18 (br. s, NH), 8.27 (d, 1H), 7.64 (d, 1H), 7.56 (dd, 1H), 5.86 (br. s, NH), 4.44 (q, 2H), 1.53 (t, 3H).

### Example No. 1-73

¹H NMR (400 MHz, CDCl₃ δ, ppm) 8.91 (br. s, NH), 8.39 (d, 1H), 7.74 (d, 1H), 7.70 (dd, 1H), 5.92 (br. s, NH), 5.30 (s, 2H).

### Example No. 1-75

¹H NMR (400 MHz, CDCl₃ δ, ppm) 9.03 (br. s, NH), 8.34 (d, 1H), 7.69 (d, 1H), 7.63 (dd, 1H), 5.94 (br. s, NH), 4.96 (q, 2H).

### Example No. 1-77

¹H NMR (400 MHz, CDCl₃ δ, ppm) 9.02 (br. s, NH), 8.35 (d, 1H), 7.65 (d, 1H), 7.62 (dd, 2H), 5.91 (br. s, NH), 4.93 (t, 2H).

### Example No. 1-78

¹H NMR (400 MHz, CDCl₃ δ, ppm) 9.08 (br. s, NH), 8.34 (d, 1H), 7.68 (d, 1H), 7.61 (dd, 1H), 6.14-5.89 (tt, 1H), 5.93 (br. s, NH), 4.90 (t, 2H).

### Example No. 1-79

¹H NMR (400 MHz, CDCl₃ δ, ppm) 8.95 (br. s, NH), 8.36 (d, 1H), 7.68 (d, 1H), 7.64 (dd, 1H), 5.93 (br. s, NH), 4.98 (q, 2H).

### Example No. 1-82

¹H NMR (400 MHz, CDCl₃ δ, ppm) 8.99 (br. s, NH), 8.34 (d, 1H), 7.70 (d, 1H), 7.63 (dd, 1H), 6.14-5.88 (t, 1H), 5.92 (br. s, NH), 4.94 (t, 2H).

### Example No. 1-83

¹H NMR (400 MHz, d₆-DMSO δ, ppm) 14.57 (br. s, 1H, NH), 8.42 (br. s, 1H, NH), 7.95 (m, 1H), 7.87 (d, 1H), 7.54 (m, 1H), 4.49 (m, 1H), 2.33 (m, 2H), 2.01 (m, 2H), 1.72 (m, 2H).

### Example No. 1-84

¹H NMR (400 MHz, CDCl₃ δ, ppm) 9.37 (br. s, 1H, NH), 8.06 (d, 1H), 7.71 (d, 1H), 7.49 (dd, 1H), 5.93 (br. s, 2H, NH), 3.98 (d, 2H), 1.27 (m, 1H), 0.68 (m, 2H), 0.38 (m, 2H).

### Example No. 1-85

¹H NMR (400 MHz, CD₃OD δ, ppm) 8.08 (d, 1H), 7.80 (d, 1H), 7.49 (dd, 1H), 4.03 (dd, 1H), 3.98 (dd, 1H), 1.83 (m, 1H), 1.59 (m, 1H), 1.32 (m, 1H), 1.05 (m, 2H), 0.99 (m, 2H).

### Example No. 1-86

¹H NMR (400 MHz, CDCl₃ δ, ppm) 9.31 (br. s, 1H, NH), 8.07 (d, 1H), 7.70 (d, 1H), 7.50 (dd, 1H), 5.91 (br. s, 2H, NH), 4.25 (t, 2H), 2.70 (m, 2H), 2.08 (m, 1H).

### Example No. 1-87

¹H NMR (400 MHz, CD₃OD δ, ppm) 8.10 (d, 1H), 7.72 (d, 1H), 7.48 (dd, 1H), 4.29 (t, 2H), 2.42 (m, 2H), 2.31 (m, 1H), 2.01 (t, 2H).

### Example No. 1-88

¹H NMR (400 MHz, CD₃OD δ, ppm) 8.03 (d, 1H), 7.78 (d, 1H), 7.42 (dd, 1H), 3.73 (t, 2H), 2.50 (m, 2H), 2.12 (q, 2H), 1.09 (t, 3H).

### Example No. 1-89

¹H NMR (400 MHz, CD₃OD δ, ppm) 8.09 (d, 1H), 7.80 (d, 1H), 7.48 (dd, 1H), 5.91 (m, 1H), 5.21 (m, 1H), 5.13 (m, 1H), 4.19 (t, 2H), 2.56 (m, 2H).

### Example No. 1-90

¹H NMR (400 MHz, CD₃OD δ, ppm) 8.08 (d, 1H), 7.81 (d, 1H), 7.47 (dd, 1H), 4.12 (d, 2H), 2.79 (m, 1H), 2.18 (m, 2H), 1.92 (m, 6H).

### Example No. 1-91

¹H NMR (400 MHz, CD₃OD δ, ppm) 8.07 (d, 1H), 7.80 (d, 1H), 7.49 (dd, 1H), 4.23 (t, 2H), 3.59 (t, 2H), 3.38 (s, 3H), 2.06 (pent, 2H).

### Example No. 1-92

¹H NMR (400 MHz, CDCl₃ δ, ppm) 9.36 (br. s, 1H, NH), 8.06 (d, 1H), 7.69 (d, 1H), 7.49 (dd, 1H), 5.93 (br. s, 2H, NH), 4.11 (t, 2H), 1.78 (m, 2H), 1.61 (m, 1H), 1.31 (m, 2H), 0.93 (d, 3H), 0.90 (d, 3H).

### Example No. 1-94

¹H NMR (400 MHz, CD₃OD δ, ppm) 8.08 (d, 1H), 7.82 (d, 1H), 7.51 (dd, 1H), 4.71 (q, 2H).

### Example No. 1-95

¹H NMR (400 MHz, CD₃OD δ, ppm) 8.07 (d, 1H), 7.81 (d, 1H), 7.49 (dd, 1H), 4.41 (t, 2H), 2.72 (m, 2H).

### Example No. 1-96

¹H NMR (400 MHz, CD₃OD δ, ppm) 8.08 (d, 1H), 7.80 (d, 1H), 7.47 (dd, 1H), 4.24 (t, 2H), 2.41 (m, 2H), 2.07 (m, 2H).

### Example No. 1-99

¹H NMR (400 MHz, CDCl₃ δ, ppm) 9.21 (br. s, 1H, NH), 8.08 (d, 1H), 7.72 (d, 1H), 7.42 (dd, 1H), 5.96 (br. s, 2H, NH), 5.13 (m, 1H), 4.58 (m, 1H), 4.49 (m, 1H).

### Example No. 1-100

¹H NMR (400 MHz, CDCl_{3 δ, ppm)} 9.40 (br. s, 1H, NH), 8.23 (d, 1H), 7.70 (d, 1H), 7.48 (dd, 1H), 6.04 (br. s, 2H, NH), 4.02 (d, 2H), 2.33 (m, 1H), 1.82 (m, 2H), 1.73 (m, 2H), 1.64 (m, 2H), 1.33 (m, 2H).

### Example No. 1-101

¹H NMR (400 MHz, CDCl₃ δ, ppm) 9.44 (br. s, 1H, NH), 8.10 (d, 1H), 7.82 (d, 1H), 7.43 (dd, 1H), 6.41 (br. s, 2H, NH), 6.18-5.91 (tt, 1H), 4.31 (dt, 2H).

### Example No. 1-102

¹H NMR (400 MHz, CDCl₃ δ, ppm) 9.22 (br. s, 1H, NH), 8.07 (d, 1H), 7.73 (d, 1H), 7.41 (dd, 1H), 6.10-5.84 (tt, 1H), 5.93 (br. s, 2H, NH), 4.52 (m, 2H).

### Example No. 1-103

¹H NMR (400 MHz, CDCl₃ δ, ppm) 9.35 (br. s, 1H, NH), 8.05 (d, 1H), 7.71 (d, 1H), 7.42 (dd, 1H), 5.95 (br. s, 2H, NH), 3.93 (d, 2H), 3.71 (m, 1H), 3.12 (m, 1H), 2.43 (m, 1H), 2.11 (m, 1H), 1.91 (m, 2H), 1.80 (m, 1H), 1.69 (m, 1H), 1.38 (m, 1H), 1.22 (s, 3H), 0.88 (s, 3H).

### Example No. 2-2

¹H NMR (400 MHz, d₆-DMSO δ, ppm) 14.34 (br. s, 1H, NH), 8.52 (br. s, 1H, NH), 8.09 (d, 1H), 7.97 (br. s, 1H, NH), 7.89 (m, 1H).

### Example No. 2-7

¹H NMR (400 MHz, d₆-DMSO δ, ppm) 14.12 (br. s, 1H, NH), 8.77 (br. s, 1H, NH), 8.02 (d, 1H), 7.95 (br. s, 1H, NH), 7.86 (m, 1H), 7.33-7.20 (t, 1H).

### Example No. 3-2

¹H NMR (400 MHz, d₆-DMSO δ, ppm) 13.87 (br. s, 1H, NH), 8.82 (br. s, 1H, NH), 8.35 (s, 1H), 8.19 (s, 1H), 7.79 (br. s, 1H, NH).

### Example No. 3-14

¹H NMR (400 MHz, d₆-DMSO δ, ppm) 13.82 (br. s, 1H, NH), 8.76 (br. s, 1H, NH), 8.32 (s, 1H), 8.22 (s, 1H), 7.84 (br. s, 1H, NH).

### Example No. 3-17

¹H NMR (400 MHz, d₆-DMSO δ, ppm) 13.58 (br. s, 1H, NH), 8.60 (br. s, 1H, NH), 8.27 (s, 1H), 8.21 (s, 1H), 8.05 (br. s, 1H, NH).

### Example No. 3-25

¹H NMR (400 MHz, CDCl₃ δ, ppm) 8.92 (br. s, 1H, NH), 8.39 (s, 1H), 8.32 (s, 1H), 6.29 (br. s, 1H, NH), 3.95 (sept, 1H), 1.50 (d, 6H).

### Example No. 3-27

¹H NMR (400 MHz, d₆-DMSO δ, ppm) 8.96 (br. s, 1H, NH), 8.60 (s, 1H), 8.56 (br. s, 1H, NH), 8.19 (s, 1H), 4.14 (s, 3H).

### Example No. 3-28

¹H NMR (400 MHz, d₆-DMSO δ, ppm) 9.02 (br. s, 1H, NH), 8.57 (s, 1H), 8.29 (br. s, 1H, NH), 8.20 (s, 1H), 2.73 (s, 3H).

### Example No. 3-35

¹H NMR (400 MHz, CDCl₃ δ, ppm) 8.81 (br. s, 1H, NH), 8.69 (s, 1H), 8.50 (s, 1H), 6.19 (br. s, 1H, NH), 3.54 (s, 3H).

### Example No. 3-62

¹H NMR (400 MHz, CDCl₃ δ, ppm) 9.12 (br. s, 1H, NH), 8.56 (s, 1H), 7.92 (s, 1H), 6.02 (br. s, 1H, NH), 4.47 (q, 2H), 1.59 (t, 3H).

### Example No. 4-1

¹H NMR (400 MHz, d₆-DMSO δ, ppm) 14.21 (br. s, 1H, NH), 8.62 (br. s, 1H, NH), 7.80 (br. s, 1H, NH), 7.77 (m, 1H), 7.61 (m, 1H), 2.58 (s, 3H) ; ¹⁹F NMR (375 MHz, CD₃OD δ, ppm) −53.1.

### Example No. 4-2

¹H NMR (400 MHz, CDCl₃ δ, ppm) 14.46 (br. s, 1H, NH), 8.63 (br. s, 1H, NH), 7.89 (br. s, 1H, NH), 7.82 (d, 1H), 7.67 (m, 1H), 2.58 (s, 3H).

### Example No. 4-7

¹H NMR (400 MHz, CDCl₃ δ, ppm) 13.72 (br. s, 1H, NH), 8.85 (br. s, 1H, NH), 7.82 (br. s, 1H, NH), 7.79 (d, 1H), 7.59 (m, 1H), 7.50-7.22 (t, 1H), 2.54 (s, 3H).

### Example No. 5-2

¹H NMR (400 MHz, CD₃OD δ, ppm) 7.72 (br. m, 1H, NH), 7.44 (m, 2H);

### Example No. 5-8

¹H NMR (400 MHz, CD₃OD δ, ppm) 7.65 (m, 1H), 7.51 (br. m, 1H, NH), 7.40 (m, 1H), 6.48-6.30 (dq, 1H).

### Example No. 5-14

¹H NMR (400 MHz, d₆-DMSO δ, ppm) 14.71 (br. s, 1H, NH), 8.61 (br. s, 1H, NH), 7.72 (m, 2H); ¹⁹F NMR (375 MHz, CD₃OD δ, ppm) −85.2, −116.5.

### Example No. 5-15

¹H NMR (400 MHz, d₆-DMSO δ, ppm) 14.80 (br. s, 1H, NH), 9.19 (br. s, 1H, NH), 7.71 (m, 2H), 2.93 (m, 1H), 0.80 (m, 2H), 0.61 (m, 2H).

### Example No. 5-17

¹H NMR (400 MHz, CD₃OD δ, ppm) 7.74 (dd, 1H), 7.62 (dd, 1H); ¹⁹F NMR (375 MHz, CD₃OD δ, ppm) −82.3, −119.2, −121.8, −128.3.

### Example No. 5-21

¹H NMR (400 MHz, CDCl_{3 δ, ppm)} 9.19 (br. s, 1H, NH), 8.03 (dd, 1H), 7.71 (dd, 1H), 6.11 (br. s, 1H, NH), 4.33 (sept, 1H), 1.34 (d, 6H).

### Example No. 5-24

¹H NMR (400 MHz, CDCl₃ δ, ppm) 9.39 (br. s, 1H, NH), 8.05 (m, 1H), 7.73 (m, 1H), 6.41 (br. s, 1H, NH), 5.99 (m, 1H), 5.33 (m, 1H), 5.20 (m, 1H), 4.22 (m, 1H), 4.14 (m, 1H).

### Example No. 6-21

¹H NMR (400 MHz, CDCl₃ δ, ppm) 9.98 (br. s, 1H, NH), 7.35 (dd, 1H), 7.30 (d, 1H), 6.09 (br. s, 1H, NH), 4.29 (sept, 1H), 1.32 (d, 6H).

### Example No. 6-24

¹H NMR (400 MHz, CDCl₃ δ, ppm) 10.03 (br. s, 1H, NH), 7.38 (m, 1H), 7.34 (m, 1H), 6.38 (br. s, 1H, NH), 5.90 (m, 1H), 5.39 (m, 1H), 5.27 (m, 1H), 4.24 (m, 1H), 4.16 (m, 1H).

### Example No. 7-2

¹H NMR (400 MHz, CD₃OD δ, ppm) 8.21 (m, 1H), 8.18 (m, 1H).

### Example No. 7-8

¹H NMR (400 MHz, CD₃OD δ, ppm) 8.53 (m, 1H), 8.39 (m, 1H), 6.47-6.35 (dq, 1H).

### Example No. 7-14

¹H NMR (400 MHz, CD₃OD δ, ppm) 8.05 (m, 1H), 7.93 (br. s, 1H, NH), 7.88 (m, 1H).

The present invention accordingly provides for the use of at least one compound selected from the group consisting of fluoroalkyl-substituted 2-amidobenzimidazoles of the formula (I), and of any desired mixtures of these inventive fluoroalkyl-substituted 2-amidobenzimidazoles of the formula (I), with active agrochemical ingredients in accordance with the definition below, for enhancement of the resistance of plants to abiotic stress factors, especially for strengthening of plant growth and/or for increasing the plant yield.

The present invention further provides a spray solution for treatment of plants, comprising an amount, effective for enhancement of the resistance of plants to abiotic stress factors, of at least one compound selected from the group consisting of fluoroalkyl-substituted 2-amidobenzimidazoles of the formula (I). The abiotic stress conditions which can be relativized may include, for example, drought, cold and hot conditions, osmotic stress, waterlogging, elevated soil salinity, elevated exposure to minerals, ozone conditions, strong light conditions, limited availability of nitrogen nutrients, limited availability of phosphorus nutrients.

In one embodiment, for example, the compounds provided in accordance with the invention, the fluoroalkyl-substituted 2-amidobenzimidazoles, may be applied by spray application to appropriate plants or parts of plants to be treated. The inventive compounds (I) are used as envisaged in accordance with the invention preferably with a dosage between 0.0005 and 3 kg/ha, more preferably between 0.001 and 2 kg/ha, especially preferably between 0.005 and 1 kg/ha. If, in the context of the present invention, abscisic acid is used simultaneously with fluoroalkyl-substituted 2-amidobenzimidazoles, for example in the context of a combined preparation or formulation, abscisic acid is preferably added in a dosage between 0.001 and 3 kg/ha, more preferably between 0.005 and 2 kg/ha, especially preferably between 0.01 and 1 kg/ha.

The term “resistance to abiotic stress” is understood in the context of the present invention to mean various kinds of advantages for plants. Such advantageous properties are manifested, for example, in the following improved plant characteristics: improved root growth with regard to surface area and depth, increased stolon and tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soil and water, enhanced tolerance to ozone stress, improved compatibility with respect to herbicides and other crop treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

More particularly, the inventive use exhibits the advantages described in spray application to plants and plant parts. Combinations of the corresponding fluoroalkyl-substituted 2-amidobenzimidazoles of the formula (I) with substances including insecticides, attractants, acaricides, fungicides, nematicides, herbicides, growth regulators, safeners, substances which influence plant maturity, and bactericides can likewise be employed in the control of plant disorders in the context of the present invention. In addition, the combined use of corresponding fluoroalkyl-substituted 2-amidobenzimidazoles of the formula (I) with genetically modified cultivars with a view to increased tolerance to abiotic stress is likewise possible.

As is well known, some of the different kinds of advantages mentioned above for plants can be combined, and are documented by generally accepted terms. Such terms are, for example, the following designations: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms which are well known to a person skilled in the art.

In the context of the present invention, a good effect on resistance to abiotic stress is understood to mean, without limitation,
at least an emergence improved by generally 3%, especially more than 5%, preferably more than 10%,
at least a yield enhanced by generally 3%, especially more than 5%, preferably more than 10%,
at least a root development improved by generally 3%, especially more than 5%, preferably more than 10%,
at least a shoot size rising by generally 3%, especially more than 5%, preferably more than 10%,
at least a leaf area increased by generally 3%, especially more than 5%, preferably more than 10%,
at least an emergence improved by generally 3%, especially more than 5%, preferably more than 10%,
at least a photosynthesis performance improved by generally 3%, especially more than 5%, preferably more than 10%, and/or
at least a flower development improved by generally 3%, especially more than 5%, preferably more than 10%,
   it being possible for the effects to manifest themselves individually or else in any combination of two or more effects.

The present invention further provides a spray solution for treatment of plants, comprising an amount, effective for enhancement of the resistance of plants to abiotic stress factors, of at least one compound of the formula (I). The spray solution may comprise other customary constituents, such as solvents, formulation aids, especially water. Further constituents may include active agrochemical ingredients described below.

The present invention further provides for the use of corresponding spray solutions for increasing the resistance of plants to abiotic stress factors. The remarks which follow apply both to the inventive use of the compounds of the formula (I) per se and to the corresponding spray solutions.

In accordance with the invention, it has additionally been found that the application, to plants or in their environment, of the compounds of the formula (I) in combination with at least one fertilizer as defined below is possible.

Fertilizers which can be used in accordance with the invention together with the compounds of the formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention should be made of the NPK fertilizers, i.e. fertilizers which contain nitrogen, phosphorus and potassium, calcium ammonium nitrate, i.e. fertilizers which additionally contain calcium, or ammonium nitrate sulfate (formula (NH₄)₂SO₄ NH₄NO₃), ammonium phosphate and ammonium sulfate. These fertilizers are generally known to the person skilled in the art; see also, for example, Ullmann's Encyclopedia of Industrial Chemistry, 5th edition, vol. A 10, pages 323 to 431, Verlagsgesellschaft, Weinheim, 1987.

The fertilizers may also contain salts of micronutrients (preferably calcium, sulfur, boron, manganese, magnesium, iron, boron, copper, zinc, molybdenum and cobalt) and phytohormones (for example vitamin B1 and indole-3-acetic acid) or mixtures thereof. Fertilizers used in accordance with the invention may also contain further salts, such as monoammonium phosphate (MAP), diammonium phosphate (DAP), potassium sulfate, potassium chloride, magnesium sulfate. Suitable amounts of the secondary nutrients, or trace elements, are amounts of 0.5 to 5% by weight, based on the overall fertilizer. Further possible ingredients are crop protection compositions, insecticides or fungicides, growth regulators or mixtures thereof. This will be explained in more detail below.

The fertilizers can be used, for example, in the form of powders, granules, prills or compactates. However, the fertilizers can also be used in liquid form, dissolved in an aqueous medium. In this case, it is also possible to use dilute aqueous ammonia as the nitrogen fertilizer. Further possible constituents of fertilizers are described, for example, in Ullmann's Encyclopedia of Industrial Chemistry, 5th edition, 1987, Vol. A 10, pages 363 to 401, DE-A 41 28 828, DE-A 19 05 834 and DE-A 196 31 764. The general composition of the fertilizers which, in the context of the present invention, may take the form of straight and/or compound fertilizers, for example composed of nitrogen, potassium or phosphorus, may vary within a wide range. In general, a content of 1 to 30% by weight of nitrogen (preferably 5 to 20% by weight), 1 to 20% by weight of potassium (preferably 3 to 15% by weight) and a content of 1 to 20% by weight of phosphorus (preferably 3 to 10% by weight) is advantageous. The microelement content is typically in the ppm range, preferably in the range from 1 to 1000 ppm. In the context of the present invention, the fertilizer and the compounds of the formula (I) may be administered simultaneously, i.e. synchronously. However, it is also possible first to apply the fertilizer and then a compound of the formula (I), or first to apply a compound of the formula (I) and then the fertilizer. In the case of nonsynchronous application of a compound of the formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, the inventive compound of the formula (I) and the fertilizer are applied within a time frame of less than 1 hour, preferably less than 30 minutes, more preferably less than 15 minutes.

The active ingredients for use in accordance with the invention, if appropriate in combination with fertilizers, can preferably be employed in the following plants, the enumeration which follows being nonlimiting.

Preferred plants are those from the group of the useful plants, ornamentals, turfs, commonly used trees employed as ornamentals in the public and domestic sectors, and forestry trees. Forestry trees include trees for the production of timber, cellulose, paper and products made from parts of the trees. The term “useful plants” as used here refers to crop plants which are employed as plants for obtaining foods, animal feeds, fuels or for industrial purposes.

The useful plants include, for example, the following types of plants: triticale, durum (hard wheat), turf, vines, cereals, for example wheat, barley, rye, oats, hops, rice, corn and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao beans and peanuts; cucurbits, for example pumpkin/squash, cucumbers and melons; fiber plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamonum, camphor, or also plants such as tobacco, nuts, coffee, eggplant, sugarcane, tea, pepper, grapevines, hops, bananas, latex plants and ornamentals, for example flowers, shrubs, deciduous trees and coniferous trees. This enumeration does not constitute a limitation.

The following plants are considered to be particularly suitable target crops for the application of the inventive method: oats, rye, triticale, durum, cotton, eggplant, turf, pome fruit, stone fruit, soft fruit, corn, wheat, barley, cucumber, tobacco, vines, rice, cereals, pear, pepper, beans, soybeans, oilseed rape, tomato, bell pepper, melons, cabbage, potatoes and apples.

Examples of trees which can be improved in accordance with the inventive method include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved in accordance with the inventive method include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved in accordance with the inventive method include: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved in accordance with the inventive method include: horse chestnut, Platanaceae, linden tree, maple tree.

The present invention can also be applied to any turf grasses, including cool-season turf grasses and warm-season turf grasses. Examples of cool-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festuca capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turfgrasses are Bermudagrass (Cynodon spp. L. C. Rich), zoysiagrass (Zoysia spp. Willd.), St. Augustine grass (Stenotaphrum secundatum Walt Kuntze), centipedegrass (Eremochloa ophiuroides Munro Hack.), carpetgrass (Axonopus affinis Chase), Bahia grass (Paspalum notatum Flugge), Kikuyugrass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.). Cool-season turfgrasses are generally preferred for the use in accordance with the invention. Especially preferred are bluegrass, bentgrass and redtop, fescues and ryegrasses. Bentgrass is especially preferred.

Particular preference is given in accordance with the invention to treating plants of the plant cultivars which are in each case commercially available or in use. Plant cultivars are understood to mean plants which have new properties (“traits”) and which have been obtained by conventional breeding, by mutagenesis or with the aid of recombinant DNA techniques. Crop plants may accordingly be plants which can be obtained by conventional breeding and optimization methods or by biotechnological and genetic engineering methods or combinations of these methods, including the transgenic plants and including the plant varieties which can and cannot be protected by plant breeders' rights.

The inventive treatment method can thus also be used for the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNAi technology [RNA interference]). A heterologous gene that is located in the genome is also called a transgene. A transgene that is defined by its particular location in the plant genome is called a transformation or transgenic event.

Plants and plant varieties which are preferably treated according to the invention include all plants which have genetic material which imparts particularly advantageous, useful traits to these plants (whether obtained by breeding and/or biotechnological means).

Plants and plant varieties which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can also be affected by improved plant architecture (under stress and non-stress conditions), including early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may likewise be treated according to the invention are hybrid plants that already express the characteristics of heterosis, or hybrid vigor, which results in generally higher yield, vigor, health and resistance toward biotic and abiotic stress factors. Such plants are typically made by crossing an inbred male-sterile parent line (the female parent) with another inbred male-fertile parent line (the male parent). Hybrid seed is typically harvested from the male-sterile plants and sold to growers. Male-sterile plants can sometimes (e.g. in corn) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species (WO 1992/005251, WO 1995/009910, WO 1998/27806, WO 2005/002324, WO 2006/021972 and US 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar (e.g. WO 1991/002069).

Plants or plant varieties (obtained by plant biotechnology methods such as genetic engineering) which may also be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 2001/66704). It can also be a mutated EPSPS, as described, for example, in EP-A 0837944, WO 2000/066746, WO 2000/066747 or WO 2002/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyltransferase enzyme as described, for example, in WO 2002/036782, WO 2003/092360, WO 2005/012515 and WO 2007/024782. Glyphosate-tolerant plants can also be obtained by selecting plants containing naturally occurring mutations of the above-mentioned genes as described, for example, in WO 2001/024615 or WO 2003/013226.

Other herbicide-resistant plants are for example plants which have been made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species for example). Plants expressing an exogenous phosphinothricin acetyltransferase have been described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD inhibitors can be transformed with a gene encoding a naturally occurring resistant HPPD enzyme, or a gene encoding a mutated HPPD enzyme according to WO 1996/038567, WO 1999/024585 and WO 1999/024586. Tolerance to HPPD inhibitors can also be obtained by transforming plants with genes encoding certain enzymes enabling the formation of homogentisate despite the inhibition of the native HPPD enzyme by the HPPD inhibitor. Such plants and genes are described in WO 1999/034008 and WO 2002/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928.

Further herbicide-resistant plants are plants that have been made tolerant to acetolactate synthase (ALS) inhibitors. Known ALS inhibitors include, for example, sulfonylurea, imidazolinone, triazolopyrimidines, pyrimidinyl oxy(thio)benzoates, and/or sulfonylaminocarbonyltriazolinone herbicides. Different mutations in the ALS enzyme (also known as acetohydroxy acid synthase, AHAS) are known to confer tolerance to different herbicides and groups of herbicides, as described, for example, in Tranel and Wright, Weed Science (2002), 50, 700-712, and also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 1996/033270. Further imidazolinone-tolerant plants have also been described, for example in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example in WO 2007/024782.

Other plants tolerant to imidazolinone and/or sulfonylurea can be obtained by induced mutagenesis, by selection in cell cultures in the presence of the herbicide or by mutation breeding, as described, for example, for soybeans in U.S. Pat. No. 5,084,082, for rice in WO 1997/41218, for sugar beet in U.S. Pat. No. 5,773,702 and WO 1999/057965, for lettuce in U.S. Pat. No. 5,198,599 or for sunflower in WO 2001/065922.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or

2) a crystal protein from Bacillus thuringiensis or a portion thereof which is insecticidal in the presence of a second crystal protein other than Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or

3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by corn event MON98034 (WO 2007/027777); or

4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in corn events MON863 or MON88017, or the Cry3A protein in corn event MIR604; or

5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html or

6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 1994/21795); or

7) a hybrid insecticidal protein comprising parts from different secreted proteins from Bacillus thuringiensis or Bacillus cereus, such as a hybrid of the proteins in 1) above or a hybrid of the proteins in 2) above; or

8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5;

b. plants which contain a stress tolerance-enhancing transgene capable of reducing the expression and/or the activity of the PARG encoding genes of the plants or plant cells, as described, for example, in WO 2004/090140;

c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase, as described, for example, in EP 04077624.7 or WO 2006/133827 or PCT/EP07/002433.

Plants or plant varieties (obtained by plant biotechnology methods such as genetic engineering) which may also be treated according to the invention show altered quantity, quality and/or storage stability of the harvested product and/or altered properties of specific ingredients of the harvested product such as, for example:

1) Transgenic plants which synthesize a modified starch which is altered with respect to its chemophysical traits, in particular the amylose content or the amylose/amylopectin ratio, the degree of branching, the average chain length, the distribution of the side chains, the viscosity behavior, the gel resistance, the grain size and/or grain morphology of the starch in comparison to the synthesized starch in wild-type plant cells or plants, such that this modified starch is better suited for certain applications. These transgenic plants synthesizing a modified starch are described, for example, in EP 0571427, WO 1995/004826, EP 0719338, WO 1996/15248, WO 1996/19581, WO 1996/27674, WO 1997/11188, WO 1997/26362, WO 1997/32985, WO 1997/42328, WO 1997/44472, WO 1997/45545, WO 1998/27212, WO 1998/40503, WO 99/58688, WO 1999/58690, WO 1999/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 1995/26407, WO 1996/34968, WO 1998/20145, WO 1999/12950, WO 1999/66050, WO 1999/53072, U.S. Pat. No. 6,734,341, WO 2000/11192, WO 1998/22604, WO 1998/32326, WO 2001/98509, WO 2001/98509, WO 2005/002359, U.S. Pat. No. 5,824,790, U.S. Pat. No. 6,013,861, WO 1994/004693, WO 1994/009144, WO 1994/11520, WO 1995/35026 and WO 1997/20936.

2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, as described in EP 0663956, WO 1996/001904, WO 1996/021023, WO 1998/039460 and WO 1999/024593, plants which produce alpha-1,4-glucans, as described in WO 1995/031553, US 2002/031826, U.S. Pat. No. 6,284,479, U.S. Pat. No. 5,712,107, WO 1997/047806, WO 1997/047807, WO 1997/047808 and WO 2000/14249, plants which produce alpha-1,6-branched alpha-1,4-glucans, as described in WO 2000/73422, and plants which produce alternan, as described in WO 2000/047727, EP 06077301.7, U.S. Pat. No. 5,908,975 and EP 0728213.

3) Transgenic plants which produce hyaluronan, as described, for example, in WO 2006/032538, WO 2007/039314, WO 2007/039315, WO 2007/039316, JP 2006/304779 and WO 2005/012529.

Plants or plant varieties (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:

a) plants, such as cotton plants, which contain an altered form of cellulose synthase genes, as described in WO 1998/000549;

b) plants, such as cotton plants, which contain an altered form of rsw2 or rsw3 homologous nucleic acids, as described in WO 2004/053219;

c) plants, such as cotton plants, with an increased expression of sucrose phosphate synthase, as described in WO 2001/017333;

d) plants, such as cotton plants, with an increased expression of sucrose synthase, as described in WO 02/45485;

e) plants, such as cotton plants, wherein the timing of the plasmodesmatal gating at the basis of the fiber cell is altered, for example through downregulation of fiber-selective β-1,3-glucanase, as described in WO 2005/017157;

f) plants, such as cotton plants, which have fibers with altered reactivity, for example through the expression of the N-acetylglucosaminetransferase gene including nodC and chitin synthase genes, as described in WO 2006/136351.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;

b) plants, such as oilseed rape plants, which produce oil having a low linolenic acid content, as described in U.S. Pat. No. 6,270,828, U.S. Pat. No. 6,169,190 or U.S. Pat. No. 5,965,755;

c) plants, such as oilseed rape plants, which produce oil having a low level of saturated fatty acids, as described, for example, in U.S. Pat. No. 5,434,283.

Particularly useful transgenic plants which may be treated according to the invention are plants which comprise one or more genes which encode one or more toxins and are the transgenic plants available under the following trade names: YIELD GARD® (for example corn, cotton, soybeans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants which may be mentioned are corn varieties, cotton varieties and soybean varieties which are available under the following trade names: Roundup Ready® (tolerance to glyphosate, for example corn, cotton, soybeans), Liberty Link® (tolerance to phosphinothricin, for example oilseed rape), IMI® (tolerance to imidazolinone) and SOS® (tolerance to sulfonylurea, for example corn). Herbicide-resistant plants (plants bred in a conventional manner for herbicide tolerance) which may be mentioned include the varieties sold under the name Clearfield® (for example corn).

Particularly useful transgenic plants which may be treated according to the invention are plants containing transformation events, or a combination of transformation events, and that are listed for example in the databases for various national or regional regulatory agencies.

The compounds of the formula (I) to be used in accordance with the invention can be converted to customary formulations, such as solutions, emulsions, wettable powders, water- and oil-based suspensions, powders, dusts, pastes, soluble powders, soluble granules, granules for broadcasting, suspoemulsion concentrates, natural compounds impregnated with active ingredient, synthetic substances impregnated with active ingredient, fertilizers, and also microencapsulations in polymeric substances. In the context of the present invention, it is especially preferred when the inventive compounds of the formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing the compounds of the formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are prepared either in suitable equipment or else before or during application.

The auxiliaries used may be those substances which are suitable for imparting, to the composition itself and/or to preparations derived therefrom (for example spray liquors), particular properties such as particular technical properties and/or else special biological properties. Useful typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender used is water, it is also possible to use, for example, organic solvents as auxiliary solvents. Useful liquid solvents are essentially: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulfoxide, and also water.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Useful wetting agents which may be present in the formulations usable in accordance with the invention are all substances which promote wetting and which are conventionally used for the formulation of active agrochemical ingredients. Preference is given to using alkyl naphthalenesulfonates, such as diisopropyl or diisobutyl naphthalenesulfonates.

Useful dispersants and/or emulsifiers which may be present in the formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Suitable nonionic dispersants are especially ethylene oxide/propylene oxide block polymers, alkylphenol polyglycol ethers and tristryrylphenol polyglycol ether, and the phosphated or sulfated derivatives thereof. Suitable anionic dispersants are especially lignosulfonates, salts of polyacrylic acid and arylsulfonate/formaldehyde condensates.

Antifoams which may be present in the formulations usable in accordance with the invention are all foam-inhibiting substances conventionally used for the formulation of active agrochemical ingredients. Usable with preference are silicone antifoams and magnesium stearate.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preference is given to cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose.

Gibberellins which may be present in the formulations usable in accordance with the invention may preferably be gibberellins A1, A3 (=gibberellic acid), A4 and A7; particular preference is given to using gibberellic acid. The gibberellins are known (cf. R. Wegler “Chemie der Pflanzenschutz- and Schadlingsbekampfungsmittel” [Chemistry of Crop Protection Compositions and Pesticides], vol. 2, Springer Verlag, 1970, p. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01 and 98% by weight, preferably between 0.5 and 90%, of the compound of the formula (I).

The inventive active ingredient may be present in its commercially available formulations and in the use forms, prepared from these formulations, in a mixture with other active ingredients, such as insecticides, attractants, sterilants, bactericides, acaricides, nematicides, fungicides, growth regulators, herbicides, safeners, fertilizers or semiochemicals.

In addition, the described positive effect of the compounds of the formula (I) on the plants' own defenses can be supported by an additional treatment with active insecticidal, fungicidal or bactericidal ingredients.

Preferred times for the application of compounds of the formula (I) for enhancing resistance to abiotic stress are treatments of the soil, stems and/or leaves with the approved application rates.

The inventive compounds of the formula (I) may generally additionally be present in their commercial formulations and in the use forms prepared from these formulations in mixtures with other active ingredients, such as insecticides, attractants, sterilants, acaricides, nematicides, fungicides, growth regulators, substances which influence plant maturity, safeners or herbicides. Particularly suitable mixing partners are, for example, the active ingredients of the different classes, specified below in groups, without any preference resulting from the sequence thereof:

Fungicides:

F1) nucleic acid synthesis inhibitors, for example benalaxyl, benalaxyl-M, bupirimate, chiralaxyl, clozylacon, dimethirimol, ethirimol, furalaxyl, hymexazole, metalaxyl, metalaxyl-M, ofurace, oxadixyl, oxolinic acid;

F2) mitosis and cell division inhibitors, for example benomyl, carbendazim, diethofencarb, fuberidazole, fluopicolid, pencycuron, thiabendazole, thiophanate-methyl, zoxamide and chloro-7-(4-methylpiperidin-1-yl)-6-(2,4,6-trifluorophenyl)-[1,2,4]triazolo[1,5-a]pyrimidine;

F3) respiratory chain complex I/II inhibitors, for example diflumetorim, bixafen, boscalid, carboxin, diflumethorim, fenfuram, fluopyram, flutolanil, furametpyr, mepronil, oxycarboxin, penflufen, penthiopyrad, thifluzamid, N-[2-(1,3-dimethylbutyl)phenyl]-5-fluoro-1,3-dimethyl-1H-pyrazole-4-carboxamide, isopyrazam, sedaxan, 3-(difluoromethyl)-1-methyl-N-(3′,4′,5′-trifluorobiphenyl-2-yl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide and corresponding salts;

F4) respiratory chain complex III inhibitors, for example amisulbrom, azoxystrobin, cyazofamid, dimoxystrobin, enestrobin, famoxadon, fenamidon, fluoxastrobin, kresoxim-methyl, metominostrobin, orysastrobin, pyraclostrobin, pyribencarb, picoxystrobin, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(ethoxyimino)-N-methyl-2-(2-{[({(1E)-1-[3-(trifluoromethyl)-phenyl]ethylidene}amino)oxy]methyl}phenyl)ethanamide and corresponding salts, (2E)-2-(methoxyimino)-N-methyl-2-{2-[(E)-({1-[3-(trifluoromethyl)phenyl]ethoxy}-imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, 2-methyl {2-[({cyclopropyl[(4-methoxyphenyl)-imino]methyl}sulfanyl)methyl]phenyl}-3-methoxyacrylate, N-(3-ethyl-3,5,5-trimethylcyclohexyl)-3-(formylamino)-2-hydroxybenzamide and corresponding salts;

F5) decouplers, for example dinocap, fluazinam;

F6) ATP production inhibitors, for example fentin acetate, fentin chloride, fentin hydroxide, silthiofam

F7) amino acid and protein biosynthesis inhibitors, for example andoprim, blasticidin-S, cyprodinil, kasugamycin, kasugamycin hydrochloride hydrate, mepanipyrim, pyrimethanil

F8) signal transduction inhibitors, for example fenpiclonil, fludioxonil, quinoxyfen

F9) lipid and membrane synthesis inhibitors, for example chlozolinate, iprodione, procymidone, vinclozolin, ampropylfos, potassium-ampropylfos, edifenphos, iprobenfos (IBP), isoprothiolane, pyrazophos, tolclofos-methyl, biphenyl, iodocarb, propamocarb, propamocarb hydrochloride

F10) ergosterol biosynthesis inhibitors, for example fenhexamid, azaconazole, bitertanol, bromuconazole, diclobutrazole, difenoconazole, diniconazole, diniconazole-M, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, spiroxamine, tebuconazole, triadimefon, triadimenol, triticonazole, uniconazole, voriconazole, imazalil, imazalil sulfate, oxpoconazole, fenarimol, flurprimidol, nuarimol, pyrifenox, triforin, pefurazoat, prochloraz, triflumizole, viniconazole, aldimorph, dodemorph, dodemorph acetate, fenpropimorph, tridemorph, fenpropidin, naftifin, pyributicarb, terbinafin, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethyl-silyl)propoxy]phenyl}-N-ethyl-N-methylimidoformamide, N-ethyl-N-methyl-N′-{2-methyl-5-(trifluoromethyl)-4-(3-(trimethylsilyl)propoxy]phenyl}limidoformamide and O-{1-[(4-methoxyphenoxy)methyl]-2,2-dimethylpropyll-1H-imidazole-1-carbothioate;

F11) cell wall synthesis inhibitors, for example benthiavalicarb, bialaphos, dimethomorph, flumorph, iprovalicarb, polyoxins, polyoxorim, validamycin A

F12) melanine biosynthesis inhibitors, for example capropamide, diclocymet, fenoxanil, phthalide, pyroquilon, tricyclazole

F13) resistance induction, for example acibenzolar-S-methyl, probenazole, tiadinil, isotianil

F14) multisite, for example captafol, captan, chlorothalonil, copper salts such as: copper hydroxide, copper naphthenate, copper oxychloride, copper sulfate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulfur and sulfur preparations containing calcium polysulfide, thiram, tolylfluanid, zineb, ziram

F15) unknown mechanism, for example amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulfate, diphenylamine, ethaboxam, ferimzone, flumetover, flusulfamide, fluopicolid, fluoroimid, fosatyl-Al, hexachlorobenzene, 8-hydroxyquinoline sulfate, iprodione, irumamycin, isotianil, methasulfocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrolnitrin, quintozene, tecloftalam, tecnazene, triazoxide, trichlamide, zarilamid and 2,3,5,6-tetrachloro-4-(methylsulfonyl)pyridine, N-(4-chloro-2-nitrophenyl)-N-ethyl-4-methylbenzenesulfonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyI)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl]-.alpha.-(methoxymethylene)benzacetate, 4-chloro-alpha-propynyloxy-N-[2-[3-methoxy-4-(2-propynyloxy)phenyl]ethyl]benzacetamide, (2S)-N-[2-[4-[[3-(4-chlorophenyl)-2-propynyl]oxy]-3-methoxyphenyl]ethyl]-3-methyl-2-[(methylsulfonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine, N-[1-(5-bromo-3-chloropyridin-2-yl)ethyl]-2,4-dichloronicotinamide, N-(5-bromo-3-chloro-pyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3(1-fluoro-2-phenylethyl)oxy]phenyl]ethylidene]amino]oxy]-methyl]-alpha-(methoxyimino)-N-methyl-alphaE-benzacetamide, N-{2-[3-chloro-5-(tri-fluoromethyl)pyridin-2-yl]ethyl}-2-(trifluoromethyl)benzamide, N-(3′,4′-dichloro-5-fluoro-biphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide.

Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulfate and other copper preparations.

Insecticides/acaricides/nematicides:

I1) acetylcholine esterase (AChE) inhibitors, a) from the substance group of the carbamates, for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, fenoxycarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate, b) from the group of the organophosphates, for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion

I2) sodium channel modulators/voltage-dependent sodium channel blockers, a) from the group of the pyrethroids, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, eflusilanate, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, pyrethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum), b) DDT, c) oxadiazines, for example indoxacarb, d) semicarbazones, for example metaflumizone (BAS3201)

I3) acetylcholine receptor agonists/antagonists, a) from the group of the chloronicotinyls, for example acetamiprid, AKD 1022, clothianidin, dinotefuran, imidacloprid, imidaclothiz, nitenpyram, nithiazine, thiacloprid, thiamethoxam, b) nicotine, bensultap, cartap;

I4) acetylcholine receptor modulators from the group of the spinosyns, for example spinosad

I5) GABA-controlled chloride channel antagonists, a) from the group of the organochlorines, for example camphechlor, chlorodane, endosulfan, gamma-HCH, HCH, heptachlor, lindane, methoxychlor, b) fiproles, for example acetoprole, ethiprole, fipronil, pyrafluprole, pyriprole, vaniliprole;

I6) chloride channel activators, for example abamectin, emamectin, emamectin benzoate, ivermectin, lepimectin, milbemycin;

I7) juvenile hormone mimetics, for example diofenolan, epofenonane, fenoxycarb, hydroprene, kinoprene, methoprene, pyriproxifen, triprene;

I8) ecdysone agonists/disruptors, for example chromafenozide, halofenozide, methoxyfenozide, tebufenozide;

I9) chitin biosynthesis inhibitors, for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron, buprofezin, cyromazine;

I10) inhibitors of oxidative phosphorylation, a) ATP disruptors, for example diafenthiuron, b) organotin compounds, for example azocyclotin, cyhexatin, fenbutatin oxide;

I11) decouplers of oxidative phosphorylation by interruption of the H-proton gradient, a) from the group of the pyrroles, for example chlorofenapyr, b) from the class of the dinitrophenols, for example binapacyrl, dinobuton, dinocap, DNOC, meptyldinocap;

I12) site I electron transport inhibitors, for example METIs, especially, as examples, fenazaquin, fenpyroximate, pyrimidifen, pyridaben, tebufenpyrad, tolfenpyrad or else hydramethylnon, dicofol

I13) site II electron transport inhibitors, for example rotenone

I14) site III electron transport inhibitors, for example acequinocyl, fluacrypyrim

I15) microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins, for example Cry1Ab, Cry1Ac, Cry1Fa, Cry2Ab, mCry3A, Cry3Ab, Cry3Bb, Cry34/35Ab1

I16) lipid synthesis inhibitors, a) from the group of the tetronic acids, for example spirodiclofen, spiromesifen, b) from the class of the tetramic acids, for example spirotetramat, cis-3-(2,5-dimethylphenyl)-4-hydroxy-8-methoxy-1-azaspiro[4.5]dec-3-en-2-one

I17) octopaminergic agonists, for example amitraz

I18) inhibitors of magnesium-stimulated ATPase, for example propargite

I19) nereistoxin analogs, for example thiocyclam hydrogen oxalate, thiosultap-sodium

I20) ryanodine receptor agonists, a) from the group of the benzenedicarboxamides, for example flubendiamide, b) from the group of the anthranilamides, for example rynaxypyr(3-bromo-N-{4-chloro-2-methyl-6-[(methylamino)carbonyl]phenyl}-1-(3-chloropyridin-2-yl)-1H-pyrazole-5-carboxamide), cyazypyr (ISO-proposed) (3-bromo-N-{4-cyano-2-methyl-6-[(methylamino)carbonyl]phenyl}-1-(3-chloropyridin-2-yl)-1H-pyrazole-5-carboxamide) (known from WO 2004067528) and 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) or methyl 2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (known from WO2007/043677)

I21) biologics, hormones or pheromones, for example azadirachtin, Bacillus spec., Beauveria spec., codlemone, Metarrhizium spec., Paecilomyces spec., thuringiensin, Verticillium spec.

I22) active ingredients with unknown or nonspecific mechanisms of action, a) fumigants, for example aluminum phosphide, methyl bromide, sulfuryl fluoride, b) antifeedants, for example cryolite, flonicamide, pymetrozine, c) mite growth inhibitors, for example clofentezine, etoxazole, hexythiazox, d) amidoflumet, benclothiaz, benzoximate, bifenazate, bromopropylate, buprofezin, chinomethionat, chlorodimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin and the following known active compounds: 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-chloropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP0539588), 4-{[(6-chloropyrid-3-yl)methyl](methyl)amino}furan-2(5H)-one (known from EP0539588), [1-(6-chloropyridin-3-yl)ethyl](methyl)oxido-λ⁴-sulfanylidenecyanamide (known from WO 2007/149134) and the diastereomers thereof {[(1R)-1-(6-chloropyridin-3-yl)ethyl](methyl)oxido-lambda⁶-sulfanylidene}cyanamide and {[(1S)-1-(6-chloropyridin-3-yl)ethyl](methyl)oxido-lambda⁶-sulfanylidene}cyanamide (likewise known from WO 2007/149134) and sulfoxaflor (likewise known from WO 2007/149134), 1-[2-fluoro-4-methyl-5-[(2,2,2-trifluoroethyl)sulfinyl]phenyl]-3-(trifluoromethyl)-1H-1,2,4-triazol-5-amine (known from WO 2006/043635), [(3S,4aR,12R,12aS,12bS)-3-[(cyclopropylcarbonyl)oxy]-6,12-dihydroxy-4,12b-dimethyl-11-oxo-9-(pyridin-3-yl)-1,3,4,4a,5,6,6a,12,12a,12b-decahydro-2H,11H-benzo[f]pyrano[4,3-b]chromen-4-yl]methyl cyclopropane-carboxylate (known from WO 2006/129714), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluorometho)-N-ethyl-N-methyl-1,2-benzothiazole-3-amine 1,1-dioxide (known from WO2007/057407), N-[1-(2,3-dimethylphenyl)-2-(3,5-dimethylphenyl)ethyl]-4,5-dihydro-1,3-thiazole-2-amine (known from WO2008/104503), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO2003106457), 3-(2,5-dimethylphenyI)-4-hydroxy-8-methoxy-1,8-diazaspiro[4.5]dec-3-en-2-one (known from WO2009049851), 3-(2,5-dimethylphenyl)-8-methoxy-2-oxo-1,8-diazaspiro[4.5]dec-3-en-4-yl ethyl carbonate (known from WO2009049851), 4-(but-2-yn-1-yloxy)-6-(3,5-dimethylpiperidin-1-yl)-5-fluoropyrimidine (known from WO2004099160), (2,2,3,3,4,4,5,5-octafluoropentyl)(3,3,3-trifluoropropyl)malononitrile (known from WO2005063094), (2,2,3,3,4,4,5,5-octafluoropentyl)(3,3,4,4,4-pentafluorobutyl)malononitrile (known from WO2005063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO2007040280/282), 2-ethyl-7-methoxy-3-methyl-6-[(2,2,3,3-tetrafluoro-2,3-dihydro-1,4-benzodioxin-6-yl)oxy]quinolin-4-yl methyl carbonate (known from JP2008110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008110953), PF1364 (Chemical Abstracts No. 1204776-60-2, known from JP2010018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007075459), 5-[5-(2-chloropyridin-4-yl)-5-(trifluoromethyl)-4,5-dihydro-1,2-oxazol-3-yl]-2-(1H-1,2,4-1-yl)benzonitrile (known from WO2007075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005085216).

Safeners are preferably selected from the group consisting of:

S1) compounds of the formula (S1)

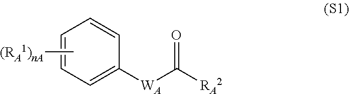

where the symbols and indices are each defined as follows:
n_{A} is a natural number from 0 to 5, preferably 0 to 3;
R_{A}¹ is halogen, (C₁-C₄)-alkyl, (C₁-C₄)-alkoxy, nitro or (C₁-C₄)-haloalkyl;

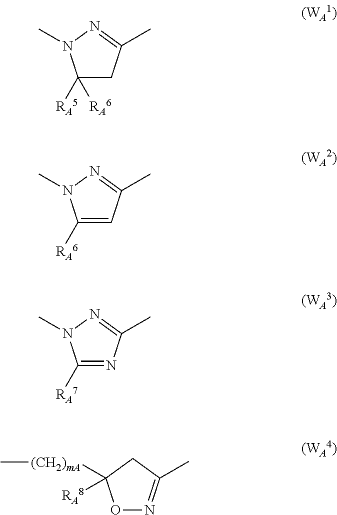

W_{A} is an unsubstituted or substituted divalent heterocyclic radical from the group consisting of partially unsaturated or aromatic five-membered heterocycles having 1 to 3 hetero ring atoms from the group of N and O, where at least one nitrogen atom and at most one oxygen atom is present in the ring, preferably a radical from the group consisting of (W_{A}¹) to (W_{A}⁴),
m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S1) and which is unsubstituted or substituted by radicals from the group consisting of (C₁-C₄)-alkyl, (C₁-C₄)-alkoxy and optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbyl radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹ where R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are the same or different and are each hydrogen, (C₁-C₈)-alkyl, (C₁-C₈)-haloalkyl, (C₃-C₁₂)-cycloalkyl or substituted or unsubstituted phenyl;
   preferably:
a) compounds of the type of the dichlorophenylpyrazoline-3-carboxylic acid (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds, as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds, as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds, as described, for example, in EP-A-268554;
d) compounds of the triazolecarboxylic acid type (S1^{d}), preferably compounds such as fenchlorazole(-ethyl), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.

S2) Quinoline derivatives of the formula (S2),

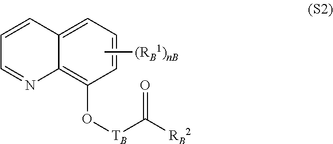

where the symbols and indices are each defined as follows:
R_{B}¹ is halogen, (C₁-C₄)-alkyl, (C₁-C₄)-alkoxy, nitro or (C₁-C₄)-haloalkyl;
n_{B} is a natural number from 0 to 5, preferably 0 to 3;
R_{B}² is OR_{B}³, SR_{B}³ or NR_{B}³R_{B}⁴ or a saturated
   or unsaturated 3- to 7-membered heterocycle having at least one nitrogen atom and up to 3 heteroatoms, preferably from the group consisting of O and S, which is attached via the nitrogen atom to the carbonyl group in (S2) and which is unsubstituted or substituted by radicals from the group consisting of (C₁-C₄)-alkyl, (C₁-C₄)-alkoxy and optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbyl radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁- or C₂)-alkanediyl chain which is unsubstituted or substituted by one or two (C₁-C₄)-alkyl radicals or by [(C₁-C₃)-alkoxy]carbonyl;
   preferably:
a) compounds of the 8-quinolinoxyacetic acid type (S2^{a}), preferably 1-methylhexyl(5-chloro-8-quinolinoxy)acetate (“cloquintocet-mexyl”) (S2-1), 1,3-dimethylbut-1-yl(5-chloro-8-quinolinoxy)acetate (S2-2), 4-allyloxybutyl(5-chloro-8-quinolinoxy)acetate (S2-3), 1-allyloxyprop-2-yl(5-chloro-8-quinolinoxy)acetate (S2-4), ethyl(5-chloro-8-quinolinoxy)acetate (S2-5), methyl(5-chloro-8-quinolinoxy)acetate (S2-6), allyl(5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S2^{b}), preferably compounds such as diethyl(5-chloro-8-quinolinoxy)malonate, diallyl(5-chloro-8-quinolinoxy)malonate, methyl ethyl(5-chloro-8-quinolinoxy)malonate and related compounds as described in EP-A-0 582 198.
S3) Compounds of the formula (S3)

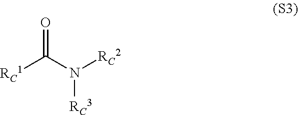

where the symbols and indices are each defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are the same or different and are each hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring; preferably: active ingredients of the dichloroacetamide type, which are frequently used as pre-emergence safeners (soil-acting safeners), for example “dichlormid” (N,N-diallyl-2,2-dichloroacetamide) (S3-1), “R-29148” (3-dichloroacetyl-2,2,5-trimethyl-1,3-oxazolidine) from Stauffer (S3-2), “R-28725” (3-dichloroacetyl-2,2-dimethyl-1,3-oxazolidine) from Stauffer (S3-3), “benoxacor” (4-d ich loroacetyl-3,4-d ihyd ro-3-methyl-2H-1,4-benzoxazine) (S3-4), “PPG-1292” (N-allyl-N-[(1,3-dioxolan-2-yl)methyl]dichloroacetamide) from PPG Industries (S3-5), “DKA-24” (N-allyl-N-[(allylaminocarbonyl)methyl]dichloroacetamide) from Sagro-Chem (S3-6), “AD-67” or “MON 4660” (3-dichloroacetyl-1-oxa-3-azaspiro[4,5]decane) from Nitrokemia or Monsanto (S3-7), “TI-35” (1-dichloroacetylazepane) from TRI-Chemical RT (S3-8), “diclonon” (dicyclonone) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10); and the (R) isomer thereof (S3-11).
S4) N-Acylsulfonamides of the formula (S4) and salts thereof

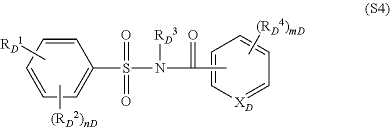

where the symbols and indices are each defined as follows:
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group of nitrogen, oxygen and sulfur, where the seven latter radicals are substituted by v_{D} substituents from the group of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl, and in the case of cyclic radicals also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxy, (C₁-C₄)-alkyl, (C₁-C₄)-alkoxy and (C₁-C₄)-alkylthio, or
R_{D}⁵ and R_{D}⁶ together with the nitrogen atom bearing them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group of halogen, (C₁-C₄)-alkoxy, (C₁-C₆)-haloalkoxy and (C₁-C₄)-alkylthio, and in the case of cyclic radicals also (C₁-C₄)-alkyl and (C₁-C₄)-haloalkyl;
n_{D} is 0, 1 or 2;
m_{D} is 1 or 2;
v_{D} is 0, 1, 2 or 3;
   among these, preference is given to compounds of the N-acylsulfonamide type, for example of the formula (S4^{a}) below, which are known, for example, from WO-A-97/45016

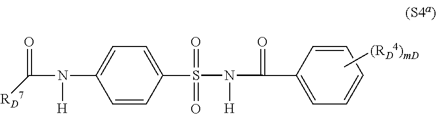

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃,
m_{D} is 1 or 2;
v_{D} is 0, 1, 2 or 3;
   and also to acylsulfamoylbenzamides, for example of the formula (S4^{b}) below, which are known, for example, from WO-A-99/16744,

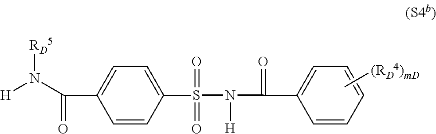

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5);
   and to compounds of the N-acylsulfamoylphenylurea type, of the formula (Se), which are known, for example, from EP-A-365484,

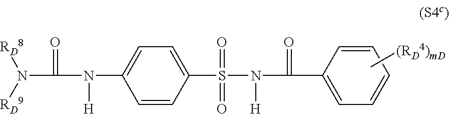

in which
R_{D}⁸ and R_{D}⁹ are each independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃,
m_{D} is 1 or 2;
   for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
S5) Active ingredients from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

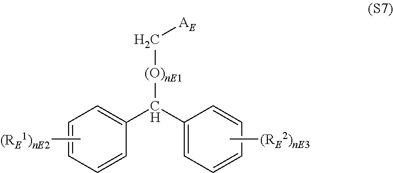

where the symbols and indices are each defined as follows:
R_{E}¹, R_{E}² are each independently halogen, (C₁-C₄)alkyl, (C₁-C₄)alkoxy, (C₁-C₄)haloalkyl, (C₁-C₄)alkylamino, di-(C₁-C₄)alkylamino, nitro;
A_{E} is COOR_{E}³ or COSR_{E}⁴
R_{E}³, R_{E}⁴ are each independently hydrogen, (C₁-C₄)alkyl, (C₂-C₆)alkenyl, (C₂-C₄)alkynyl, cyanoalkyl, (C₁-C₄)haloalkyl, phenyl, nitrophenyl, benzyl, halobenzyl, pyridinylalkyl and alkylammonium,
n_{E}¹ is 0 or 1;
n_{E}², n_{E}³ are each independently 0, 1 or 2,
   preferably diphenylmethoxyacetic acid, ethyl diphenylmethoxyacetate, methyl diphenylmethoxyacetate (CAS reg. no. 41858-19-9) (S7-1).
S8) Compounds of the formula (S8), as described in WO-A-98/27049,

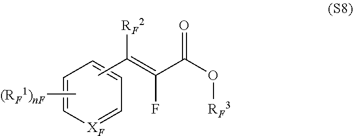

in which
X_{F} is CH or N,
n_{F} if X_{F}═N is an integer from 0 to 4 and
if X_{F}═CH is an integer from 0 to 5,
R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the carbon-containing radicals mentioned above is unsubstituted or substituted by one or more, preferably by up to three, identical or different radicals from the group consisting of halogen and alkoxy; or salts thereof,
   preferably compounds in which
X_{F} is CH,
n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably by up to three, identical or different radicals from the group consisting of halogen and alkoxy; or salts thereof.
S9) Active ingredients from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no.: 219479-18-2), 1,2-dihydro-4-hydroxy-1-methyl-3-(5-tetrazolylcarbonyl)-2-quinolone (CAS reg. no.: 95855-00-8), as described in WO-A-1999/000020.
S10) Compounds of the formula (S10^{a}) or (S10^{b}) as described in WO-A-2007/023719 and WO-A-2007/023764

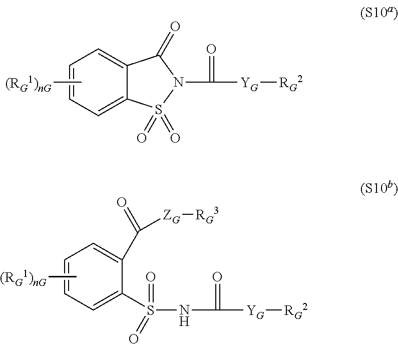

in which
R_{G}¹ is halogen, (C₁-C₄)-alkyl, methoxy, nitro, cyano, CF₃, OCF₃
Y_{G}, Z_{G}are each independently O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active ingredients of the oxyimino compound type (S11), which are known as seed-dressing compositions, for example “oxabetrinil” ((Z)-1,3-dioxolan-2-yl-methoxyimino(phenyl)acetonitrile) (S11-1), which is known as a seed-dressing safener for millet against damage by metolachlor, “fluxofenim” (1-(4-chlorophenyl)-2,2,2-trifluoro-1-ethanone O-(1,3-dioxolan-2-ylmethyl)oxime) (S11-2), which is known as a seed-dressing safener for millet against damage by metolachlor, and “cyometrinil” or “CGA-43089” ((Z)-cyanomethoxy-imino(phenyl)acetonitrile) (S11-3), which is known as a seed-dressing safener for millet against damage by metolachlor.
S12) Active ingredients from the class of the isothiochromanones (S12), for example methyl [(3-oxo-1H-2-benzothiopyran-4(3H)-ylidene)methoxy]acetate (CAS reg. no.: 205121-04-6) (S12-1) and related compounds from WO-A-1998/13361.
S13) One or more compounds from group (S13): “naphthalic anhydride” (1,8-naphthalenedicarboxylic anhydride) (S13-1), which is known as a seed-dressing safener for corn against damage by thiocarbamate herbicides, “fenclorim” (4,6-dichloro-2-phenylpyrimidine) (S13-2), which is known as a safener for pretilachlor in sown rice, “flurazole” (benzyl 2-chloro-4-trifluoromethyl-1,3-thiazole-5-carboxylate) (S13-3), which is known as a seed-dressing safener for millet against damage by alachlor and metolachlor, “CL 304415” (CAS reg. no. 31541-57-8) (4-carboxy-3,4-dihydro-2H-1-benzopyran-4-acetic acid) (S13-4) from American Cyanamid, which is known as a safener for corn against damage by imidazolinones, “MG 191” (CAS reg. no. 96420-72-3) (2-dichloromethyl-2-methyl-1,3-dioxolane) (S13-5) from Nitrokemia, which is known as a safener for corn, “MG-838” (CAS reg. no. 133993-74-5) (2-propenyl 1-oxa-4-azaspiro[4.5]decane-4-carbodithioate) (S13-6) from Nitrokemia, “disulfoton” (O,O-diethyl S-2-ethylthioethyl phosphorodithioate) (S13-7), “dietholate” (O,O-diethyl O-phenylphosphorothioate) (S13-8), “mephenate” (4-chlorophenyl methyl carbamate) (S13-9).
S14) Active ingredients which, in addition to herbicidal action against harmful plants, also have safener action on crop plants such as rice, for example “dimepiperate” or “MY-93” (S-1-methyl-1-phenylethylpiperidine-1-carbothioate), which is known as a safener for rice against damage by the herbicide molinate, “daimuron” or “SK 23” (1-(1-methyl-1-phenylethyl)-3-p-tolylurea), which is known as a safener for rice against damage by the herbicide imazosulfuron, “cumyluron”=“JC-940” (3-(2-chlorophenylmethyl)-1-(1-methyl-1-phenylethyl)urea, see JP-A-60087254), which is known as a safener for rice against damage by some herbicides, “methoxyphenon” or “NK 049” (3,3′-dimethyl-4-methoxybenzophenone), which is known as a safener for rice against damage by some herbicides, “CSB” (1-bromo-4-(chloromethylsulfonyl)benzene) from Kumiai, (CAS reg. no. 54091-06-4), which is known as a safener against damage by some herbicides in rice.
S15) Compounds of the formula (S15) or tautomers thereof as described in WO-A-2008/131861 and WO-A-2008/131860

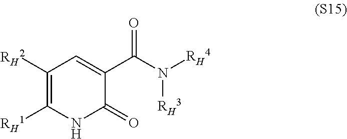

in which
R_{H}¹ is a (C₁-C₆)haloalkyl radical and
R_{H}² is hydrogen or halogen and
R_{H}³, R_{H}⁴ are each independently hydrogen, (C₁-C₁₆)alkyl, (C₂-C₁₆)alkenyl or (C₂-C₁₆)alkynyl, where each of the 3 latter radicals is unsubstituted or substituted by one or more radicals from the group of halogen, hydroxyl, cyano, (C₁-C₄)alkoxy, (C₁-C₄)haloalkoxy, (C₁-C₄)alkylthio, (C₁-C₄)alkylamino, di[(C₁-C₄)alkyl]amino, [(C₁-C₄)alkoxy]carbonyl, [(C₁-C₄)haloalkoxy]carbonyl, (C₃-C₆)cycloalkyl which is unsubstituted or substituted, phenyl which is unsubstituted or substituted, and heterocyclyl which is unsubstituted or substituted, or (C₃-C₆)cycloalkyl, (C₄-C₆)cycloalkenyl, (C₃-C₆)cycloalkyl which is fused on one side of the ring to a 4- to 6-membered saturated or unsaturated carbocyclic ring, or (C₄-C₆)cycloalkenyl which is fused on one side of the ring to a 4- to 6-membered saturated or unsaturated carbocyclic ring, where each of the 4 latter radicals is unsubstituted or substituted by one or more radicals from the group of halogen, hydroxyl, cyano, (C₁-C₄)alkyl, (C₁-C₄)haloalkyl, (C₁-C₄)alkoxy, (C₁-C₄)haloalkoxy, (C₁-C₄)alkylthio, (C₁-C₄)alkylamino, di[(C₁-C₄)alkyl]amino, [(C₁-C₄)alkoxy]carbonyl, [(C₁-C₄)haloalkoxy]carbonyl, (C₃-C₆)cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)alkenyloxy, (C₂-C₆)alkynyloxy or (C₂-C₄)haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, in addition to the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)alkyl, (C₁-C₄)haloalkyl, (C₁-C₄)alkoxy, (C₁-C₄)haloalkoxy and (C₁-C₄)alkylthio.
S16) Active ingredients which are used primarily as herbicides but also have safener action on crop plants, for example (2,4-dichlorophenoxy)acetic acid (2,4-D), (4-chlorophenoxy)acetic acid, (R,S)-2-(4-chloro-o-tolyloxy)propionic acid (mecoprop), 4-(2,4-dichlorophenoxy)butyric acid (2,4-DB), (4-chloro-o-tolyloxy)acetic acid (MCPA), 4-(4-chloro-o-tolyloxy)butyric acid, 4-(4-chlorophenoxy)butyric acid, 3,6-dichloro-2-methoxybenzoic acid (dicamba), 1-(ethoxycarbonyl)ethyl 3,6-dichloro-2-methoxybenzoate (lactidichlor-ethyl).

Substances which Influence Plant Maturity:

Usable combination partners for the inventive compounds in mixture formulations or in a tankmix are, for example, known active ingredients based on inhibition of, for example, 1-aminocyclopropane-1-carboxylate synthase, 1-aminocyclopropane-1-carboxylate oxidase and the ethylene receptors, e.g. ETR1, ETR2, ERS1, ERS2 or EIN4, as described, for example, in Biotechn. Adv. 2006, 24, 357-367; Bot. Bull. Acad. Sin. 199, 40, 1-7 or Plant Growth Reg. 1993, 13, 41-46 and literature cited therein.

Examples of known substances which influence plant maturity and can be combined with the inventive compounds include the active ingredients which follow (the compounds are designated by the “common name” according to the International Organization for Standardization (ISO) or by the chemical name or by the code number) and always encompass all use forms, such as acids, salts, esters and isomers, such as stereoisomers and optical isomers. By way of example, one use form and in some cases a plurality of use forms are mentioned:

rhizobitoxine, 2-aminoethoxyvinylglycine (AVG), methoxyvinylglycine (MVG), vinylglycine, aminooxyacetic acid, sinefungin, S-adenosylhomocysteine, 2-keto-4-methyl thiobutyrate, 2-(methoxy)-2-oxoethyl(isopropylidene)aminooxyacetate, 2-(hexyloxy)-2-oxoethyl(isopropylidene)aminooxyacetate, 2-(isopropyloxy)-2-oxoethyl(cyclohexylidene)aminooxyacetate, putrescine, spermidine, spermine, 1,8-diamino-4-aminoethyloctane, L-canaline, daminozide, methyl 1-aminocyclopropyl-1-carboxylate, N-methyl-1-aminocyclopropyl-1-carboxylic acid, 1-aminocyclopropyl-1-carboxamide, substituted 1-aminocyclopropyl-1-carboxylic acid derivatives as described in DE3335514, EP30287, DE2906507 or U.S. Pat. No. 5123951, 1-aminocyclopropyl-1-hydroxamic acid, 1-methylcyclopropene, 3-methylcyclopropene, 1-ethylcyclopropene, 1-n-propylcyclopropene, 1-cyclopropenylmethanol, carvone, eugenol

Herbicides or Plant Growth Regulators:

Usable combination partners for the inventive compounds in mixture formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, gibberellin biosynthesis, as described, for example, in Weed Research 26 (1986) 441-445 or “The Pesticide Manual”, 14th edition, The British Crop Protection Council and the Royal Soc. of Chemistry, 2006 and literature cited therein.

Examples of known herbicides or plant growth regulators which can be combined with the inventive compounds include the active ingredients which follow (the compounds are designated by the “common name” according to the International Organization for Standardization (ISO) or by the chemical name or by the code number) and always encompass all use forms, such as acids, salts, esters and isomers, such as stereoisomers and optical isomers. By way of example, one use form and in some cases a plurality of use forms are mentioned:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlornitrofen, chlorophthalim, chlorthal-dimethyl, chlortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. O-(2,4-dimethyl-6-nitrophenyl) O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazine-3-amine, MT-5950, i.e. N-[3-chloro-4-(1-methylethyl)phenyI]-2-methylpentanamide, NGGC-011, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate(pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), sulfentrazone, sulfometuron, sulfometuron-methyl, sulfosate(glyphosate-trimesium), sulfosulfuron, SYN-523, SYP-249, i.e. 1-ethoxy-3-methyl-1-oxobut-3-en-2-yl5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryne, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

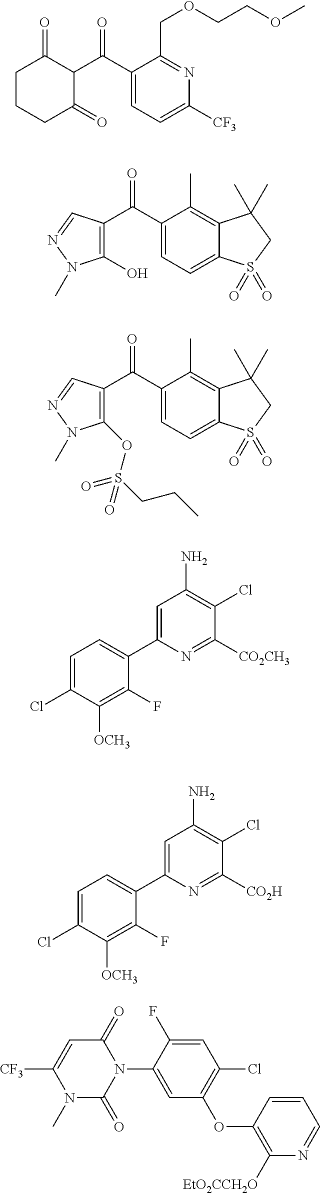

The invention is to be illustrated by the biological examples which follow, but without restricting it thereto.

Biological Examples:

Seeds of monocotyledonous and dicotyledonous crop plants were placed in sandy loam in wood-fiber pots, covered with soil and cultivated in a greenhouse under good growth conditions. The test plants were treated at the early leaf stage (BBCH10-BBCH13). To ensure uniform water supply before commencement of stress, the potted plants were supplied with the maximum amount of water immediately beforehand by dam irrigation and, after application, transferred in plastic inserts in order to prevent subsequent, excessively rapid drying. The inventive compounds, formulated in the form of wettable powders (WP), wettable granules (WG), suspension concentrates (SC) or emulsion concentrates (EC), were sprayed onto the green parts of the plants as an aqueous suspension at an equivalent water application rate of 600 l/ha with addition of 0.2% wetting agent (agrotin). Substance application was followed immediately by stress treatment of the plants (cold or drought stress). For cold stress treatment, the plants were kept under the following controlled conditions:

“day”: 12 hours with illumination at 8° C.

“night”: 12 hours without illumination at 1° C.

Drought stress was induced by gradual drying out under the following conditions:

“day”: 14 hours with illumination at 26° C.

“night”: 10 hours without illumination at 18° C.

The duration of the respective stress phases was guided mainly by the state of the untreated, stressed control plants and thus varies from crop to crop. It was ended (by re-irrigating or transfer to a greenhouse with good growth conditions) as soon as irreversible damage is observed on the untreated, stressed control plants. In the case of dicotyledonous crops, for example oilseed rape and soya, the duration of the drought stress phase was between 3 and 5 days, in the case of monocotyledonous crops, for example wheat, barley or corn, between 6 and 10 days. The duration of the cold stress phase varied between 12 and 14 days.

The end of the stress phase was followed by an approx. 5-7-day recovery phase, during which the plants were once again kept under good growth conditions in a greenhouse. In order to rule out any influence of the effects observed by any fungicidal action of the test compounds, it was additionally ensured that the tests proceed without fungal infection and without infection pressure.

After the recovery phase had ended, the intensities of damage were rated visually compared to untreated, unstressed controls of the same age (in the case of drought stress) or the same growth stage (in the case of cold stress). The intensity of damage was first assessed as a percentage (100%=plants have died, 0%=like control plants). These values were then used to calculate the efficacy of the test compounds (=percentage reduction in the intensity of damage as a result of substance application) by the following formula:

$$EF = \frac{( {DV}_{us} - {DV}_{ts} ) \times 100}{{DV}_{us}}$$

EF: efficacy (%)

DVᵤₛ: damage value of the untreated, stressed control

DVₜₛ: damage value of the plants treated with test compound

The tables below list mean values in each case from three results of the same test.

Effects of selected compounds of the formula (I) under cold stress:

| | | | | EF |
|---|---|---|---|---|
| No. | Substance | Dosage | Unit | (ZEAMX) |
| 1 | 1-2 | 5 | g/ha | >5 |
| 2 | 1-14 | 25 | g/ha | >5 |
| 3 | 1-17 | 25 | g/ha | >5 |

Effects of selected compounds of the formula (I) under drought stress:

| | | | | EF |
|---|---|---|---|---|
| No. | Substance | Dosage | Unit | (HORVS) |
| 1 | 1-1 | 100 | g/ha | >5 |
| 2 | 1-2 | 25 | g/ha | >5 |
| 3 | 1-8 | 50 | g/ha | >5 |
| 4 | 1-17 | 25 | g/ha | >5 |
| 5 | 1-21 | 25 | g/ha | >5 |
| 6 | 1-24 | 25 | g/ha | >5 |
| 7 | 1-27 | 100 | g/ha | >5 |
| 8 | 2-7 | 25 | g/ha | >5 |
| 9 | 4-7 | 25 | g/ha | >5 |

| | | | | EF |
|---|---|---|---|---|
| No. | Substance | Dosage | Unit | (BRSNS) |
| 1 | 1-2 | 25 | g/ha | >5 |
| 2 | 1-5 | 25 | g/ha | >5 |
| 3 | 1-6 | 25 | g/ha | >5 |
| 4 | 1-8 | 50 | g/ha | >5 |
| 5 | 1-14 | 50 | g/ha | >5 |
| 6 | 1-17 | 50 | g/ha | >5 |
| 7 | 1-24 | 25 | g/ha | >5 |
| 8 | 1-50 | 25 | g/ha | >5 |
| 9 | 1-52 | 25 | g/ha | >5 |
| 10 | 1-69 | 25 | g/ha | >5 |
| 11 | 1-75 | 25 | g/ha | >5 |
| 12 | 1-89 | 250 | g/ha | >5 |
| 13 | 2-7 | 25 | g/ha | >5 |
| 14 | 4-7 | 25 | g/ha | >5 |
| 15 | 5-8 | 250 | g/ha | >5 |

| | | | | EF |
|---|---|---|---|---|
| No. | Substance | Dosage | Unit | (ZEAMX) |
| 1 | 1-2 | 25 | g/ha | >5 |
| 2 | 1-5 | 50 | g/ha | >5 |
| 3 | 1-6 | 25 | g/ha | >5 |
| 4 | 1-8 | 25 | g/ha | >5 |
| 5 | 1-14 | 25 | g/ha | >5 |
| 6 | 1-17 | 25 | g/ha | >5 |
| 7 | 1-50 | 250 | g/ha | >5 |
| 8 | 1-52 | 25 | g/ha | >5 |
| 9 | 1-84 | 25 | g/ha | >5 |
| 10 | 1-89 | 250 | g/ha | >5 |
| 11 | 1-90 | 250 | g/ha | >5 |

| | | | | EF |
|---|---|---|---|---|
| No. | Substance | Dosage | Unit | (TRZAS) |
| 1 | 1-2 | 25 | g/ha | >5 |
| 2 | 1-5 | 250 | g/ha | >5 |
| 3 | 1-6 | 25 | g/ha | >5 |
| 4 | 1-17 | 250 | g/ha | >5 |
| 5 | 1-24 | 25 | g/ha | >5 |
| 6 | 1-50 | 250 | g/ha | >5 |
| 7 | 1-52 | 25 | g/ha | >5 |
| 8 | 1-69 | 25 | g/ha | >5 |
| 9 | 1-90 | 250 | g/ha | >5 |
| 10 | 2-7 | 25 | g/ha | >5 |
| 11 | 4-7 | 25 | g/ha | >5 |
| 12 | 5-8 | 25 | g/ha | >5 |

In the above tables:

BRSNS=Brassica napus

HORVS=Hordeum vulgare

TRZAS=Triticum aestivum

ZEAMX=Zea mays

Similar results were also achieved with further compounds of the formula (I), also in the case of application to different plant species.

1. A method of increasing tolerance to abiotic stress in a plant comprising applying a fluoroalkyl-substituted 2-amidobenzimidazole of formula (I), or a salt thereof, 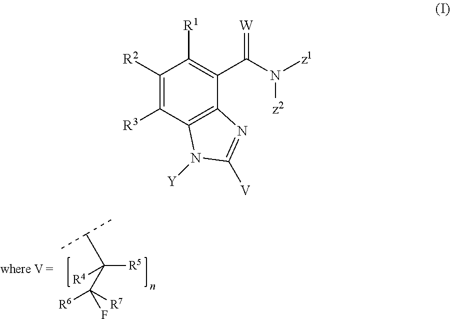
where
R¹, R², R³ are each independently H, halogen, branched or unbranched alkyl, cycloalkyl, alkenyl, alkynyl, aryl, arylalkyl, heteroaryl, alkoxyalkyl, alkylthio, fluoroalkylthio, haloalkyl, alkoxy, haloalkoxy;
R⁴ is H, unbranched alkyl, halogen, haloalkyl, branched alkyl, unbranched alkenyl, branched alkenyl, cycloalkyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁵ is H, unbranched alkyl, halogen, haloalkyl, branched alkyl, cycloalkyl, cycloalkenyl, alkoxy, haloalkoxy;
R⁶ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁷ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
and
n is 0, 1, 2, 3, 4, 5, 6;
W is oxygen, sulfur;
Y is H, unbranched or branched alkyl, cycloalkyl, cycloalkylalkyl, cycloalkenyl, cyanoalkyl, unbranched or branched alkenylalkyl, branched or unbranched haloalkyl, alkynylalkyl, arylalkyl, heteroarylalkyl, alkylcarbonyl, cycloalkylcarbonyl, arylcarbonyl, arylalkylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, alkoxyalkyl;
Z¹ is H, unbranched or branched alkyl, cycloalkyl, halogen, unbranched or branched alkylalkenyl, branched or unbranched haloalkyl, alkynyl, alkenyl, cyanoalkyl, arylalkyl, heteroarylalkyl, alkylcarbonyl, alkoxycarbonyl, alkylsulfonyl, arylsulfonyl, cycloalkylsulfonyl, alkylsulfinyl, arylsulfinyl, cycloalkylsulfinyl, alkoxycarbonylalkyl;
and
Z² is H, unbranched or branched alkyl, cycloalkyl, unbranched or branched alkylalkenyl, branched or unbranched haloalkyl, alkynyl, alkenyl, cyanoalkyl, arylalkyl, heteroarylalkyl, alkylcarbonyl, alkoxycarbonyl;
or Z¹ and Z² together are part of an optionally substituted sulfilimine or amidine group or form an iminophosphorane.

2. The method as claimed in claim 1, where, in formula (I),
R¹, R², R³ are each independently H, fluorine, chlorine, bromine, iodine, branched or unbranched (C₁-C₆)-alkyl, (C₃-C₇)-cycloalkyl, unbranched (C₂-C₆)-alkenyl, branched (C₃-C₆)-alkenyl, (C₂-C₆)-alkynyl, aryl, heteroaryl, aryl-(C₁-C₆)-alkyl, (C₁-C₆)-alkoxy-(C₁-C₆)-alkyl, (C₁-C₆)-alkylthio, (C₁-C₆)-fluoroalkylthio, (C₁-C₆)-haloalkyl, (C₁-C₆)-alkoxy, (C₁-C₆)-haloalkoxy;
R⁴ is H, unbranched (C₁-C₆)-alkyl, fluorine, chlorine, bromine, iodine, (C₁-C₆)-haloalkyl, branched (C₃-C₆)-alkyl, unbranched (C₂-C₆)-alkenyl, branched (C₃-C₆)-alkenyl, (C₃-C₇)-cycloalkyl, (C₁-C₈)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino-(C₁-C₆)-alkoxy;
R⁵ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇-cycloalkenyl;
R⁶ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl, (C₁-C₆)-alkoxy, (C₁-C₆)-alkylthio, (C₁-C₆)-haloalkoxy, (C₃-C₇)-cycloalkyl-(C₁-C₆)-alkoxy, (C₂-C₆)-alkynyl-(C₁-C₆)-alkoxy, (C₂-C₆)-alkenyl-(C₁-C₆)-alkoxy, (C₂-C₆)-alkenyloxy-(C₁-C₆)-alkoxy, (C₁-C₆)-alkyloxy-(C₁-C₆)-alkoxy, (C₁-C₆)-alkylamino-(C₁-C₆)-alkoxy, (C₁-C₆)-dialkylamino-(C₁-C₆)-alkoxy, (C₃-C₇)-cycloalkylamino-(C₁-C₆)-alkoxy;
R⁷ is H, unbranched (C₁-C₆)-alkyl, fluorine, chlorine, bromine, iodine, (C₁-C₆)-haloalkyl, branched (C₃-C₆)-alkyl, (C₃-C₇)-cycloalkyl, (C₃-C₇)-cycloalkenyl
n is 0, 1, 2, 3, 4, 5;
W is oxygen, sulfur;
Y is H, unbranched or branched (C₁-C₈)-alkyl, (C₃-C₇)-cycloalkyl, (C₃-C₇)-cycloalkyl-(C₁-C₆)-alkyl, (C₁-C₈)-cyanoalkyl, (C₃-C₇)-cycloalkenyl, unbranched or branched (C₂-C₆)-alkenyl-(C₁-C₆)-alkyl, (C₁-C₆)-haloalkyl, (C₂-C₆)-alkynyl-(C₁-C₆)-alkyl, aryl-(C₁-C₆)-alkyl, heteroaryl-(C₁-C₆)-alkyl, (C₁-C₆)-alkylcarbonyl, (C₃-C₇)-cycloalkylcarbonyl, arylcarbonyl, aryl-(C₁-C₆)-alkylcarbonyl, (C₁-C₆)-alkoxycarbonyl, (C₁-C₆)-alkylsulfonyl, (C₃-C₇)-cycloalkylsulfonyl, arylsulfonyl, heteroarylsulfonyl, (C₁-C₆)-al koxycarbonylcarbonyl, aryl-(C₁-C₆)-alkoxycarbonylcarbonyl, (C₁-C₆)-alkylaminothiocarbonyl, (C₁-C₆)-alkylaminocarbonyl, (C₁-C₆)-alkoxy-(C₁-C₆)-alkyl;
Z¹ is H, (C₁-C₈-alkyl, (C₃-C₇)-cycloalkyl, chlorine, bromine, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl, (C₁-C₆)-alkylsulfonyl, arylsulfonyl, (C₃-C₇)-cycloalkylsulfonyl, (C₁-C₆)-alkylsulfinyl, arylsulfinyl, (C₃-C₇)-cycloalkylsulfinyl, (C₁-C₆)-alkoxycarbonyl-(C₁-C₆)-alkyl;
and
Z² is H, (C₁-C₈)-alkyl, (C₃-C₇)-cycloalkyl, (C₂-C₆)-alkenyl-(C₁-C₆)-alkyl, (C₂-C₆)-alkynyl, (C₂-C₆)-alkenyl, (C₁-C₈)-haloalkyl, (C₁-C₈)-cyanoalkyl, heteroaryl-(C₁-C₈)-alkyl, aryl-(C₁-C₈)-alkyl, (C₁-C₆)-alkylcarbonyl, (C₁-C₆)-alkoxycarbonyl;
or Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N-((C₁-C₆)-alkyl-(C₃-C₇)-cycloalkyl)sulfanylidene group or an N,N-di-(C₁-C₆)-alkylformylidene group.

3. The method as claimed in claim 1, where, in formula (I), R¹, R², R³ are each independently H, fluorine, chlorine, bromine, iodine, branched or unbranched (C₁-C₄)-alkyl, (C₃-C₆)-cycloalkyl, (C₂-C₄)-alkenyl, (C₂-C₄)-alkynyl, aryl, heteroaryl, aryl-(C₁-C₄)-alkyl, (C₁-C₄)-alkoxy-(C₁-C₄)-alkyl, (C₁-C₄)-alkylthio, (C₁-C₄)-fluoroalkylthio, (C₁-C₄)-haloalkyl, (C₁-C₄)-alkoxy, (C₁-C₄)-haloalkoxy;
R⁴ is H, unbranched (C₁-C₄)-alkyl, fluorine, chlorine, bromine, (C₁-C₄)-haloalkyl, branched (C₃-C₆)-alkyl, unbranched (C₂-C₄)-alkenyl, branched (C₃-C₆)-alkenyl, (C₃-C₆)-cycloalkyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino-(C₁-C₄)-alkoxy;
R⁵ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl;
R⁶ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl, (C₁-C₈)-alkoxy, (C₁-C₄)-alkylthio, (C₁-C₆)-haloalkoxy, (C₃-C₆)-cycloalkyl-(C₁-C₄)-alkoxy, (C₂-C₆)-alkynyl-(C₁-C₄)-alkoxy, (C₂-C₆)-alkenyl-(C₁-C₄)-alkoxy, (C₂-C₆)-alkenyloxy-(C₁-C₄)-alkoxy, (C₁-C₆)-alkyloxy-(C₁-C₄)-alkoxy, (C₁-C₆)-alkylamino-(C₁-C₄)-alkoxy, (C₁-C₆)-dialkylamino-(C₁-C₄)-alkoxy, (C₃-C₆)-cycloalkylamino-(C₁-C₄)-alkoxy;
R⁷ is H, unbranched (C₁-C₄)-alkyl, fluorine, chlorine, bromine, (C₁-C₄)-haloalkyl, branched (C₃-C₆)-alkyl, (C₃-C₆)-cycloalkyl, (C₃-C₆)-cycloalkenyl
n is 0, 1, 2, 3, 4;
W is oxygen, sulfur;
Y is H, unbranched or branched (C₁-C₆)-alkyl, (C₃-C₆)-cycloalkyl, (C₃-C₆)-cycloalkyl-(C₁-C₄)-alkyl, (C₃-C₆)-cycloalkenyl, (C₁-C₄)-cyanoalkyl, unbranched or branched (C₂-C₄)-alkenyl-(C₁-C₄)-alkyl, (C₁-C₄)-haloalkyl, (C₂-C₄)-alkynyl-(C₁-C₄)-alkyl, aryl-(C₁-C₄)-alkyl, heteroaryl-(C₁-C₄)-alkyl, (C₁-C₅)-alkylcarbonyl, (C₃-C₆)-cycloalkylcarbonyl, arylcarbonyl, aryl-(C₁-C₄)-alkylcarbonyl, (C₁-C₄)-alkoxycarbonyl, (C₁-C₄)-alkylsulfonyl, (C₃-C₆)-cycloalkylsulfonyl, arylsulfonyl, heteroarylsulfonyl, (C₁-C₄)-al koxycarbonylcarbonyl, aryl-(C₁-C₄)-alkoxycarbonylcarbonyl, (C₁-C₄)-alkylaminothiocarbonyl, (C₁-C₄)-alkylaminocarbonyl, (C₁-C₄)-alkoxy-(C₁-C₄)-alkyl;
Z¹ is H, (C₁-C₆)-alkyl, (C₃-C₆)-cycloalkyl, chlorine, bromine, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl, (C₁-C₄)-alkylsulfonyl, arylsulfonyl, (C₃-C₆)-cycloalkylsulfonyl, (C₁-C₄)-alkylsulfinyl, arylsulfinyl, (C₃-C₆)-cycloalkylsulfinyl, (C₁-C₄)-alkoxycarbonyl-(C₁-C₄)-alkyl;
and
Z² is H, (C₁-C₆)-alkyl, (C₃-C₆)-cycloalkyl, (C₂-C₆)-alkenyl-(C₁-C₄)-alkyl, (C₂-C₆)-alkynyl, (C₂-C₆)-alkenyl, (C₁-C₆)-haloalkyl, (C₁-C₆)-cyanoalkyl, heteroaryl-(C₁-C₆)-alkyl, aryl-(C₁-C₆)-alkyl, (C₁-C₄)-alkylcarbonyl, (C₁-C₄)-alkoxycarbonyl;
or Z¹ and Z² together form an N-(bis(C₁-C₅)-alkyl)sulfanylidene, N-(aryl-(C₁-C₅)-alkyl)sulfanylidene, N-(bis(C₃-C₆)-cycloalkyl)sulfanylidene, N-((C₁-C₅)-alkyl-(C₃-C₆)-cycloalkyl)sulfanylidene group or an N,N-di-(C₁-C₄)-alkylformylidene group.

4. The method as claimed in claim 1, where, in formula (I),
R₁, R₂, R₃ are each independently H, F, Cl, Br, I, CH₃, CF₃, OCH₃, OCF₃;
W is oxygen, sulfur;
n is 0, 1, 2, 3, 4;
Y is H, methyl, ethyl, n-propyl, n-butyl, isobutyl, isopropyl, n-pentyl, n-hexyl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl;
V represents fluoroalkyls having 1 to 4 carbon atoms and 1 to 9, preferably 1 to 6, identical or different halogen atoms with at least one fluorine atom, i.e. partly fluorinated alkyl, perfluoroalkyl, partly fluorinated haloalkyl, where any other halogen atoms present are selected from the group of fluorine, chlorine and bromine, preferably trifluoromethyl, pentafluoroethyl, heptafluoropropyl, nonafluorobutyl, chlorodifluoromethyl, bromodifluoromethyl, dichlorofluoromethyl, bromofluoromethyl, 1-fluoroethyl, 2-fluoroethyl, fluoromethyl, difluoromethyl, 2,2-difluoroethyl, 2,2,2-trifluoroethyl, 2,2-dichloro-2-fluoroethyl, 2-chloro-2,2-difluoroethyl, difluoro-tert-butyl, 1-fluorocyclopropyl, 2-fluorocyclopropyl, 2-fluoro-2-chlorocyclopropyl, 2-bromo-1,1,2-trifluoroethyl, 1,1,2,2-tetrafluoroethyl, 1,2,2,2-tetrafluoroethyl, 2-chloro-1,1,2-trifluoroethyl, 2-chloro-1,1,2,2-tetrafluoroethyl, 1,2,2,3,3,3-hexafluoropropyl, 1-methyl-2,2,2-trifluoroethyl, 1-chloro-2,2,2-trifluoroethyl, 1,2,2,3,3,4,4,4-octafluorobutyl, 1-fluoro-1-methylethyl, n-propoxydifluoromethyl, methoxydifluoromethyl, ethoxydifluoromethyl, n-butoxydifluoromethyl, methoxyethoxydifluoromethyl, n-pentoxydifluoromethyl, 2-methylbutoxydifluoromethyl, 4-methylpentoxydifluoromethyl, n-hexyloxydifluoromethyl, isohexyloxydifluoromethyl, allyloxypropoxydifluoromethyl, methoxypropoxydifluoromethyl, cyclopropylmethoxydifluoromethyl, cyclobutylmethoxydifluoromethyl, but-3-yn-1-yloxydifluoromethyl, pent-4-yn-1-yloxydifluoromethyl, hex-3-yn-1-yloxydifluoromethyl, but-3-en-1-yloxydifluoromethyl, 2,2,2-trifluoroethoxydifluoromethyl, 3,3,3-trifluoropropoxydifluoromethyl, 4,4,4-trifluorobutoxydifluoromethyl, 4-dimethylaminobutoxydifluoromethyl, 2-(1-methylpyrrolidin-2-yl)ethoxydifluoromethyl;
Z¹ is H, chlorine, methyl, ethyl, n-propyl, n-butyl, isobutyl, isopropyl, n-pentyl, n-hexyl, cyclopropyl, cyclobutyl, cyclopentyl, cyclohexyl, prop-1-yn-3-yl, but-2-yn-3-yl, cyanomethyl, prop-1-en-3-yl, but-1-en-4-yl, methylsulfonyl, ethylsulfonyl, cyclopropylsulfonyl, isopropylsulfonyl, n-propylsulfonyl, phenylsulfonyl, p-chlorophenylsulfonyl, m-chlorophenylsulfonyl, m,p-dichlorophenylsulfonyl, p-iodophenylsulfonyl, p-trifluoromethoxyphenylsulfonyl, p-methylphenylsulfonyl; methoxycarbonylmethyl, 1-methoxycarbonylethyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
and
Z² is H, methyl, ethyl, n-propyl, n-butyl, n-pentyl, n-hexyl, cyclopropyl, cyclobutyl, prop-1-yn-3-yl, but-2-yn-3-yl, cyanomethyl, prop-1-en-3-yl, but-1-en-4-yl, methylcarbonyl, ethylcarbonyl, n-propylcarbonyl, tert-butylcarbonyl, cyclopropylcarbonyl, cyclobutylcarbonyl, cyclopentylcarbonyl, cyclohexylcarbonyl, methoxycarbonyl, ethoxycarbonyl, 2-pyridinylmethyl, 2-pyrimidinylmethyl, 2,2-difluoroethyl, 2,2,2-trifluoroethyl, 2,2,3,3,3-pentafluoropropyl, 3,3,2,2-tetrafluoropropyl, 4,4,4-trifluorobutyl
or Z¹ and Z² together are N-(di-n-butylsulfanylidene), N-(di-isopropylsulfanylidene), N-(di-n-propylsulfanylidene), N-(di-n-pentylsulfanylidene), N-(diisobutylsulfanylidene), N-(cyclobutylisopropylsulfanylidene), N-(n-propylisopropylsulfanylidene), N-(cyclopropylisopropylsulfanylidene), N-(isobutylisopropylsulfanylidene), N,N-dimethylformylidene.

5. A treatment for plants, comprising the application of a nontoxic amount, effective for enhancement of the resistance of plants to abiotic stress factors, of one or more of the compounds of the formula (I), or salts thereof, as claimed in claim 1.

6. The treatment as claimed in claim 5, wherein the abiotic stress conditions correspond to one or more conditions selected from the group of drought, cold and hot conditions, osmotic stress, waterlogging, elevated soil salinity, elevated exposure to minerals, ozone conditions, strong light conditions, limited availability of nitrogen nutrients, limited availability of phosphorus nutrients.

7. The method of claim 1 wherein said applying comprises spray application to plants and plant parts in combinations with one or more active ingredients selected from the group consisting of insecticide, attractants, acaricides, fungicides, nematicides, herbicides, growth regulators, safeners, substances which influence plant maturity and bactericides.

8. The method of claim 1 wherein said applying comprises spray application to plants and plant parts in combinations with fertilizers.

9. The method of claim 1 wherein said applying comprises application to genetically modified varieties, the seed thereof, or to cultivation areas on which these varieties grow.

10. The method of claim 1 comprising applying a spray solution which comprises said one or more of the fluoroalkyl-substituted 2-amidobenzimidazoles or salts for enhancing the resistance of plants to abiotic stress factors.

11. A method for increasing stress tolerance in plants selected from the group of useful plants, ornamental plants, lawn types and trees, which comprises the application of a sufficient, nontoxic amount of one or more compounds of the fluoroalkyl-substituted 2-amidobenzimidazoles or salts as claimed in claim 1 to the area where the corresponding effect is desired, comprising application to the plants, the seed thereof or to the area on which the plants grow.

12. The method as claimed in claim 11, wherein the resistance of the plants thus treated to abiotic stress has been increased by at least 3% compared to untreated plants under otherwise identical physiological conditions.

13. A fluoroalkyl-substituted 2-amidobenzimidazole of the formula (I), or salt thereof, 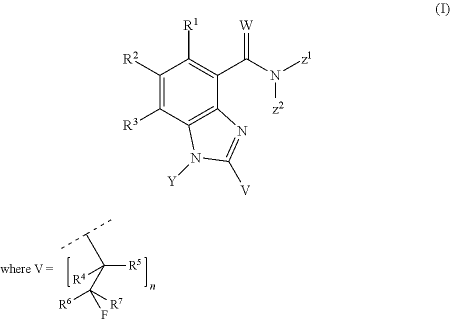
where
R¹, R², R³ are each independently H, halogen, branched or unbranched alkyl, cycloalkyl, alkenyl, alkynyl, aryl, arylalkyl, heteroaryl, alkoxyalkyl, alkylthio, fluoroalkylthio, haloalkyl, alkoxy, haloalkoxy;
R⁴ is H, unbranched alkyl, halogen, haloalkyl, branched alkyl, unbranched alkenyl, branched alkenyl, cycloalkyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁵ is H, unbranched alkyl, halogen, haloalkyl, branched alkyl, cycloalkyl, cycloalkenyl, alkoxy, haloalkoxy;
R⁶ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
R⁷ is H, unbranched alkyl, halogen, haloalkyl, branched alkyl, cycloalkyl, cycloalkenyl, alkoxy, haloalkoxy, cycloalkylalkoxy, alkynylalkoxy, alkenylalkoxy, alkenyloxyalkoxy, alkyloxyalkoxy, alkylaminoalkoxy, bisalkylaminoalkoxy, cycloalkylaminoalkoxy;
n is 0, 1, 2, 3, 4, 5, 6;
W is oxygen, sulfur;
Y is H, unbranched or branched alkyl, cycloalkyl, cycloalkylalkyl, cycloalkenyl, cyanoalkyl, unbranched or branched alkenylalkyl, branched or unbranched haloalkyl, alkynylalkyl, arylalkyl, heteroarylalkyl, alkylcarbonyl, cycloalkylcarbonyl, arylcarbonyl, arylalkylcarbonyl, alkoxycarbonyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, alkoxyalkyl, cyanoalkyl;
Z¹ is H, unbranched or branched alkyl, cycloalkyl, halogen, unbranched or branched alkylalkenyl, branched or unbranched haloalkyl, alkynyl, alkenyl, cyanoalkyl, arylalkyl, heteroarylalkyl, alkylcarbonyl, alkoxycarbonyl, alkylsulfonyl, arylsulfonyl, cycloalkylsulfonyl, alkylsulfinyl, arylsulfinyl, cycloalkylsulfinyl, alkoxycarbonylalkyl;
and
Z² is H, unbranched or branched alkyl, cycloalkyl, branched or unbranched haloalkyl, alkynyl, alkenyl, cyanoalkyl, arylalkyl, heteroarylalkyl, unbranched or branched alkylalkenyl, alkylcarbonyl, alkoxycarbonyl;
or Z¹ and Z² together are part of an optionally substituted sulfilimine or amidine group or form an iminophosphorane,
excluding the compound in which
R¹, R² and R³ are each H; Z¹ and Z² are each H; W is O; Y is H; and V is CF₃.

14. A spray solution for treatment of plants, comprising an amount, effective for enhancement of the resistance of plants to abiotic stress factors, of one or more of the fluoroalkyl-substituted 2-amidobenzimidazoles or salts as claimed in claim 13.
